(19) United States
(12) Patent Application Publication
(10) Pub. No.: US 2026/0229307 A1
(43) Pub. Date: Aug. 6, 2026

(54) CLEAVED NEOEPITOPES

(71) Applicant: IOGENETICS, LLC, Sun Prairie, WI (US)

(72) Inventors: Jane Homan, Sun Prairie, WI (US); Robert D. Bremel, Sun Prairie, WI (US)

(21) Appl. No.: 19/154,063

(22) PCT Filed: Feb. 8, 2024
(86) PCT No.: PCT/US2024/014986

§ 371 Date: Aug. 6, 2025

Related U.S. Application Data
(60) Provisional application No. 63/444,135, filed on Feb. 8, 2023.
(60) Provisional application No. 63/452,766, filed on Mar. 17, 2023.
(60) Provisional application No. 63/468,663, filed on May 24, 2023.

(51) Int. Cl. G16B 20/20; C12Q 1/6827; C12Q 1/6886; G16H 20/10; G16H 50/20

(52) U.S. Cl. CPC G16B 20/20; C12Q 1/6827; C12Q 1/6886; G16H 20/10; G16H 50/20; C12Q 2600/106; C12Q 2600/156

(57) ABSTRACT

The present invention relates to methods for treating, by administration of a cathepsin inhibitor, a subject who is affected by a tumor comprising a specific tumor mutation in a peptide that is cleaved with high frequency by a cathepsin and in which such cleavage prevents the presentation of a neoantigen thereby enabling immune evasion and tumor progression.

### CROSS-REFERENCE TO RELATED APPLICATIONS

This application claims the benefit of U.S. Prov. Appl. 63/444,135 filed Feb. 8, 2023, U.S. Prov. Appl. 63/452,766, filed Mar. 17, 2023, and U.S. Prov. Appl. 63/468,663, filed May 24, 2023, each of which is incorporated by reference herein in their entirety.

### REFERENCE TO A SEQUENCE LISTING

The text of the computer readable sequence listing filed herewith, titled “IOGEN_42082_601_SequenceListing.xml”, created Feb. 7, 2024, having a file size of 293,768 bytes, is hereby incorporated by reference in its entirety.

### FIELD OF THE INVENTION

The present invention relates to methods for treating, by administration of a cathepsin inhibitor, a subject who is affected by a tumor comprising a specific tumor mutation in a peptide that is cleaved with high frequency by a cathepsin and in which such cleavage prevents the presentation of a neoantigen thereby enabling immune evasion and tumor progression.

### BACKGROUND OF THE INVENTION

Each year approximately 2 million cases of cancer are diagnosed in the United States, and approximately 18 million cases worldwide. Cancer accounts for about 20% of all deaths in the United States and approximately 16% worldwide. Ras gene mutations are present in 25% of all cancers and in a high percentage of lung, pancreatic cancer and colorectal cancer, which are three of the four most common types of cancer. KRAS mutations are present in over 90% of pancreatic ductal adenocarcinomas (1). Many interventions for KRAS, HRAS and NRAS mutated cancers have been evaluated, but treatment of the tumors driven by mutations of this gene and its Ras family members has proven challenging (2, 3, 4). Effective interventions for KRAS, HRAS and NRAS and other Ras gene mutated tumors are urgently needed. The present invention, by recognition of the key step in immune evasion of these genes, provides a method to treat such tumors.

### SUMMARY OF THE INVENTION

In the present invention we demonstrate that the detection of a mutation in a tumor driver gene product in which the neoepitope has a high probability of cathepsin cleavage is a biomarker indicating that treatment of the subject with a cathepsin inhibitor would be beneficial to enabling an immune response and halting the tumor progression. In particular, the present invention demonstrates that the presence of a KRAS, NRAS, HRAS mutation, or similar Ras gene mutation, is a biomarker indicating that treatment of the subject with a cathepsin inhibitor would be beneficial.

The invention thus provides a method for treating, by administration of a cathepsin inhibitor, a subject who is affected by a tumor comprising a specific tumor mutation in a peptide that is cleaved with high frequency by a cathepsin and in which such cleavage prevents the presentation of a neoantigen, thereby enabling immune evasion and tumor progression. Administration of the cathepsin inhibitor serves to allow presentation of the neoantigen to T cells and thus prevent or inhibit immune evasion and tumor progression.

Accordingly, in some embodiments the invention addresses the urgent need presented by the high case load of tumors which are driven by mutations of KRAS, HRAS and NRAS. We herein demonstrate that these mutations are especially associated with a high level of cathepsin cleavage which brings about destruction of peptides and prevents presentation of the neoantigen and allows immune evasion. It is contemplated that as mutations in KRAS and related Ras gene products occur predominantly in three sequence hotspots and such mutations are detected in various routine screening assays, the detection of a KRAS, NRAS or HRAS mutation is an indication for administration of a cathepsin inhibitor drug. In some embodiments the mutation in a Ras gene product is detected by sequencing proteins in a biopsy and comparing with sequences in a normal tissue or reference sequence. In other embodiments, the Ras gene product mutation is detected by a biomarker assay and in other instances by an oncogene panel. Most often the Ras gene that is mutated is KRAS, NRAS or HRAS, but other related Ras gene product sequences are closely aligned and subject to the same mutations and associated cathepsin cleavage patterns. KRAS, NRAS and HRAS are identical in the first 86 amino acids of their sequences. Approximately 98% of KRAS, NRAS and HRAS mutations occur at one of three positions, G12, G13 or Q61, and therefore the invention provides for detection of mutations at those positions as an indicator for treatment with a cathepsin inhibitor drug. In one embodiment the mutated peptides that comprise the various mutations at these positions are provided herein, as a more specific indicator of the assay results which would be indicative of the benefit of a cathepsin inhibitor intervention.

While the Ras gene products are exceptional in their susceptibility to cathepsin cleavage at key mutation sites, another embodiment of the invention provides for detection of high probability cathepsin cleavage sites in other mutated proteins which may impact their presentation as neoantigens and facilitate immune evasion. Thus, in some embodiments, a score for the cathepsin cleavage probability, by cathepsin B, L, or S, within any 9mer that comprises the mutant amino acid is provided as an indicator the desirability of administration of a cathepsin inhibitor. In some embodiments, the score is determined as 80% or greater probability of cleavage by cathepsin B, L or S at four or more potential cleavage sites in the 9mer encompassing the mutant amino acid. In other embodiments, the score is set at a more stringent 90% probability at 4 or more potential cleavage sites. As cleavage at any one of the potential cleavage sites in the 9mer can destroy the peptide and prevent presentation to a TCR the score is alternatively set at 80% probability of cleavage at a single site or 90% probability of cleavage at a single site. In further embodiments, as cathepsin B is particularly critical to neoantigen presentation within a tumor, the same scores and indicators for administration of a cathepsin inhibitor drug are applied to just cathepsin B.

In some embodiments, the invention considers more than one cathepsin (e.g., preferably cathepsin B, L and/or S) and therefore addresses cathepsin inhibitors which may act on any of these cathespins. In some preferred embodiments, the cathepsin inhibitor of choice is one which preferentially inhibits cathepsin B. In some embodiments, the cathepsin inhibitor is selected from the group consisting of nitrile derivatives, ketone derivatives, acryl hydrazine derivatives, vinyl sulfonate derivatives, epoxy succinic acids, surugamides, loxistatin derivatives, sulfonamide derivatives and betalactams. In some preferred embodiments, the cathepsin inhibitor is aloxistatin (E64d) or a derivative or analogue thereof. In yet other preferred embodiments, the cathepsin inhibitor of choice is a naturally occurring medicinal product. In other embodiments, cystatin is the cathepsin inhibitor of choice, as a protein or a polypeptide derived therefrom or a cystatin derived molecule may be encoded in a nucleic acid. In further embodiments, peptide cathepsin inhibitor molecules may be the molecule of choice, administered as a peptide or a nucleic acid sequence encoding the same.

In some embodiments, the cathepsin inhibitor may be administered parenterally, by injection or orally, and may be formulated with a suitable pharmaceutical carrier. In some embodiments, administration may be intratumoral. In yet other embodiments administration may be topical, applied to the skin or a mucosal surface.

Mutant proteins with a high frequency of cathepsin cleavage may occur in any tumor, in either driver genes or passengers. In some preferred embodiments described herein, the invention applies to solid tumors. The highest frequency of Ras mutations, and particularly of KRAS, NRAS and/or HRAS mutations, occurs in pancreatic, colorectal and lung cancer. Thus, detection of these cathepsin cleaved mutants and administration of cathepsin inhibitors in these types of cancer is a particularly preferred embodiment. In yet other embodiments, the invention is applied to hematologic cancers. In one particular embodiment, the detection of a NRAS, KRAS or HRAS in acute myeloid leukemia, or any other leukemia, serves as an indication for administration of a cathepsin inhibitor drug of choice.

In further embodiments described herein, it is an objective to ensure the provision of T cell clones which can respond to a neoantigen “rescued” from cleavage by provision of a cathepsin inhibitor by also providing a neoantigen vaccine targeting the uncleaved peptide containing the mutant amino acid. This may be accomplished by a peptide with natural binding affinity for the affected subject's HLA alleles, or by modifying the flanking regions of the peptide, while maintaining the T cell exposed motif intact, to provide improved HLA binding. Embodiments of both approaches are provided herein together with exemplar peptide sequences. While a neoantigen vaccine may be administered as a peptide, in an alternative embodiment the peptide sequence may be encoded in a nucleic acid for administration.

In further embodiments, the administration of a cathepsin inhibitor which will expose otherwise cleaved neoantigens can be followed by administration of a further immunomodulatory intervention to boost the T cell response to the newly exposed neoantigens. In some embodiments, the immunomodulatory intervention is a checkpoint inhibitor; in other embodiments, it is a cytokine or interleukin. In another embodiment, additional protease inhibitors of interest may be added to the regimen.

Overall, the goal of the embodiments described herein is to detect the presence of tumor mutations, and in particular tumor drivers and especially KRAS, NRAS and HRAS mutants, which escape immune surveillance through cleavage and to intervene with a cathepsin inhibitor drug to prevent or reduce the destruction of the neoepitope peptides thus enabling elimination of tumor cells by the T cell response, which may be further enhanced by additional interventions. In some embodiments, a patient is stratified on the basis of the cathepsin cleavage into a patient population that would benefit from administration of a cathepsin inhibitor drug or into a patient population that would not benefit from administration of a cathepsin inhibitor drug.

Accordingly, in some embodiments, the present invention provides methods of treating a subject affected by a tumor comprising: performing or having performed an assay to identify tumor mutations on a nucleic acid sample from the subject to determine if a mutation of a protein encoded by a Ras gene is present; and, if the subject has a mutation of a protein encoded by a Ras gene, treating the subject with a cathepsin inhibitor drug.

In some preferred embodiments, the step of performing or having performed the assay on a nucleic acid sample from the subject to determine if a mutation of a protein encoded by a Ras gene is present further comprises: determining the sequences of genes encoding Ras proteins in the nucleic acid sample; identifying amino acid mutations in the Ras proteins as compared to corresponding wild-type sequences of the Ras protein in the subject or a reference human subject; and, identifying a mutation of a Ras protein.

In some preferred embodiments, the nucleic acid sample is a nucleic acid sample from a tumor biopsy, a nucleic acid sample from a tissue, a cell, collection of cells, blood, plasma, serum, organ secretion, seminal fluid, vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, and a cell-free DNA sample.

In some preferred embodiments, the assay to identify tumor mutations is selected from the group consisting of a hybridization assay, a nucleic acid amplification assay and a sequencing assay. In some preferred embodiments, the nucleic acid amplification assay comprises sequencing the nucleic acid after amplification. In some preferred embodiments, the assay to identify tumor mutations utilizes-an oncogene panel.

In some preferred embodiments, the protein encoded by a Ras gene is selected from the group consisting of KRAS, NRAS, and HRAS. In some preferred embodiments, the amino acid mutation occurs at positions G12, G13 or Q61 of the Ras protein. In some preferred embodiments, the mutations at G12, G13 or Q61 result in a mutated peptide selected from the group consisting of SEQ ID NOs: 56-154. In some preferred embodiments, the mutations at G12, G13 or Q61 result in a mutated peptide selected from the group consisting of SEQ ID NOs: 210-308.

In some preferred embodiments, the mutation of a protein encoded by a Ras gene is in proximity to a predicted cathepsin cleavage site. In some preferred embodiments, the mutation of a protein encoded by a Ras gene is within 9 amino acids of a predicted cathepsin cleavage site with an >80% probability of cleavage. In some preferred embodiments, the cleavage site is on the N terminal side of the mutant amino acid.

In other preferred embodiments, the present invention provides methods of treating a subject having cancer comprising: performing or having performed an assay to identify tumor mutations in a nucleic acid sample from the subject to identify amino acid mutations in tumor proteins in comparison to corresponding wild-type sequences of the protein in the subject or in a reference human subject; identifying 9mer amino acid peptides which comprise the identified amino acid mutations in the tumor proteins; determining the probability of cleavage by a cathepsin of each octomer centered on a potential scissile bond within any 9mer peptide that comprises an identified amino acid mutation in the tumor proteins; identifying the mutated tumor proteins which have a probability of cathepsin cleavage within such octomers that exceeds a predetermined score; and, if the subject has one or more mutated tumor proteins for which the cathepsin cleavage score for peptides comprising the mutant exceeds the predetermined score, treating the subject with a cathepsin inhibitor drug.

In some preferred embodiments, the predetermined score is a greater than 80% probability of cleavage by one or more of cathepsin B, L or S at four or more of the 8 possible cleavage sites in a 9mer that comprises a mutant amino acid. In some preferred embodiments, the predetermined score is a greater than 90% probability of cleavage of one or more of cathepsin B, L or S at four or more of the 8 possible cleavage sites in a 9mer that comprises a mutant amino acid. In some preferred embodiments, the predetermined score is a greater than 80% probability of cleavage by cathepsin B at four or more of the 8 possible cleavage sites in a 9mer that comprises a mutant amino acid. In some preferred embodiments, the predetermined score is a greater than 90% probability of cleavage by cathepsin B at four or more of the 8 possible cleavage sites in a 9mer that comprises a mutant amino acid. In some preferred embodiments, the predetermined score is a greater than 80% probability of cleavage by one or more of cathepsin B, L or S at one or more of the 8 possible cleavage sites in a 9mer that comprises a mutant amino acid. In some preferred embodiments, the predetermined score is a greater than 90% probability of cleavage of one or more of cathepsin B, L or S at one or more of the 8 possible cleavage sites in a 9mer that comprises a mutant amino acid. In some preferred embodiments, the predetermined score is a greater than 80% probability of cleavage by cathepsin B at one or more of the 8 possible cleavage sites in a 9mer that comprises a mutant amino acid. In some preferred embodiments, the predetermined score is a greater than 90% probability of cleavage by cathepsin B at one or more of the 8 possible cleavage sites in a 9mer that comprises a mutant amino acid.

In some preferred embodiments, the step of performing or having performed the assay to identify tumor mutations on a nucleic acid sample from the subject-comprises: determining the sequences of genes encoding tumor proteins in the nucleic acid sample; identifying amino acid mutations in the tumor proteins in comparison to corresponding wild-type sequences of the Ras protein in the subject or a reference human subject; and, identifying a mutation in the tumor protein.

In some preferred embodiments, the nucleic acid sample is a nucleic acid sample from a tumor biopsy, a nucleic acid sample from a tissue, a cell, collection of cells, blood, plasma, serum, organ secretion, seminal fluid, vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, and cell-free DNA sample.

In some preferred embodiments, the assay is selected from the group consisting of a hybridization assay, a nucleic acid amplification assay and a sequencing assay. In some preferred embodiments, the nucleic acid amplification assay comprises sequencing the nucleic acid after amplification. In some preferred embodiments, the assay to identify tumor mutations utilizes-an oncogene panel.

With respect to all of the foregoing embodiments, in some preferred embodiments the cathepsin inhibitor drug inhibits the action of cathepsin L, cathepsin S or cathepsin B. In some preferred embodiments, the cathepsin inhibitor drug preferentially inhibits the action of cathepsin B. In some preferred embodiments, the cathepsin cleavage inhibitor drug is selected from the group consisting of a nitrile derivative, a ketone derivative, an acryl hydrazine derivative, a vinyl sulfonate derivative, an epoxy succinic acid, surugamide, an aloxistatin derivative, a sulfonamide derivative and betalactam cathepsin cleavage inhibitors. In some preferred embodiments, the cathepsin cleavage inhibitor is selected from the group consisting of compounds 1 to 59 and derivatives and salts thereof. In some preferred embodiments, the cathepsin cleavage inhibitor is selected from the group consisting of compounds 1 to 59. In some preferred embodiments, the cathepsin cleavage inhibitor drug is aloxistatin or a derivative thereof. In some preferred embodiments, the cathepsin inhibitor is a naturally occurring medicinal product. In some preferred embodiments, the cathepsin inhibitor is a peptide. In some preferred embodiments, the peptide cathepsin inhibitor is a recombinant peptide. In some preferred embodiments, the peptide is administered encoded in a nucleic acid. In some preferred embodiments, the cathepsin inhibitor is a cystatin protein or polypeptide derived therefrom. In some preferred embodiments, the cystatin protein or polypeptide derived therefrom is administered encoded in a nucleic acid. In some preferred embodiments, the cystatin protein or polypeptide is selected from the group consisting of proteins or polypeptides having SEQ ID NOs: 309-311. In some embodiments the cathepsin inhibitor is administered operably linked to a second molecule, as a genetic fusion or chemical conjugate. In some particular embodiments the second molecule is an antibody or portion thereof, in others it comprises a T cell receptor.

In some preferred embodiments, the cathepsin inhibitor is administered to the subject parenterally. In some preferred embodiments, the cathepsin inhibitor is administered to the subject intratumorally, orally, topically, or to a mucosal surface.

In some preferred embodiments, the tumor is a solid tumor. In some preferred embodiments, the tumor is selected from the group consisting of a pancreatic tumor, a colorectal tumor or a lung tumor. In some preferred embodiments, the tumor is a hematologic cancer. In some preferred embodiments, the hematologic cancer is an acute myeloid leukemia.

In some preferred embodiments, the treatment further comprises the administration of a neoantigen vaccine to the subject. In some preferred embodiments, a mutation of KRAS, NRAS or HRAS is detected at position G12, G13 or Q61 and the treatment further comprises the administration of a neoantigen vaccine that comprises or encodes any of the pentamer T cell exposed motifs in SEQ ID NOs: 1-55. In some preferred embodiments, a mutation of KRAS, NRAS or HRAS is detected at position G12, G13 or Q61 and the treatment further comprises the administration of a neoantigen vaccine that comprises or encodes any of the pentamer T cell exposed motifs in SEQ ID NOs: 155-209. In some preferred embodiments, the neoantigen vaccine is a peptide (or nucleic acid encoding the peptide) that comprises the amino acids of one of said T cell exposed motifs of SEQ ID NOs: 1-55 or 155-209 and in which one or more of the amino acids not within the T cell exposed motif are substituted from those present in the tumor to change the predicted MHC binding affinity. In some preferred embodiments, the neoantigen vaccine comprises a peptides or proteins. In some preferred embodiments, the neoantigen vaccine peptide is encoded in a nucleotide sequence. In some preferred embodiments, the neoantigen vaccine is an RNA vaccine. In yet further embodiments the vaccine is delivered as a viral or virus like particle.

In some preferred embodiments, the methods described above further comprise administering an additional immunomodulatory intervention to the subject. In some preferred embodiments, the immunomodulatory intervention is selected from the group consisting of a checkpoint inhibitor, a cytokine and an interleukin. In some preferred embodiments, the checkpoint inhibitor is selected from the group consisting of Nivolumab, Pembrolizumab, Dostarlimab and Cemiplimab, Atezolizumab, Avelumab, Durvalumab, Ipilimumab, Tremelimumab, and Retalimab. In some preferred embodiments, the immunomodulatory intervention is a protease inhibitor other than a cathepsin inhibitor drug.

### DESCRIPTION OF THE FIGURES

FIG. 1: Cathepsin cleavage profile of KRAS wild type and G12D mutant

X axis shows the positions in the sequence from amino acids 1-189. Black dotted lines indicate positions G12, G13 and Q61. Y axis shows the predicted probability of cathepsin cleavage at each sequential dimer position along the protein where 1=100% probability. Top tier shows cathepsin B, middle tier shows cathepsin S, bottom tier shows cathepsin L.

FIG. 2: Cathepsin profile KRAS G12 mutants

Axes are as in FIG. 1. Each graphic shows the predicted cleavage pattern of a different G12 mutant of KRAS, focusing on the region of amino acids 1-35 which encompasses the mutation.

FIG. 3: Cathepsin profile KRAS G13 mutants

Axes are as in FIG. 1. Each graphic shows the predicted cleavage pattern of a different G13 mutant of KRAS, focusing on the region of amino acids 1-35 which encompasses the mutation.

FIG. 4A-B: Cathepsin profile for additional KRAS mutants.

Axes are as in FIG. 1. FIG. 4A shows the predicted cleavage pattern of the region around mutants Q61H, Q61L and K117N, a less common mutant. FIG. 4B shows the predicted cleavage pattern of the region around three mutants which are represented by only a single case in the Genome Data Commons: A146T, R151T and D33E as representatives of uncommon KRAS mutants.

FIG. 5: Detail of cleavage positions in two KRAS mutants

For G12C and G12D this shows the T cell exposed motifs and the predicted cleavage probability at each position as shown in the column marked (pos) peptide. This is provided as an example; similar data is available for other peptides of interest.

FIG. 6: Comparative cathepsin profiles of additional Ras proteins Axes are as in FIG. 1. Dotted line marks alignment with G13 in KRAS

FIG. 7: Comparison cathepsin profile for TP53 R175H and R248W

Examples of cathepsin cleavage probability profiles for two most common TP53 mutants. Axes as in FIG. 1. Dotted line marks mutant position

FIG. 8: Comparison of detailed cleavage positions in two most common TP53 mutants

FIG. 9: The principal tumor driver proteins have different cathepsin cleavage probability profiles.

The mutations which comprise the top 100 case numbers in Genome Data Commons are shown in the Y axis. The X axis shows the number of dimers in each 9mer comprising the mutant amino acid which exceed 80% probability of cleavage by cathepsin. Hence the maximum score is 8. Cathepsin B is on the left side, cathepsin S is in center and cathepsin L is on the right side. The size of the marker represents the number of cases with this mutant in the GDC database

FIG. 10: Expanded Ras section of FIG. 9

This figure shows all the entries in the RAS section marked with the bracket in FIG. 9.

FIG. 11: Profile of predicted cathepsin cleavage in passenger and driver gene mutations expressed in one tumor biopsy.

Each grouping shows the predicted cathepsin cleavage probability within the T cell exposed motifs for each expressed mutated protein in the tumor biopsy of one subject affected by metastasized colorectal cancer. Y axis shows the predicted probability of cathepsin cleavage at each sequential dimer position along the protein where 1=100% probability. Top tier shows cathepsin B, middle tier shows cathepsin S, bottom tier shows cathepsin L.

FIG. 12: Schematic diagram of potential cleavage site octomers

The potential CSOs are overlayed on potential 9mer peptides containing a mutation (represented as X). There are 8 octomers (spanning 8 potential cleavage dimers) for 9 mutant positions for a total of 72 octomers, but due to overlap, 16 are considered for each 9mer.

FIG. 13: Linear B cell epitopes in Cathepsins B, L and S

Overview of MHC binding, B cell epitopes and topology. The X axis indicates the index position of sequential peptides with single amino acid displacement. The Y axis indicates predicted binding affinity of each peptide in standard deviation units for the protein. The red line shows the permuted average predicted MHC-IA and B (62 alleles) binding affinity of sequential 9-mer peptides with single amino acid displacement. The blue line shows the permuted average predicted MHC-II DRB allele (24 most common human alleles) binding affinity of sequential 15-mer peptides. Orange lines show the predicted probability of B-cell receptor binding for an amino acid centered in each sequential 9-mer peptide. Low numbers for MHC data represent high binding affinity, whereas low numbers equate to high B cell receptor contact probability. Ribbons (red: MHC-I, blue: MHC-II) indicate the 10% highest predicted MHC affinity binding. Orange ribbons indicate the top 25% predicted probability B-cell binding. Horizontal dotted lines demarcate the top 5% of binding affinity for the protein (red MHC I, blue MHC II).

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (see world wide web at ebi.ac.uk/interpro). Human proteome is also understood to include those proteins and antigens thereof which may be over-expressed in certain pathologies, or expressed in a different isoforms in certain pathologies. Hence, as used herein, tumor associated antigens are considered part of the human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The the term peptidase would also include the proteasome which is a complex organelle containing different subunits each having a different type of characteristic scissile bond cleavage specificity. Similarly the term peptidase inhibitor may be used interchangeably with protease inhibitor or inhibitor of any of the other alternate terms for peptidase.

As used herein, the term “exopeptidase” refers to a peptidase that requires a free N-terminal amino group, C-terminal carboxyl group or both, and hydrolyses a bond not more than three residues from the terminus. The exopeptidases are further divided into aminopeptidases, carboxypeptidases, dipeptidyl-peptidases, peptidyl-dipeptidases, tripeptidyl-peptidases and dipeptidases.

As used herein, the term “endopeptidase” refers to a peptidase that hydrolyses internal, alpha-peptide bonds in a polypeptide chain, tending to act away from the N-terminus or C-terminus. Examples of endopeptidases are chymotrypsin, pepsin, papain and cathepsins. A very few endopeptidases act a fixed distance from one terminus of the substrate, an example being mitochondrial intermediate peptidase. Some endopeptidases act only on substrates smaller than proteins, and these are termed oligopeptidases. An example of an oligopeptidase is thimet oligopeptidase. Endopeptidases initiate the digestion of food proteins, generating new N- and C-termini that are substrates for the exopeptidases that complete the process. Endopeptidases also process proteins by limited proteolysis. Examples are the removal of signal peptides from secreted proteins (e.g. signal peptidase I,) and the maturation of precursor proteins (e.g. enteropeptidase, furin, etc.). In the nomenclature of the Nomenclature Committee of the International Union of Biochemistry and Molecular Biology (NC-IUBMB) endopeptidases are allocated to sub-subclasses EC 3.4.21, EC 3.4.22, EC 3.4.23, EC 3.4.24 and EC 3.4.25 for serine-, cysteine-, aspartic-, metallo- and threonine-type endopeptidases, respectively. Endopeptidases of particular interest are the cathepsins, and especially cathepsin B, L and S known to be active in antigen presenting cells. Cathepsin B may function as an endo peptidase or an exopeptidase. Many different cathepsin are known, most of them located in lysosomes. They can be classified as serine proteases, aspartyl proteases and cysteine proteases. Cathepsins B, L and S are cysteine proteases.

As used herein, the term “immunogen” refers to a molecule which stimulates a response from the adaptive immune system, which may include responses drawn from the group comprising an antibody response, a cytotoxic T cell response, a T helper response, and a T cell memory response. An immunogen may stimulate an upregulation of the immune response with a resultant inflammatory response or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein, the term “T-cell epitope” refers to a polypeptide sequence which when bound to a major histocompatibility protein molecule provides a configuration recognized by a T-cell receptor. Typically, T-cell epitopes are presented bound to an MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally. As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, an “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. When referring to the HLA alleles on both chromosomes in a subject we refer to “HLA genotype”.

Each class of MHC-Is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (February 2010) lists 948 class I and 633 class II molecules, many of which are represented at high frequency (>1%). MHC alleles may differ by as many as 30-aa substitutions. Different polymorphic MHC alleles, of both class I and class II, have different peptide specificities: each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary.

The digits before the first colon describe the type, which often corresponds to the serological antigen carried by an allele, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or ‘Q’. The suffix ‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein, the term “affinity” refers to a measure of the strength of binding between two members of a binding pair, for example, an antibody and an epitope, and an epitope and a MHC-I or II allele. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=−RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50 or inhibitory concentration 50, that concentration at which 50% of the peptide is displaced. Likewise ln(ic50) refers to the natural log of the ic50.

The term “K_{off}”, as used herein, is intended to refer to the off rate constant, for example, for dissociation of an antibody from the antibody/antigen complex, or for dissociation of an epitope from an MHC molecule.

Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. This is also commonly referred to as the Z-scale. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an MHC allele means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′) 2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc.

“Adjuvant” as used herein encompasses various adjuvants that are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

As used herein “immunoglobulin” means the distinct antibody molecule secreted by a clonal line of B cells; hence when the term “100 immunoglobulins” is used it conveys the distinct products of 100 different B-cell clones and their lineages.

As used herein, the term “principal component analysis”, or as abbreviated “PCA”, refers to a mathematical process which reduces the dimensionality of a set of data (Wold, S., Sjorstrom, M., and Eriksson, L., Chemometrics and Intelligent Laboratory Systems 2001. 58:109-130.; Multivariate and Megavariate Data Analysis Basic Principles and Applications (Parts I&II) by L. Eriksson, E. Johansson, N. Kettaneh-Wold, and J. Trygg, 2006 2^{nd} Edit. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements. The application of PCA generates numerical coefficients (descriptors). The coefficients are effectively proxy variables whose numerical values are seen to be related to underlying physical properties of the molecules. A description of the application of PCA to generate descriptors of amino acids and by combination thereof peptides is provided in PCT US2011/029192 incorporated herein by reference in its entirety. Unlike neural nets PCA do not have any predictive capability. PCA is deductive not inductive.

As used herein, the term “vector” when used in relation to a computer algorithm or the present invention, refers to the mathematical properties of the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. “Viral vector” as used herein includes but is not limited to adenoviral vectors, adeno-associated viral vectors, lentiviral vectors, retroviral vectors, poliovirus vectors, measles virus vectors, flavivirus vectors, poxvirus vectors, and other viral vectors which may be used to deliver a peptide or nucleic acid sequence to a host cell.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.).

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

As used herein, the term “motif” refers to a characteristic sequence of amino acids forming a distinctive pattern.

The term “Groove Exposed Motif” (GEM) as used herein refers to a subset of amino acids within a peptide that binds to an MHC molecule; the GEM comprises those amino acids which are turned inward towards the groove formed by the MHC molecule and which play a significant role in determining the binding affinity. In the case of human MHC-I the GEM amino acids are typically (1,2,3,9). In the case of MHC-II molecules two formats of GEM are most common comprising amino acids (−3,2, −1,1,4,6,9,+1,+2,+3) and (−3,2,1,2,4,6,9,+1,+2,+3) based on a 15-mer peptide with a central core of 9 amino acids numbered 1-9 and positions outside the core numbered as negative (N terminal) or positive (C terminal).

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Illustrative examples of immunopathologies include, but are not limited to, B-cell lymphoma, T-cell lymphomas, Systemic Lupus Erythematosus (SLE), allergies, hypersensitivities, immunodeficiency syndromes, radiation exposure or chronic fatigue syndrome.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

“Somatic hypermutation” (SHM), as used herein refers to the process by which variability in the immunoglobulin variable region is generated during the proliferation of individual B-cells responding to an immune stimulus. SHM occurs in the complementarity determining regions.

“T-cell exposed motif” (abbreviated “TCEM”), as used herein, refers to the subset of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide typically comprise positions 4, 5, 6, 7, 8 of a 9-mer. The amino acids which comprise the TCEM in an MHC-II binding peptide typically comprise 2, 3, 5, 7, 8 or −1, 3, 5, 7, 8 based on a 15-mer peptide with a central core of 9 amino acids numbered 1-9 and positions outside the core numbered as negative (N terminal) or positive (C terminal). As indicated under pMHC, the peptide bound to a MHC may be of other lengths and thus the numbering system here is considered a non-exclusive example of the instances of 9-mer and 15mer peptides.

“Pentamer amino acid motif” or “pentameric amino acid motif” as used herein refers to a set of five amino acids arranged in the same configuration as a T cell exposed motif, but not necessarily bound in a MHC. Thus a pentamer amino acid motif may refer to a contiguous sequence of five amino acids in the format XXXXX, or to a discontinuous pentamer in the format XX~X~XX or X~X~~X~XX, where X is any amino acid. A T cell exposed motif is defined by its protrusion from an MHC and exposure to the T cell receptor when the underlying peptide is bound by a MHC molecule. A pentamer amino acid motif is the same pattern of amino acids occurring in a protein in the absence of any MHC binding. A pentamer amino acid motif only becomes a T cell exposed motif if the peptide in which it lies is appropriately cleaved out of a protein and the host's MHC alleles have the necessary affinity for binding that peptide to expose the pentamer motif.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in the MHC. The T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs.

“Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“uTOPE™ analysis” as used herein refers to the computer assisted processes for predicting binding of peptides to MHC and predicting cathepsin cleavage, described in PCT US2011/029192, PCT US2012/055038, and US2014/01452, each of which is incorporated herein by reference in its entirety.

“Isoform” as used herein refers to different forms of a protein which differ in a small number of amino acids. The isoform may be a full length protein (i.e., by reference to a reference wild-type protein or isoform) or a modified form of a partial protein, i.e., be shorter in length than a reference wild-type protein or isoform. In accordance with the convention adopted by the Genome Data Commons, the isoform selected as the reference for numbering amino acid positions is the longest identified in Uniprot https://www.uniprot.org.

“Immunostimulation” as used herein refers to the signaling that leads to activation of an immune response, whether the immune response is characterized by a recruitment of cells or the release of cytokines which lead to suppression of the immune response. Thus, immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances such elimination may include apoptosis of the responding T cells.

“Frequency” as used herein in reference to the human proteome and microbial databases including the gastrointestinal microbiome reference database refers to the count of occurrences or count of a particular amino acid motif in that database or proteome.

“hPPF” as used herein refers to the human proteome pentamer frequency or the count of occurrences of a particular amino acid pentameric motif in the human proteome.” hPPF” I refers to the count of pentamers which are in the configuration presented by a TCEM I i.e. a contiguous pentamer like positions 4,5,6,7,8 within a 9mer. “hPPF II” refers to the count of pentamers which are in the configuration presented by a TCEM II i.e. a discontinuous pentamer like positions 2,3,5,7,8 in a central core 9mer of a 15mer. “giPPF I” and “giPPF II” refer to the corresponding pentameric amino acid motif counts within a representative gastrointestinal microbiome protein database.

A “rare TCEM” as used herein is one which is completely missing in the human proteome or present in up to only five instances in the human proteome. Similarly a TCEM may be rare with respect to the gastrointestinal microbiome reference database or other database if it is missing or only occurs five or less times.

“Adverse immune response” as used herein may refer to (a) the induction of immunosuppression when the appropriate response is an active immune response to eliminate a pathogen or tumor or (b) the induction of an upregulated active immune response to a self-antigen or (c) an excessive up-regulation unbalanced by any suppression, as may occur for instance in an allergic response.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting. “Clonal population” or “clonal line” may be used as a synonym for clonotype.

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein.

“Cytokine” as used herein refers to a protein which is active in cell signaling and may include, among other examples, chemokines, interferons, interleukins, lymphokines, granulocyte colony-stimulating factor tumor necrosis factor and programmed death proteins.

“MHC subunit chain” as used herein refers to the alpha and beta subunits of MHC molecules. A MHC II molecule is made up of an alpha chain which is constant among each of the DR, DP, and DQ variants and a beta chain which varies by allele. The MHC I molecule is made up of a constant beta macroglobulin and a variable MHC A, B or C chain.

As used herein the term “repertoire” is used to describe a collection of molecules or cells making up a functional unit or whole. Thus, as one non limiting example, the entirely of the B cells or T cells in a subject comprise its repertoire of B cells or T cells. The entirety of all immunoglobulins expressed by the B cells are its immunoglobulinome or the repertoire of immunoglobulins. A collection of proteins or cell clonotypes which make up a tissue sample, an individual subject or a microorganism may be referred to as a repertoire.

As used herein “mutated amino acid” refers to the appearance of an amino acid in a protein that is the result of a nucleotide change, a missense mutation, or an insertion or deletion or fusion.

A “tumor mutation” as used herein is a mutation occurring in a cancer cell or tumor cell. A tumor mutation may comprise a nucleotide mutation, for instance a C>T, or T>A. In a protein a tumor mutation comprises a mutated amino acid as defined above.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

“T cell receptor” as used herein refers to the heterodimer (two proteins) located on the surface of a t cell that engage with the epitope peptide bound by an MHC molecule (pMHC). T cell receptor is abbreviated herein as TCR.

“TRAV” as used herein refers to the T cell receptor alpha variable region family or allele subgroups and “TRBV” refers to T cell receptor beta variable region family or allele subgroups as described in IMGT (see world wide web at imgt.org/IMGTrepertoire/Proteins/index.php #C; imgt.org/IMGTrepertoire/Proteins/taballeles/human/TRA/TRAV/Hu_TRAVall.html.TRAV comprises at least 41 subgroups, with some having sub-subgroups. TRBV comprises at least 30 subgroups. Most combinations of alpha and beta variable region subgroups are encountered. “hTRAV” refers to human TRAV. As used here in a “receptor bearing cell” is any cell which carries a ligand binding recognition motif on its surface. In some particular instances a receptor bearing cell is a B cell and its surface receptor comprises an immunoglobulin variable region, the immunoglobulin variable region comprising both heavy and light chains which make up the receptor. In other particular instances a receptor bearing cell may be a T cell which bears a receptor made up of both alpha and beta chains or both delta and gamma chains. Other examples of a receptor bearing cell include cells which carry other ligands such as, in one particular non limiting example, a programmed death protein of which there are multiple isoforms.

As used herein the term “bin” refers to a quantitative grouping and a “logarithmic bin” is used to describe a grouping according to the logarithm of the quantity.

As used herein “immunotherapy intervention” is used to describe any deliberate modification of the immune system including but not limited to through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, cytokine or recombinant cytokine or cytokine enhancer, including but not limited to a IL-15 agonist, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, an enhancer of NK (natural killer) cells, an interleukin including but not limited to variants of IL15, superagonists, and a nutritional or dietary supplement. Immunomodulatory interventions also includes protease inhibitors, including but not limited to inhibitors of cathepsins, and may include but are not limited to molecules from the group comprising nitrile derivatives, ketone derivatives, acryl hydrazine derivatives, vinyl sulfonate derivatives, epoxy succinic acids, surugamides, loxistatin derivatives, sulfonamide derivatives and betalactams, natural medicinal derivatives such as caffeic acid and chlorogenic acid. Additional cathepsin inhibitors are members of the cystatin family, including but not limited to the stefins and cystatin C. The immunomodulatory intervention may also include radiation or chemotherapy to ablate a target group of cells. The impact on the immune response may be to stimulate or to down regulate.

“Checkpoint inhibitor” or “checkpoint blockade” as used herein refers to a type of drug that blocks certain proteins made by some types of immune system cells, such as T cells, and some cancer cells. These proteins help keep immune responses in check, limit the duration of T cell responses, and can prevent T cells from killing cancer cells. When these proteins are blocked, the “brakes” on the immune system are released and T cells are able to kill cancer cells better. Examples of checkpoint proteins found on T cells or cancer cells include, but are not limited to, PD-1/PD-L1 and CTLA-4/B7-1/B7-2 and LAG-3. Multiple check point inhibitors have been developed or are in development and include, but are not limited to, PD-1 inhibitors (e.g., Nivolumab, Pembrolizumab, Dostarlimab and Cemiplimab), PD-L1 inhibitors (e.g. Atezolizumab, Avelumab, Durvalumab), CTLA-4 inhibitors (e.g.Ipilimumab, Tremelimumab), and LAG-3 inhibitors (e.g. Retalimab).

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed at the world wide web at uniprot.org/docs/cdlist.

As used herein “microbiome” refers to the constellation of commensal microorganisms found within the human or other host body, inhabiting sites such as the gastrointestine, skin the urogenital tract, the oral cavity, the upper respiratory tract. While most frequently referring to bacteria, the microbiome also may include the viruses in these sites, referred to as the “virome”, or commensal fungi.

As used herein “tumor associated antigens” are antigens in proteins commonly upregulated in a tumor, or different types of tumor, but which are not mutated nor specific to that tumor and not differentiated form a wild type protein.

“Pattern” as used herein means a characteristic or consistent distribution of data points.

As used herein “presentome” refers to the multiplicity of peptides bound in MHC and simultaneously presented on the surface of antigen presenting cells. Mass spectroscopy detects some but not all peptides which are part of the presentome.

“Neoepitope” as used herein refers to a novel epitope amino acid motif or antigen created as the result of introduction of a mutation into an amino acid sequence. Thus, a neoepitope differentiates a wildtype protein from its mutant-bearing tumor protein homolog when such mutant is presented to T cells or B cells. A “neoantigen” is a neoepitope which elicits an immune response.

“Tumor specific antigen” or “tumor specific epitope” is used herein to designate an epitope or antigen that differentiates a mutated tumor protein from its unmutated wildtype homologue. Thus, a neoantigen or neoepitope is one type of tumor specific antigen.

As used herein “driver” mutations are those which arise early in tumorigenesis and are causally associated with the early steps of cell dysregulation. Driver mutations occur in oncogenes and tumor suppressor genes. Driver mutations are usually shared by all clonal offspring arising from the initial tumor cells and offer some additional fitness benefit to the clonal line within its microenvironment.

In contrast “passenger” is applied herein to mutations of genes and their products in a tumor which are not in oncogenes or tumor suppressor genes and which offer no particular benefit of fitness to the cell. Passengers may serve as biomarkers on tumor cells and may enable some immune evasion. Passenger mutations may differ at different time points in its development and among different parts of a tumor or among metastases. Any tumor may comprise any number of driver and passenger mutations. “Driver and passenger” are terms largely interchangeable with “trunk and branch” mutations.

“Oncogene” as used herein to describe a gene and gene product “oncoprotein” which have the capability to cause dysregulation of cell growth. Such dysregulation most often occurs when an oncogene is mutated.

“Tumor suppressor gene” as used herein refers to a gene and gene product that normally controls cell replication or nucleic acid replication or apoptosis. When mutated and such functions fail a mutated tumor suppressor may become a driver of tumor progression.

“Personal mutations” as used herein refers to mutations found in the tumor of a particular subject and not commonly shared with other affected subjects. In contrast “common mutations” are used to describe those mutations which occur in many tumors and many types of cancer. Illustrative examples are TP53 R175H, KRAS G12C, BRAF R640M.

“Bespoke peptides” or “bespoke vaccine” as used herein refers to a peptide or neoantigen or a combination of peptides, or nucleic acid encoding peptides, that are tailored or personalized specifically for an individual patient, taking into account that patient's HLA alleles and mutations.

“Heteroclitic” and “heteroclitic peptide” as used herein refers to a peptide in which amino acid substitutions have been made in the groove exposed motifs to alter the binding affinity to a particular HLA allele while maintaining the TCEM constant.

As used herein “TCGA” refers to The Cancer Genome Atlas on the world wide web at www.cancer.gov/about-nci/organization/ccg/research/structural-genomics/tcga.

As used herein Genome Data Commons or GDC refers the National Cancer Institute database of tumor mutations maintained at the University of Chicago on the world wide web at gdc.cancer.gov/.

As used herein a “polyhydrophobic amino acid” refers to a short chain of natural amino acids which are hydrophobic. Examples include, but are not limited to, leucines, isoleucines or tryptophans where these are assembled in multimers of 5-15 repeats of any one such amino acid. As a non-limiting example, a poly leucine comprising 8 leucines would be an example of a polyhydrophobic amino acid.

A “lipid core peptide system”, as used herein, refers to subunit vaccine comprising a lipoamino acid (LAA) moiety which allows the stimulation of immune activity. A combination of T cell stimulating epitopes or T and B cell stimulating epitopes are linked to a LAA. Multiple different constructs can be created with of different spatial orientation or LAA lengths (e.g. C12 2-amino-D,L-dodecanoic acid or C16, 2-amino-D,L-hexadecanoic acid). When dissolved in a standard phosphate buffer LCP particles form and the particles facilitate uptake by antigen presenting cells.Different LAA chain lengths lead to different particle sizes.

As used herein, the term “cleavage site octomer” refers to the 8 amino acids spanning (four on each side) the bond at which a peptidase cleaves an amino acid sequence. Cleavage site octomer is abbreviated as CSO. “Cathepsin cleavage site octomer” is used herein where the peptidase is a cathepsin.

“Cathepsin” as used herein may refer to any cathepsin encoded by the human genome including but not limited to cathepsins B, C, F, H, K, L, O, S, V, W, and X and whether they act as endopeptidases or carboxyexopeptidases or aminopeptidases.

As used herein, a “BAM” file is a compressed binary version of a Sequence Alignment File “SAM” file wherein the all nucleotides are aligned to a reference genome. A “BAM slice” is a subset of the entire genome defined by genome coordinates. The HLA locus is located on Chromosome 6. In one particular instance a BAM slice is defined to contain just the HLA locus.

“Antigen presenting cell” (APC) as used herein refers to cells which are capable of presentation of peptides to T cells bound to MHC molecules. This includes but is not limited to the so called “professional” antigen presenting cells comprising but not limited to dendritic cells, B cells, and macrophages, and Langerhans cell, but also the so called non-professional antigen presenting cells which carry MHC molecules.

“PBMC” as used herein refers to peripheral blood mononuclear cells.

“Multiplex” as used herein refers to a combination of peptides or nucleotides each of which provides a different epitope. Such combination may be delivered as individual epitopes in a single mixture or as a linked chain of epitopes, or the nucleotides that encode them, separates by appropriate spacer sequences.

As used herein “KRAS” refers to the Kirsten Rat sarcoma viral oncogene homolog, a GTPase exemplified by Uniprot ID P1116. “NRAS” refers to the neuroblastoma RAS viral oncogene exemplified by UniProt ID P01111. “HRAS” refers to the Harvey rat sarcoma viral oncogene homolog exemplified by UniProt ID P01112. The Ras gene family refers to GDP-GTP regulatory proteins with significant homology to the prior three referenced examples. Any of these, including KRAS, HRAS and NRAS, are referred to herein as “Ras genes” and proteins therefrom as “Ras gene products” or “Ras proteins”.

“ERAP” as used herein refers to endoplasmic reticulum aminopeptidases, which are enzymes that trim amino acid residues from the NH2 terminus of polypeptides thus playing role in various biological processes, including trimming peptides for MHC binding.

“Bagging” or bootstrap aggregation as used herein refers to the statistical process used in predictive modeling wherein an ensemble method uses bootstrap replicates of the original training set to fit predictive model.

“Biomarker assay” as used herein refers to the testing for a genetic, proteomic or metabolic indicator linked to a particular disease. In the present context biomarker assay refers to the detection of genomic or proteomic mutations in genes and gene products associated with cancer. A variety of methods are employed in biomarker assays including, but not limited to, PCR (polymerase chain reactions) assays of various types, hybridization assays, capture assays, assays for antibodies and T cell responses. Many biomarker assays are commercially available and FDA approved.

“Oncogene panel” as used herein refers to one form of biomarker assay wherein a tissue or biopsy from a subject who is affected by, or at risk of being affected, a tumor is tested for a selected array of common tumor mutations.

As used herein the term “nucleic acid sample” refers to nucleic acid obtained from an organism from the Monera (bacteria), Protista, Fungi, Plantae, and Animalia Kingdoms. The nucleic acid may also be obtained from a virus. Nucleic acid samples may be obtained from a from a patient or subject, from an environmental sample, or from an organism of interest (e.g., both cellular and circulating cell-free DNA (cfDNA) obtained from from tissue, a cell, collection of cells, blood, plasma, serum, organ secretion, semen (seminal fluid), vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, or any other bodily fluid comprising a desired nucleic acid or cfDNA), DNA obtained from biopsies, and DNA obtained from cells, secretions, or tissues from the lymph gland, breast, liver, bile ducts, pancreas, mouth, stomach, colon, rectum, esophagus, small intestine, appendix, duodenum, polyps, gall bladder, anus, prostate, endometrium, vagina, ovary, cervix, skin, bladder, kidney, lung, and/or peritoneum). In other embodiments, the target nucleic acid may be obtained from a sample that contains diseased tissue or cells, or is suspected of containing diseased tissue or cells (e.g., a sample that is cancerous, or contains cancerous tissue or cells, or is suspected of being cancerous or suspected of containing cancerous tissue or cells). In some embodiments, the nucleic acid sample is obtained from a subject that has a disease or disorder (e.g., cancer), is suspected of having the disease or disorder, or is being screened to determine the presence of the disease or disorder. In some embodiments, the nucleic acid sample is circulating cell-free DNA (cell-free DNA or cfDNA), for instance DNA found in the blood and is not present within a cell. As would be recognized by one of ordinary skill in the art based on the present disclosure, cfDNA can be isolated from a bodily fluid using methods known in the art. Commercial kits are available for isolation of cfDNA including, for example, the Circulating Nucleic Acid Kit (Qiagen). The nucleic acid sample may result from an enrichment step, including, but is not limited to antibody immunoprecipitation, chromatin immunoprecipitation, restriction enzyme digestion-based enrichment, hybridization-based enrichment, or chemical labeling-based enrichment.

### DESCRIPTION OF THE INVENTION

Genomic mutations are an inevitable consequence of cell replication. The vast majority of mutations occurring are inconsequential. Of mutations that occur in the ~ 2% of the genome which encodes proteins, most are not expressed in protein or have no functional effect. The majority of mutations encoded and expressed in proteins are eliminated by immune surveillance. Those which escape immune surveillance, and which are found most commonly in tumor biopsies, are those which have most capacity to drive tumor progression, either as oncogene products or as tumor suppressors (6). A wide range of additional mutated “passenger” gene products may also be identified in tumor biopsies (7). The combination of mutations found in a tumor is unique to each affected subject.

The critical step which allows the immune response to identify and target aberrant tumor cells is the binding of short peptides, comprising the mutant amino acids, to major histocompatibility molecules (MHC) and their presentation to T cells, including both cytotoxic CD8+ T cells and T helper CD4+ cells (8). These peptides are neoepitopes unique to the tumor. However very few neoepitopes give rise to an immune response; few neoepitopes are neoantigens.

Mutations which are detected at the time of clinical diagnosis of tumors are those which have successfully evaded immune surveillance and elimination by one of several means. These include mutations occurring in peptides that fail to bind to an MHC molecule, mutations in peptides with the mutant amino acids positioned within the binding groove of the MHC and thus hidden from T cell recognition, and mutations which are exposed to T cells as a component of a rare amino acid combination which does not attract an adequate and effective array of cognate T cells (9). All tumors are unique in the combination of mutations they carry, the percentages of each mutated protein in the tumor tissue, the expression of the protein in the tumor, and the degree to which the binding and positioning of the mutant amino acid allows recognition of a tumor specific neoantigen by T cells. The combination of the MHC alleles carried by the affected subject, and the breadth and diversity of the subject's prior immune exposures and thus T cell repertoire are thus also factors in determining which neoantigens are exposed to T cells.

A further reason that a tumor mutation may not be recognized by a T cell is if the peptide comprising the mutation is cleaved and thus prevented from binding to an MHC, so that a mutation-specific neoantigen is precluded from being presented to a T cell receptor (TCR). In the absence of presentation of a neoepitope peptide bound to an MHC, no effective mutation-specific T cell response that could eliminate the tumor cell can occur. The present invention addresses neoepitopes which are not recognized as neoantigens because the peptide comprising the mutant amino acid is cleaved by cathepsin, in the tumor, or in an antigen presenting cell, or in both.

### Role of Cathepsins in Epitope Presentation

Cathepsins have been extensively studied and are widely distributed in different tissues. There are many different cathepsins. In humans there are 11 known cysteine cathepsins (cathepsins B, C, F, H, K, L, O, S, V, X and W), of which the majority are endopeptidases (10, 11). The cathepsins have differential expression and roles. Cathepsin S and L have critical functions in antigen presenting cells (12, 13, 14), while others, including cathepsin B, are more ubiquitously expressed (11). Cathepsin B functions both as an endopeptidase and as an exopeptidase.

Enzymatic cleavage, typically by cathepsins, is important to generate short peptides of a length that can bind MHC molecules (12, 15, 16, 17, 18). Short peptides may bind at one end (the C terminal end) and are then further trimmed to fit the MHC groove, typically by endoplasmic reticulum aminopeptidases (ERAP) (19, 20, 21). A different situation arises when, rather than fitting a short peptide into an MHC binding groove, an enzyme cleaves the peptide in close proximity to a mutant amino acid. This precludes a tumor specific epitope from ever being presented to a TCR.

Cathepsins, as a class of proteases, thus play a dual role, essential to both the presentation of antigens in professional and non-professional antigen presenting cells but also as a mechanism for cleavage of peptides which prevents their presentation as antigens. Each cathepsin varies in its contribution to each of these functions. Cathepsin B contributes more to peptide destruction, while cathepsin S and L assist the presentation of antigens in antigen presenting cells. While many studies have focused on the role of cathepsins L and S (12, 15, 22, 23) in antigen presentation, there has been much less attention focused on the role of cathepsins in destroying potential tumor specific neoantigens. In particular there has been no attention to the cleavage by cathepsin of specific tumor neoepitopes as a mode of immune evasion. As immune evasion is a key factor in the role of oncogenes and tumor suppressors, enabling them to continue driving tumor progression, we investigated cathepsin cleavage as a factor in neoantigen presentation.

### Cathepsins in Tumors

Cathepsin B is expressed in many cell types (11) and in particular is upregulated in many tumor cells. The upregulation of cathepsin B in cancer cells has been widely reported, in vitro (24, 25, 26) and in vivo in mouse models (27, 28) and in humans (29). Cathepsin B up-regulation has been identified as an adverse prognostic indicator in human cancers (30, 31). Knockout of cathepsin B in a mouse model has been linked to reduced tumor progression (28, 32, 33). The role of cathepsin B in tumor progression has been variously attributed to proteolysis of the extracellular matrix, angiogenesis, increases metastasis, autophagy, and apoptosis (29, 34, 35, 36). However, the potential role of cathepsin B cleavage of neoepitopes and thus enabling immune evasion has not been examined in relation to specific oncogene proteins or tumor suppressors.

The activity of cathepsins is sensitive to pH and temperature (37, 38, 39, 40, 41) which implies that the activity of cathepsin B may vary between tumors, tumors in different locations, or at different sites within a tumor.

### Established Methods for Cathepsin Cleavage Prediction

The inventors have previously developed predictive algorithms for determining the probability for cathepsin B, L and S cleavage (42, 43) (See e.g., U.S. Pat. No. 11,069,427 incorporated herein by reference in its entirety). Briefly, these algorithms were developed using the following steps. Multiple chemical and physical properties reported amino acids were used to derive sets of principal components to provide proxies for each amino acid that encompass their variables. Drawing on large sets of experimentally determined peptide cleavage events (39, 44) for each cathepsin B, L or S, sets of octomer peptides which were cleaved and sets which were uncleaved were used to train a classifier and generate an ensemble of predictive equations to predict cleavage or non-cleavage. These were further refined by bagging (boot strap aggregation) repetition of multiple random subsets of the experimental dataset. To generate a cathepsin cleavage probability profile of a protein of interest (in the present instance of a mutated tumor protein) each sequential octomer in the protein of interest is subjected to multiple repetitions of the ensemble of predictive equations which in effect vote on whether the octomer is cleaved at its central dimer or not. The output is characterized as a probability score of 0-100% for cleavage of each possible dimer in the 9mer that constitutes a potential neoepitope bound by a MHC I, or the central 9mer of a 15mer bound by an MHC II. The probability of a mutated neoepitope being cleaved may be expressed according to the individual dimer cleavage probability or as the aggregate of cleavage probabilities across the neoepitope peptide.

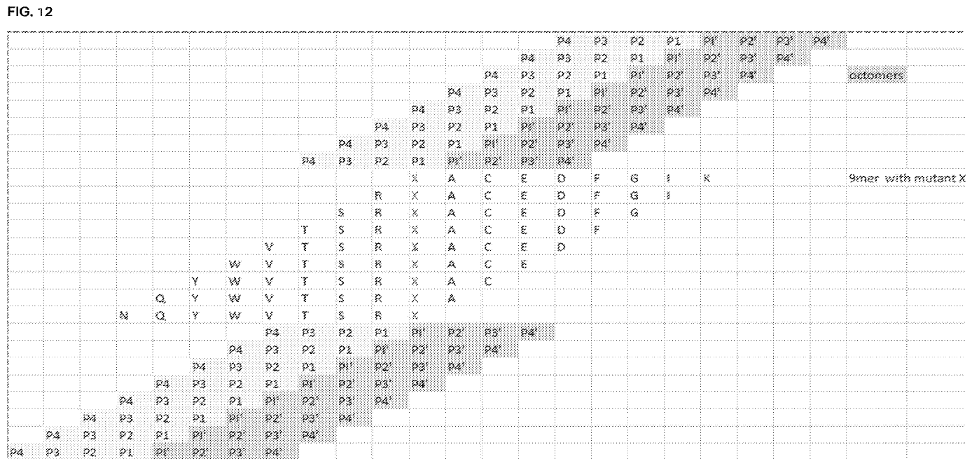

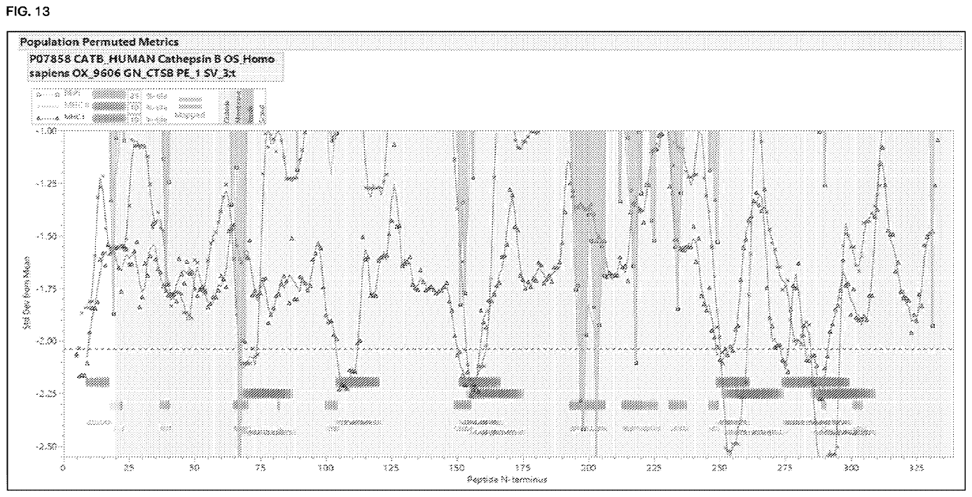

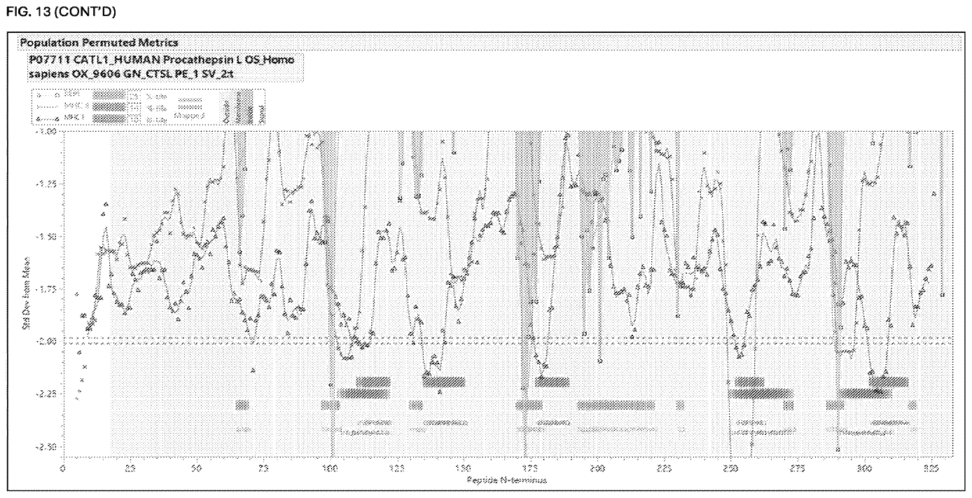

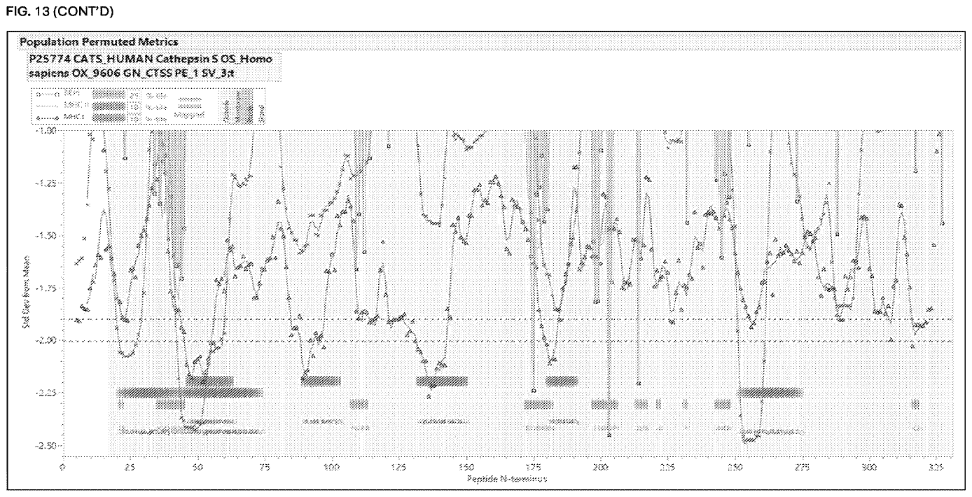

An MHC class I 9mer is comprises eight potential scissile bonds. Cleavage of any these bonds will result in a loss of exposure of the TCEM within that 9mer to a T cell. This is represented schematically in FIG. 12. Thus, a quantitative scoring metric or each TCEM pentamer is a summation of the number of scissile bonds in the peptide that are predicted to be cleaved by the enzyme with a probability of cleavage greater than a threshold. Thus, in preferred embodiments, the probability of cleavage by a cathepsin of each octomer centered on a potential scissile bond (i.e., four amino acids on either side) in any 9mer peptides that comprise the identified amino acid mutations in the tumor protein is determined. FIG. 12 provides a schematic depiction of how the octomers overlap with each of the 9mers that comprise a mutant amino acid (depicted as “X”). In practice a threshold of 0.8 (80%) is used and a maximum score of 8 occurs when all bonds have a high probability of being cleaved.

$$Score = {\sum }_{i = 1}^{8} ( cleavage prob scissle bond ( i ) \geq threshold )$$

By applying these predictive algorithms it is possible to draw a cathepsin profile for any protein showing the probability of cleavage at any amino acid dimer of interest in the protein, by cathepsin L, S or B and to derive a cleavage probability score for each 9mer that may play a role in tumor mutation recognition. When applied to a mutated tumor protein of interest this allows a prediction of whether a peptide comprising a mutant amino acid is likely to be excised as a peptide of suitable size for MHC binding and presentation and exposure of the mutant amino acid to the TCR, while in a further embodiment it predicts whether the peptide comprising the mutant amino acid is destroyed or retained intact to allow presentation.

Application of cathepsin cleavage prediction algorithms to well characterized tumor driver gene products comprising known mutational hotspots shows, as described more fully below, that in a subset of oncogenes and tumor suppressors cathepsin cleavage has a high probability of destruction of the neoepitope. Similarly, some proteins with “passenger” mutations may exhibit a high probability of cleavage that would also prevent T cell recognition of these neoepitopes. The cleavage of a neoepitope thus prevents presentation on an MHC, renders the mutation immunologically invisible and creates a mechanism of tumor immune evasion.

In one embodiment herein, we describe the application of cathepsin prediction algorithms to Ras gene products, which show a very high level of predicted cleavage at, and adjacent to, critical common mutation sites. When applied to some other known drivers, for instance TP53, there is a far lower rate of predicted cathepsin cleavage, indicating that other means of immune evasion dominate. When applied to mutated passenger gene products, either high or low probability cleavage is encountered on an individual basis.

Application of cathepsin cleavage prediction algorithms indicate very high predicted probability of cleavage on the immediate N terminal side of the most common mutation sites in KRAS at G12 and G13 positions, and Q61 (and the corresponding conserved regions of NRAS and HRAS). The implication of this is that cleavage of these peptides by cathepsin B precludes, or markedly reduces, the presentation within the tumor of tumor specific KRAS neoantigens. This would render the mutants immunologically invisible and enable immune evasion and would limit any effective neoantigen vaccination. Cleavage by cathepsins S and L would also reduce the presentation of the neoepitopes arising from these mutations in antigen presenting cells. The Ras gene products are examples of the most extreme case of cathepsin cleavage preventing neoantigen presentation, however other recognized tumor driver gene products are also affected.

Such a mode of immune evasion would be a failure of neoantigen presentation and immune recognition that is mutation sequence-specific. It is independent of, but in addition to, the described effects that cathepsins in tumors, and most especially cathepsin B, have on the integrity of the extracellular matrix, enhancement of metastasis autophagy and apoptosis.

### Neoantigen Based Interventions Directed to KRAS

The Ras genes, and more specifically KRAS, NRAS and HRAS, have presented a particular enigma as there has been increasing effort to develop personal neoantigen vaccines. While KRAS mutant-specific T cells can be stimulated by vaccination with peptides identical to those of the uncleaved KRAS peptide vaccination spanning the mutant sites, and such T cells can be detected by in vitro assays, they have had little or no impact on tumor progression in vivo (45, 46, 47, 48).

There is one report of autologous transfer of KRAS mutant specific T cells to a patient with a resulting beneficial impact on tumor progression (49). In this case the T cell exposed amino acid motif to which the subject responded did not comprise the mutant G12D. The T cells were responsive to a peptide on the C terminal flank of the mutation that had a high affinity for C*08:02, which would have placed the mutant D in a MHC pocket position and was a peptide which may have been released by cathepsin cleavage occurring in a position on the N terminal side of the mutant rather than a peptide cleaved by cathepsin.

Interestingly, despite extensive mass spectroscopy screening of cancer cells and reporting of detected peptides, the only KRAS 9mer peptide reported as detected by this method is located near the C terminal end of the protein (US20210162004A1); no G12 unmutated peptides or other common mutants have apparently been reported. This would be consistent with the absence of intact potential tumor specific KRAS (and related Ras) neoantigens in tumors.

Not surprisingly there are many human proteins which comprise the same T cell exposed pentamer motifs (TCEM) that comprise the mutant amino acids found in the various G12 and G13 KRAS mutants. Many of these motifs are found in other Ras-like proteins and GTPases and have similar predicted patterns of cleavage. However, the same pentameric TCEM are also found in the context of different flanking amino acid sequences in other proteins (e.g., apolipoprotein L6, collagen IA1, others depending which peptide from a KRAS mutant is compared) and in a context of flanking amino acids where they have lower predicted rates of cleavage. The same TCEM are also found in hundreds of proteins within the gastrointestinal microbiome, many of which have different flanking sequences and may have lower probability of cathepsin cleavage. This suggests that an adequate population of precursor cognate T cell clones exists which could respond to the KRAS mutants, if in fact the peptides were intact and presented. Indeed, the prior work on vaccination with KRAS peptides in their uncleaved form indicates that this is so, albeit without impact on the tumor (46).

### Cathepsin Inhibitors

Detection of KRAS mutations, or tumor mutations in other tumor driver or passenger proteins of interest, may be determined in the course of tumor biopsy exome or whole genome sequencing and comparison with normal tissue from the affected subject (71). However, many oncogene panels and biomarker tests are designed to identify KRAS, NRAS or HRAS mutations (72, 73, 74, 75, 76). Almost all KRAS, NRAS and HRAS mutations occur at one of the three hotspots G12, G13 or Q61 (1), each of which has a high level of cathepsin cleavage, as shown below. In these cases the presence of a KRAS mutation can be indicative that a cathepsin inhibitor may enhance the immune recognition of the neoepitope. For other tumor drivers and passengers individual analysis of mutation position, by sequencing and tumor-normal comparison, and cathepsin cleavage profile analysis is needed to determine whether cathepsin cleavage is contributing to immune evasion and thus whether the affected subject may benefit from a cathepsin inhibitor.

In tumors with identified Ras encoded or other highly cleaved neoepitopes, the administration of a cathepsin B inhibitor may be combined with other immunotherapeutic interventions. These may include the administration of a neoantigen specific vaccine, administered as a peptide or nucleic acid encoding a peptide, or in other delivery vehicles including but not limited to viral or viral-like particles. in order to stimulate T cell clones cognate for the neoantigen “rescued” from cathepsin cleavage. Administration of a cathepsin B inhibitor may be combined with a checkpoint inhibitor or other immunotherapeutic drug.

Accordingly, the present invention arises from the recognition that peptides which are cleaved by cathepsins are prevented from being bound by MHC molecules and presented to T cells and thus escape immune surveillance and elimination. Peptides (or their encoding nucleic acids) corresponding to the uncleaved sequence, when used as a neoantigen vaccine, may elicit a cognate T cell response that is detected in assays, but as the peptide is cleaved where it occurs in the tumor the T cells do not encounter a target in the tumor and are thus the tumor is unresponsive to such a vaccine.

Any peptide which comprises a mutated amino acid has the potential to be recognized as a neoantigen that elicits an immune response, but in practice very few neoepitopes are neoantigens. Mutations which are detected in tumor biopsies are those which, for one reason or another, have evaded the host's immune response. Identifying those mutated peptides which comprise a potential neoepitope but which are prevented from becoming neoantigens by cathepsin cleavage offers the opportunity to inhibit the cathepsin cleavage and allow presentation of the neoantigen. This in turn will allow immune recognition and effective immune elimination of tumor cells. Such a response depending on the restoration of a neoepitope can occur in an immunocompetent subject but would not be observed in a mouse model comprising a SCID or other immune-incompetent mouse.

Different cathepsins have different roles in different cells. Cathepsin S and L in antigen presenting cells, including dendritic cells, are essential to the excision of short peptides which are suitable for binding to MHC molecules, in some cases following further trimming by ERAP. Cathepsin B is more widely distributed in other tissues and known to be upregulated in tumors. It is thus desirable to enable the function of cathespin S and L in antigen presenting cells, while inhibiting the cleavage by cathepsin B in those tumors which have mutant peptides (neoepitopes) that are subject to a high rate of destruction by cathepsin B.

Many common tumor drivers (oncogenes and suppressors) are not readily cleaved by cathepsin. Therefore, differentiating those which are is important to selecting cancer affected subjects who may benefit from treatment to reduce the cathepsin cleavage. In one embodiment, therefore, we provide a method for providing a cathepsin cleavage probability profile of each mutated tumor protein to determine if it would be rendered more readily subject to immune recognition by inhibition of cathepsins.

In a further embodiment we demonstrate that the Ras oncogene family, and in particular KRAS, NRAS and HRAS, have an extremely high probability of cleavage of those peptides which comprise the most common tumor mutations. Mutations in these 3 oncogenes are present in over 25% of all cancers. While Ras mutations may be identified in many cancers, in particular KRAS mutants are found in over 90% of pancreatic ductal adenocarcinomas, and in over 50% of colorectal cancers and 30% of lung adenocarcinoma. NRAS mutations are frequently present in melanomas and acute myeloid leukemias.

The sequences of KRAS, NRAS and HRAS are identical in positions 1-86 and highly conserved in the remaining sequence. Approximately 98% of the mutations in these proteins occur at in one of 3 positions: G12, G13 or Q61. Mutations at Q61 are less common in KRAS than in NRAS and HRAS. Cathepsin cleavage probability is extremely high in peptides that comprise any of the mutations occurring at G12 and G13 and also high in peptides comprising Q61 mutations.

While a detailed analysis by comparison of tumor and normal sequences will reveal the presence of mutations of KRAS, NRAS or HRAS, many biomarker assays are available that identify KRAS mutations by PCR assays of tissue or cell free DNA. Detection of KRAS mutations is included in essentially all oncogene panel assays. The detection of a KRAS mutation in a tumor, whether by exome sequencing, whole genome sequencing, oncogene panel, PCR assay of tissue or of cell free DNA would be indicative of the potential benefit of treatment with a cathepsin B inhibitor.

Comparisons of tumor biopsy and normal tissue exome sequences allows identification of the full range of both driver and passenger mutations in a tumor, and characterization of all types of mutation including missense, indels and fusions. Many other characteristics can be identified, including HLA binding and exposure of the mutant amino acid when bound in the MHC. An assessment can be made of the potential precursor frequency of cognate T cells by assessing the frequency of the T cell exposed motif relative to the human proteome and indicators of exogenous stimulation such as the gastrointestinal microbiome. A cathepsin cleavage probability profile can be derived for every relevant mutated protein in the tumor biopsy and consideration given to whether this indicates “escape by cleavage” in some drivers or passengers which may indicate a beneficial effect of administration of a cathepsin inhibitor to the subject.

Causing a previously cleaved peptide neoepitope to be presented as an uncleaved peptide neoantigen will result in an effective cytotoxic response only if T cells cognate for the newly presented T cell exposed motif are present. In the case of the TCEM that comprise the mutated amino acids in the common mutants of KRAS and related RAS, we show that both human proteome and the gastrointestinal microbiome have peptides comprising a large number with TCEM matching those in the mutant positions of KRAS. This indicates that quorum of cognate T cell clone should be present if the mutant peptide is presented to TCR.

Nevertheless, additional interventions can ensure that unmasking the mutant peptide neoantigens by inhibiting cathepsin cleavage is more likely to lead to an effective immune response eliminating tumor cells. This includes co-administration of other immunomodulatory interventions, including but not limited to, checkpoint inhibitors.

In addition, the priming of cognate T cell clones by administration of a neoantigen vaccine comprising the uncleaved peptides can ensure that active clones are available as the neoantigens are presented. Such vaccinal peptides are selected according to a particular subject's HLA alleles and their binding thereto. Selection of the neoantigen peptides may depend on natural HLA binding, when feasible for the affected subject's HLA alleles, or may comprise heteroclitic peptides with amino acid substitutions in the flanking positions that better optimize binding. See, e.g., PCT US2020/037206 and U.S. Prov. Appl. 63/452,766, both of which are incorporated herein by reference in their entirety.

### Cathepsin Inhibitors

A number of cathepsin inhibitors are known to the art and may be utilized in the methods of treatment described herein. These include, but are not limited to, nitrile derivatives, ketone derivatives, acryl hydrazine, vinyl sulfonate derivatives, epoxy succinic acid, betalactams, surugamides, loxistatin derivatives, sulfonamide derivatives and many other products (50, 51, 52, 53, 54). Products with cathepsin inhibitory characteristics have been extensively reviewed and the properties of each discussed (50, 55, 56, 57, 58). Useful cathepsin inhibitors are also described in the following United States patents and patent publications, each of which is incorporated by reference herein in its entirety: U.S. Pat. Nos. 8,748,649, 8,680,152, 8,518,874, 8,450,373, 8,431,733B2, U.S. Pat. Nos. 8,367,732, 8,324,417, 8,211,897, 8,163,735, 8,143,448, U.S. Pat. Nos. 8,106,059, 8,013,186, 8,013,183, 7,893,112, 7,893,093, 7,781,487, 7,737,300, 7,696,250, 7,662,849, 7,608,592, 7,547,701, 7,488,848, US20150191459, US20140256698, US20140221478, US20140018421, US20120329837, US20120282267, US20120190714, US20110281879, US20110172310, US20110046406, US20100305331, US20100266537, US20090312571, US20090270415, US20090234127, US20090233909, US20090203629, US20090170909, US20090023781, US20080293819, US20080214676, US20080161254, US20070287699. In some embodiments, downregulation of cathepsin B is achieved by administration of a siRNA from cathepsin B. In yet other embodiments the siRNA may be linked to or co-administered with a neoepitope vaccine. In still other embodiments, an antibody drug conjugate (ADC) may be utilized in which a cathepsin inhibitor drug is conjugated to an antigen binding molecule (e.g., an antibody or fragment thereof), most preferably an antigen binding molecule that binds to an epitope on a tumor protein of interest. In yet others a cathepsin inhibitor drug is conjugated to a T cell receptor cognate for an intact T cell epitope on the tumor cell or adjacent cells as a means of directing the cathepsin inhibitor to the environs of the tumor cell.

The best characterized inhibitor of cathepsin B is aloxistatin, (also known as E64d or loxistatin. Aloxistatin has been evaluated in a number of diseases over the last approximately 30 years (64, 66). This includes clinical trials in human muscular dystrophy (67), albeit with no clinical benefit. This trial did however demonstrate the safety of aloxistatin in humans and provided insights into its pharmacodynamics. Aloxistatin has been proposed as an intervention for traumatic brain injury and other neurologic disorders including Alzheimer's disease. (60, 68, 69, 70, 71, 72). In the Sars-COV-2 pandemic aloxistatin was also evaluated as a intervention for COVID disease (73) (see also US20220370360A1, US20230053688A1, WO2022265697A1, each of which is incorporated herein by reference in its entirety).

In light of the differential sites of expression of cathepsin B and other cathepsins, the inhibition of cathepsin B is most desired. Natural peptidic cathepsin B inhibitors comprise 3 groups: aldehydes, aziridinyl peptides and epoxysuccinyl peptides. The first two groups include miraziridine and tokaramide A isolated from a marine sponge and leupeptin and YM-51084 peptides isolated from Streptomyces ((74) (63) (75) Among the epoxysuccinyl peptides the best recognized is E64 originally isolated from Aspergillus japonicus. Many derivatives of E64 have been evaluated, the best studied being E64d (aloxistatin) which has improved cell permeability and adsorption. Some further derivatives of E64d show improved selectivity for cathepsin B. Some derivates have shown further selectivity for Cathepsin B including CA074 (E64c) (76). The relative activity of these have been reviewed (63). Aloxistatin has been evaluated in humans for its potential effect in muscular dystrophy, thereby also demonstrating its safety and enabling pharmacokinetic studies (67).

Non-peptidic natural compounds include various flavonoids including amentoflavone, methylamentoflavone and dimethylamentoflavone. Additional groups of irreversible cathepsin B inhibitors include aziridines, 1,2,4, -thiadiazoles, acycloxymethylketones, beta lactams, and organotellurium compounds. Reversible cathepsin B inhibitors include members of the groups of aldehydes, ketones, cyclopropenones and cyclometallated compounds and nitriles (63). As many of the effective anti-cathepsin moieties are peptides this opens the way to provide them as recombinant molecules delivered as peptides or as nucleic acids encoding the peptides, separately or as a component of a neoantigen vaccine. Furthermore the cathepsin inhibitor peptides may be delivered as a fusion, or otherwise in operable association, with a peptidic or protein molecule which facilitates cell uptake. Such molecules may comprise Fc receptors, may be an immunoglobulin or a component thereof, or may comprise a fatty acid moiety. Furthermore, antibodies targeting proteins upregulated and expressed on the tumor cell surface or the extracellular matrix thereof may be conjugated or fused to a cathepsin inhibitor for targeted delivery to the tumor site.

The examples cited here of cathepsin inhibitors are intended to provide an overview of currently available cathepsin inhibitors, and particularly cathepsin B inhibitors; such a summary is not considered limiting and additional cathepsin inhibitors may be added in the future.

Regulation of cathepsin in vivo is effected by proteins of the cystatin family which inhibit cathepsins at pico and nanomolar levels (77). Disruption of cystatin expression has been associated with cancer progression.

Cathepsin inhibitors may be quite specific as to which cathepsin they inhibit (75), for example a cathepsin inhibitor being specifically selected to target only cathepsin C (U.S. Pat. No. 10,238,633B2, incorporated herein by reference in its entirety) and another specific to cathepsin S (See www.opnme.com) while showing selection against cathepsin B. In a preferred embodiment of the present invention an inhibitor effective against cathepsin B is desirable.

The beneficial effect of cathepsin inhibitors in some cancers is acknowledged in the literature, but is attributed to the more general mechanisms noted above such as the effect on the extracellular matrix or apoptosis (61, 62, 77) rather than immune evasion.

In some preferred embodiments, the cathepsin inhibitor is an irreversible covalent inhibitor (e.g., aloxistatin). In some preferred embodiments, the cathepsin inhibitor is a reversible cathepsin inhibitor. In some preferred embodiments, the cathepsin inhibitor is a non-covalent inhibitor.

Suitable cathepsin inhibitors include, but are not limited to, the following compounds described in Siklos et al., Acta Pharmaceutical Sinica B (2015) 5 (6): 506-519. (69)

Epoxysuccinate cysteine protease inhibitors (e.g., compounds 1 to 12 and derivatives and salts thereof; aloxistatin is E-64d).

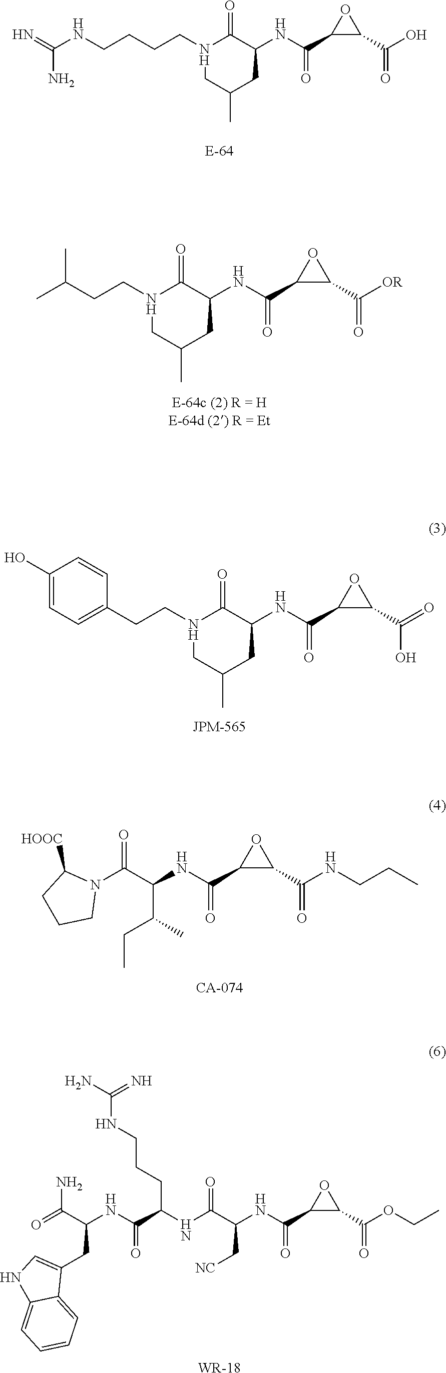

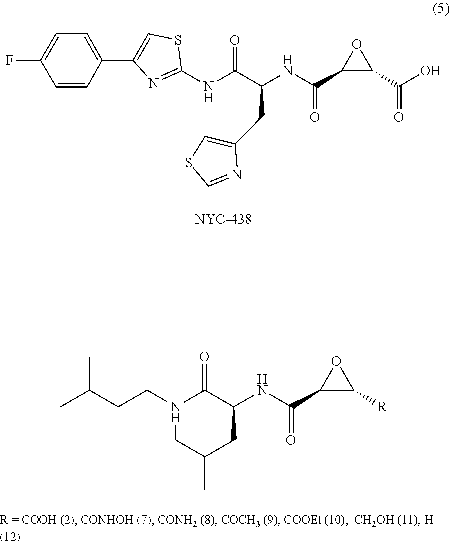

Aziridine and β-lactone cysteine protease inhibitors (e.g., compounds 13-15 and derivatives and salts thereof).

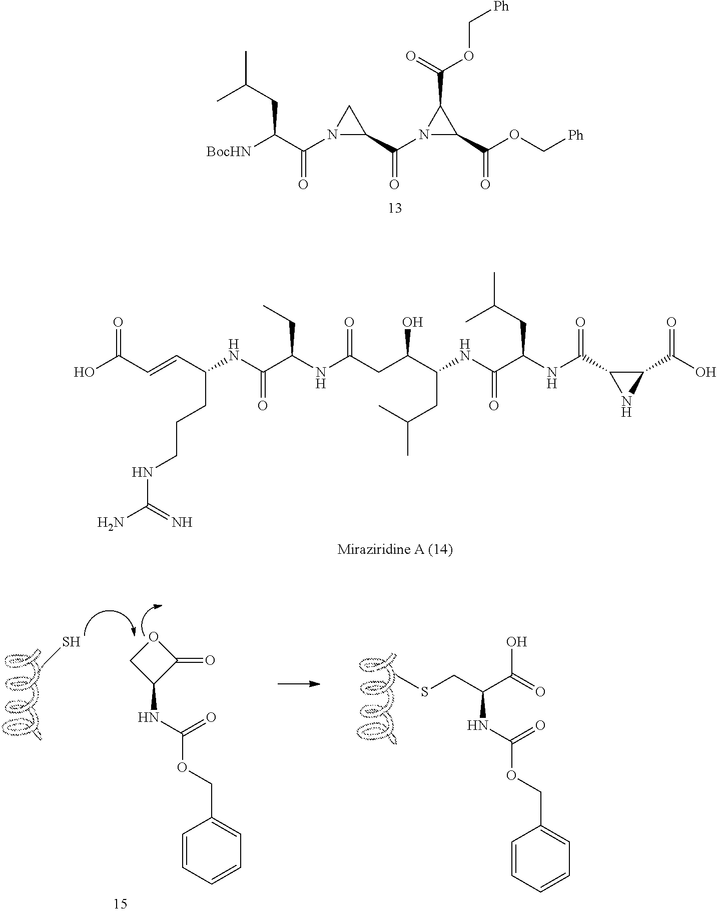

Michael acceptor warheads in cysteine protease inhibitors (e.g., compounds 16 to 20 and derivatives and salts thereof).

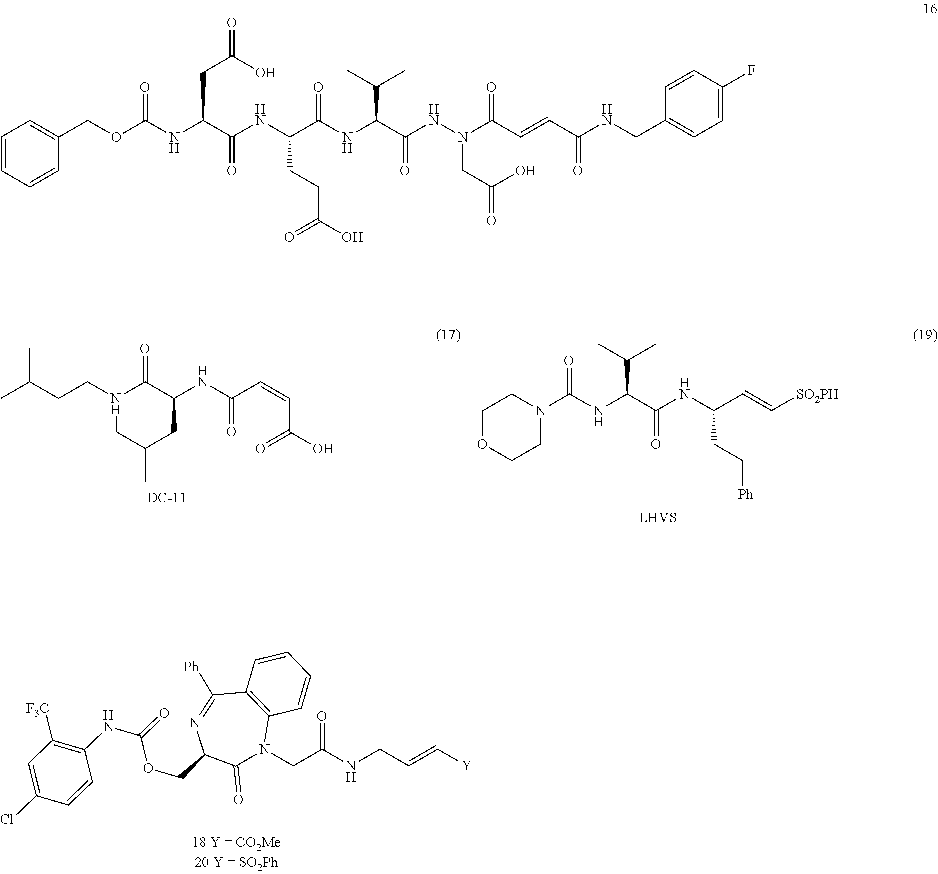

Diazomethyl, acyloxy and other ketone cysteine protease inhibitors (e.g., compounds 21 to 28 and derivatives and salts thereof).

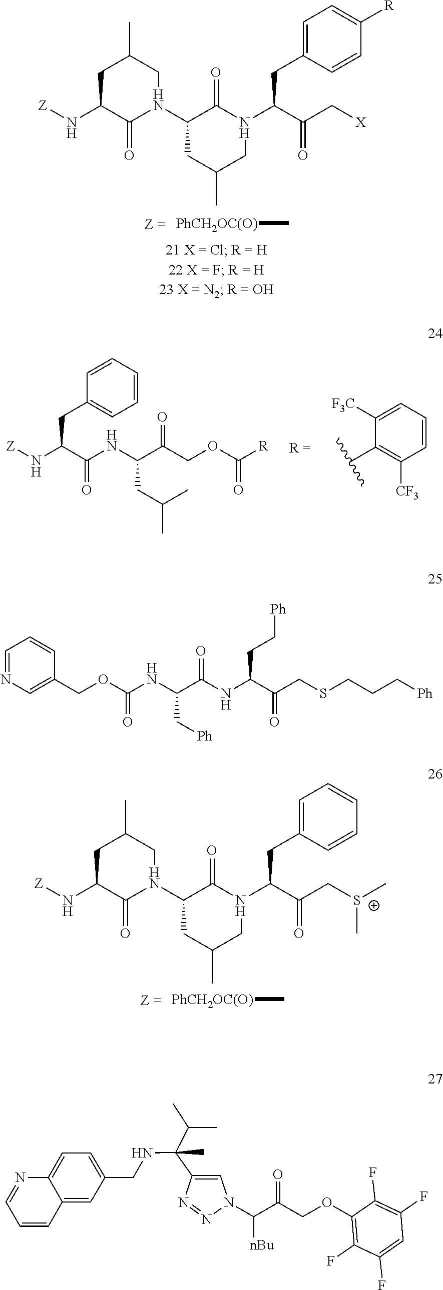

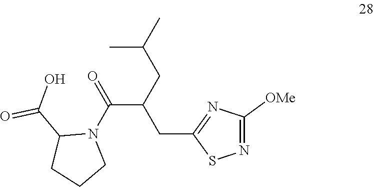 [SMILES: COc1nsc(CC(CC(C)C)C(=O)N2CCCC2C(=O)O)n1]

Aldehyde and cyclopropenone inhibitors (e.g., compounds 29-35 and derivatives and salts thereof)

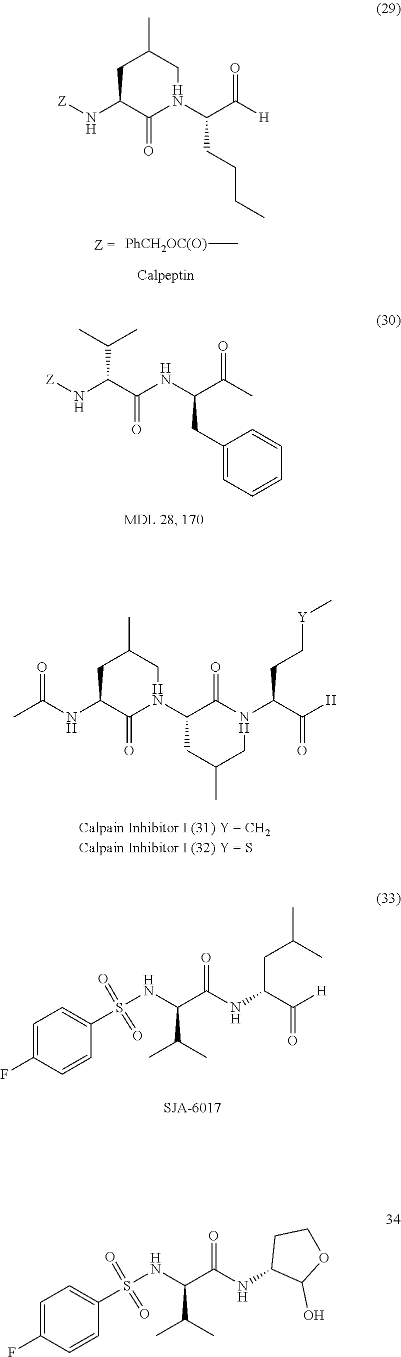

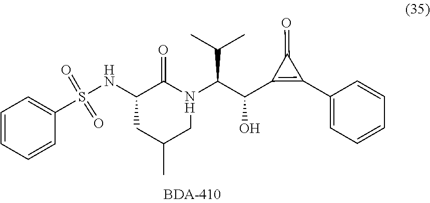

Ketoamide and ketoheterocycle cysteine protease inhibitors (e.g., compounds 36-45 and derivatives and salts thereof.)

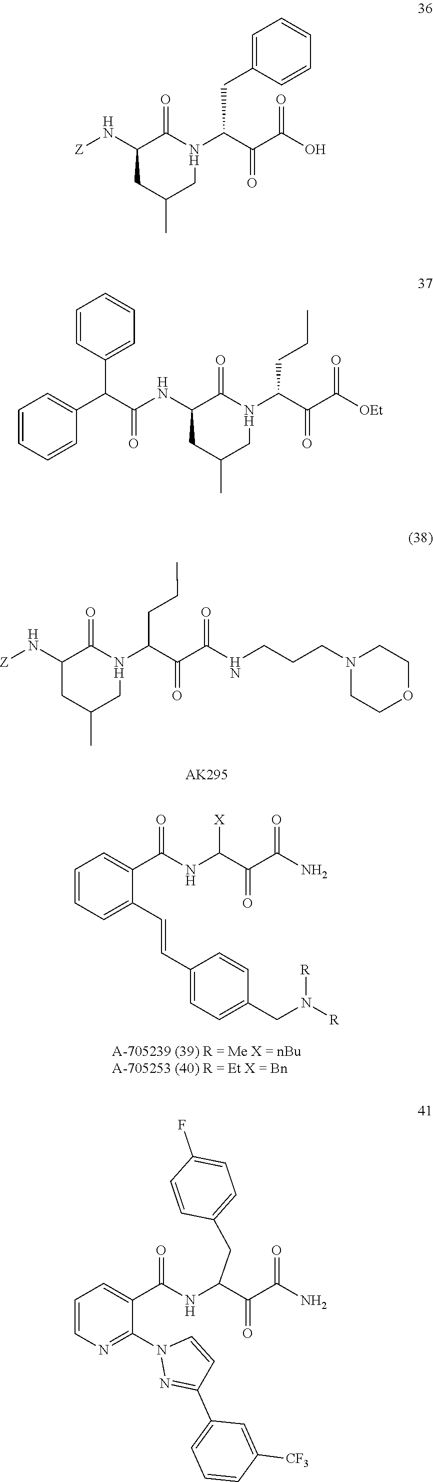

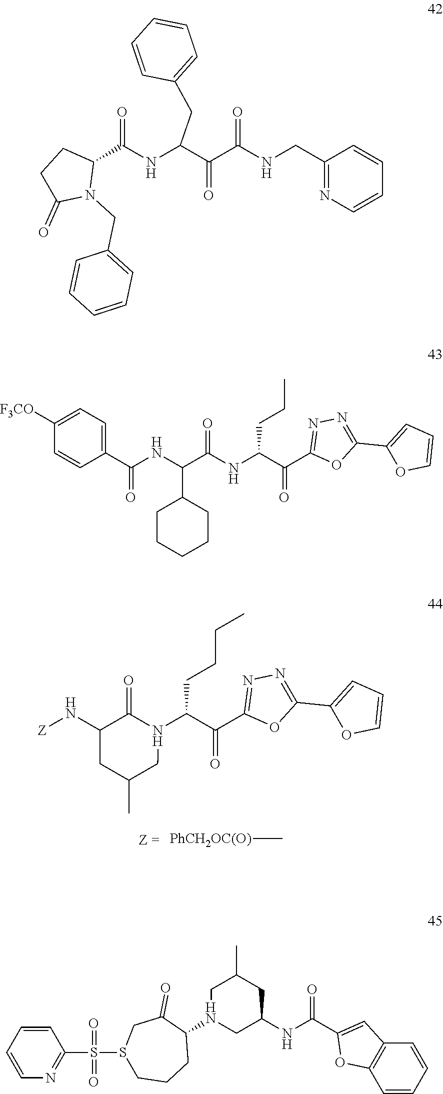

Nitrile and carbodiimide inhibitors (e.g., compounds 46 to 55 and derivatives and salts thereof).

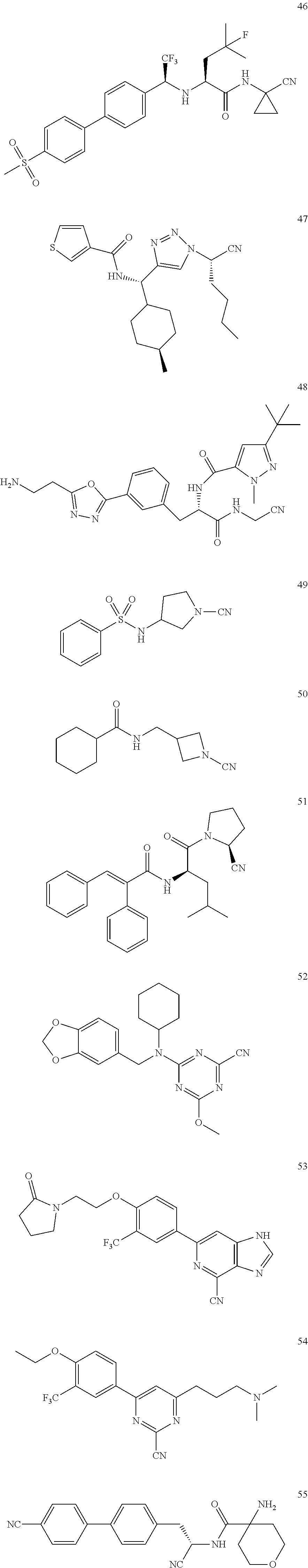

Allosteric inhibitors (e.g., compounds 56 to 59 and derivatives and salts thereof).

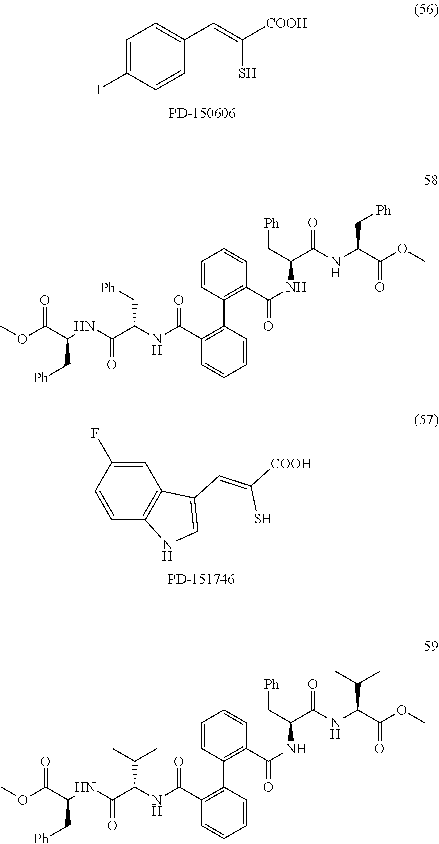

Non-peptidic natural compounds include astaxanthin and various flavonoids including amentoflavone, methylamentoflavone and dimethylamentoflavone. Additional groups of irreversible cathepsin B inhibitors include aziridines, 1,2,4, -thiadiazoles, acycloxymethylketones, beta lactams, and organotellurium compounds. Reversible cathepsin B inhibitors include members of the groups of aldehydes, ketones, cyclopropenones and cyclometallated compounds and nitriles (57).

In some further embodiments, antigen binding proteins directed to cathepsin epitopes are utilized as cathepsin inhibitors. In some embodiments, an antibody to cathepsin is prepared and a recombinant version of the antibody or a molecule comprising the variable regions of that antibody are provided as a means of reducing or neutralizing the activity of the cathepsin. In other embodiments, an antibody targeting an epitope in a protein upregulated in the tumor cells or the extracellular matrix is provided as a fusion or conjugate to a cystatin or a subsequence of cystatin and provided to target the cystatin to the tumor cell. Examples of upregulated tumor proteins to which such targeting antibodies may be directed include, not only those mutated, but also unmutated proteins such as brevican, or MAGEA1 or NY-CSO. In still other embodiments, a portion of the antibody, such an scFv or Fab fragment is utilized. In still further embodiments, the CDRs of the antibodies, heavy and light chain variable regions, or scFv's utilizing the CDRs and/or heavy and light chain variable regions are used in a soluble T cell receptor or fusion thereof.

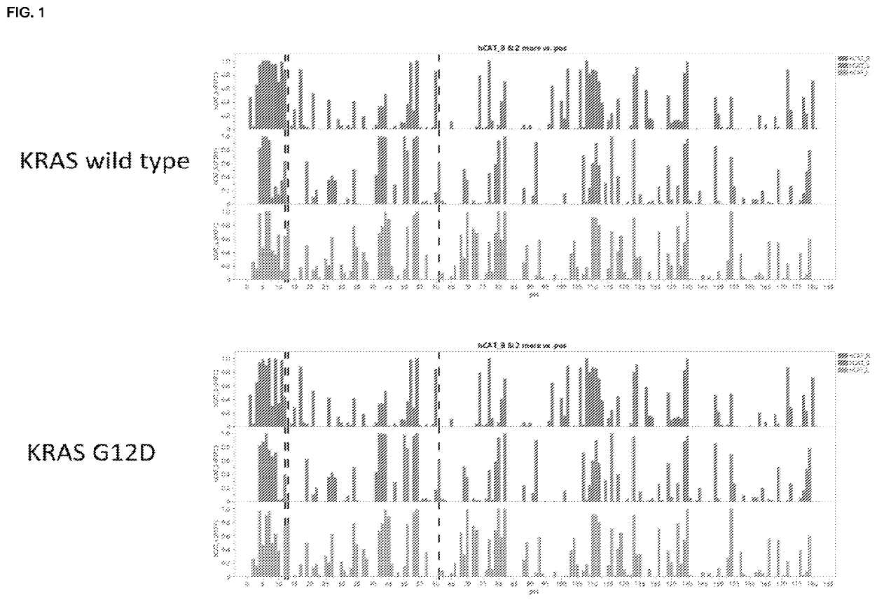

Cathepsins themselves have been shown to be antigenic and capable of generating antibody responses in the context of parasitic infection. Epitope mapping of human cathepsin L, B and S shown in FIG. 1 shows distinct and different linear B cell epitopes that would enable antibody targeting of cathepsin, either by standard tetrameric antibodies or subcomponents such as scFV. This is an approach which could enable neutralization of cathepsin or the targeting of other inhibitors to tumor cells with high upregulation of cathepsin. Given the relative lack of sequence conservation among these cathepsins a high degree of specificity would be expected. Sequences for cathepsin B, L and S are shown in Tables 1 and 2.

TABLE 1
Sequences of Cathepsin B, L and S.

| | |
|---|---|
| SEQ | P07858 CATB_HUMAN Cathepsin B |
| ID | MWQLWASLCCLLVLANARSRPSFHPLSDELVNYVNKRNTTWQAGHNFYNVDMSYLKRLCG |
| NO.: | TFLGGPKPPQRVMFTEDLKLPASFDAREQWPQCPTIKEIRDQGSCGSCWAFGAVEAISDR |
| 323 | ICIHTNAHVSVEVSAEDLLTCCGSMCGDGCNGGYPAEAWNFWTRKGLVSGGLYESHVGCR |
| | PYSIPPCEHHVNGSRPPCTGEGDTPKCSKICEPGYSPTYKQDKHYGYNSYSVSNSEKDIM |
| | AEIYKNGPVEGAFSVYSDFLLYKSGVYQHVTGEMMGGHAIRILGWGVENGTPYWLVANSW |
| | NTDWGDNGFFKILRGQDHCGIESEVVAGIPRTDQYWEKI |
| SEQ | P07711CATL1_HUMAN Procathepsin L |
| ID | MNPTLILAAFCLGIASATLTFDHSLEAQWTKWKAMHNRLYGMNEEGWRRAVWEKNMKMIE |
| NO.: | LHNQEYREGKHSFTMAMNAFGDMTSEEFRQVMNGFQNRKPRKGKVFQEPLFYEAPRSVDW |
| 324 | REKGYVTPVKNQGQCGSCWAFSATGALEGQMFRKTGRLISLSEQNLVDCSGPQGNEGCNG |
| | GLMDYAFQYVQDNGGLDSEESYPYEATEESCKYNPKYSVANDTGFVDIPKQEKALMKAVA |
| | TVGPISVAIDAGHESFLFYKEGIYFEPDCSSEDMDHGVLVVGYGFESTESDNNKYWLVKN |
| | SWGEEWGMGGYVKMAKDRRNHCGIASAASYPTV |
| SEQ | P25774 CATS_HUMAN Cathepsin S |
| ID | MKRLVCVLLVCSSAVAQLHKDPTLDHHWHLWKKTYGKQYKEKNEEAVRRLIWEKNLKFVM |
| NO.: | LHNLEHSMGMHSYDLGMNHLGDMTSEEVMSLMSSLRVPSQWQRNITYKSNPNRILPDSVD |
| 325 | WREKGCVTEVKYQGSCGACWAFSAVGALEAQLKLKTGKLVSLSAQNLVDCSTEKYGNKGC |
| | NGGFMTTAFQYIIDNKGIDSDASYPYKAMDQKCQYDSKYRAATCSKYTELPYGREDVLKE |
| | AVANKGPVSVGVDARHPSFFLYRSGVYYEPSCTQNVNHGVLVVGYGDLNGKEYWLVKNSW |
| | GHNFGEEGYIRMARNKGNHCGIASFPSYPEI |

TABLE 2
B cell epitopes in cathepsins

| | | |
|---|---|---|
| Cathepsin B | NARSRPSFHPLSDEL | SEQ ID NO.: 326 |
| | KRNTTWQAGHNFY | SEQ ID NO.: 327 |
| | LGGPKPPQRVMFTED | SEQ ID NO.: 328 |
| | IRDQGSCGSCWAF | SEQ ID NO.: 329 |
| | DGCNGGYPAEAWNFW | SEQ ID NO.: 330 |
| | VNGSRPPCTGEGDTPKCSKICEPG | SEQ ID NO.: 331 |
| | EPGYSPTYKQDKHYGYN | SEQ ID NO.: 332 |
| | YSVSNSEKDIMAEIY | SEQ ID NO.: 333 |
| Cathepsin L | NQEYREGKHSFTMA | SEQ ID NO.: 334 |
| | FQNRKPRKGKVFQEPLF | SEQ ID NO.: 335 |
| | KNQGQCGSCWAF | SEQ ID NO.: 336 |
| | DCSGPQGNEGCNGGLMDYA | SEQ ID NO.: 337 |
| | QDNGGLDSEESYPYEATEESCKYN | SEQ ID NO.: 338 |
| | CSSEDMDHGVLVV | SEQ ID NO.: 339 |
| | GFESTESDNNKYWLVKN | SEQ ID NO.: 340 |
| Cathepsin S | TYGKQYKEKNEEAVRRLIWE | SEQ ID NO.: 341 |
| | YKSNPNRILPDSVDW | SEQ ID NO.: 342 |
| | CSTEKYGNKGCNGGFMTTA | SEQ ID NO.: 343 |
| | NKGIDSDASYPYKAMDQK | SEQ ID NO.: 344 |
| | VANKGPVSVGVDARH | SEQ ID NO.: 345 |

In a further embodiment an antibody directed to a tumor upregulated protein, including for instance, as a non-limiting examples, brevican, EGFR, or a tumor associated antigen such as CEA or MAGEA1, and used to target a cathepsin inhibitor fused to or conjugated to that antibody to a particular tumor site. The antibody in this instance may be a standard tetrameric immunoglobulin or a sub-component such as a scFV.

### Combination Interventions.

In some embodiments the selected cathepsin inhibitor may be administered parenterally to the affected subject, either by injection or orally. In other embodiments the administration may be intratumorally, when it is desirable to apply the cathepsin inhibitor to the affected tumor cells. In other instances, for instance in a skin tumor the cathepsin inhibitor may be applied topically. In each case a pharmaceutically acceptable carrier may be used to facilitate delivery. Administration of the cathepsin inhibitor may be a standalone intervention, may include repeated doses or may be contemporaneous with administration of a neoantigen vaccine. It may be accompanied by or followed by the administration of a further immunomodulatory or immunotherapy intervention such as a checkpoint inhibitor.

Suitable neoantigen vaccines may be synthesized based on specific neoepitopes present in a subject or the vaccine may be prepared using common neoepitopes that regularly present in subjects. In some preferred embodiments, the vaccine is a peptide or polypeptide vaccine. In other preferred embodiments, the vaccine in an RNA vaccine. In still other preferred embodiments, the vaccine in a DNA vaccine. Suitable nucleic acid vaccines may be designed, for example, as described of US patent publications US20200254086, US20220152178, US20180369419, and/or US20210268086, each of which is incorporated herein by reference in its entirety. Further delivery vehicles may be employed to deliver the neoepitope vaccine including but not limited to viral or virus like particles.

A nucleic acid vaccine (e.g., RNA or DNA vaccines) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide. A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise.

Examples of naturally-occurring nucleotides used for the production of RNA, e.g., in a vaccine as provided herein include adenosine triphosphate (ATP), guanosine triphosphate (GTP), cytidine triphosphate (CTP), uridine triphosphate (UTP), and 5-methyluridine triphosphate (m5UTP). In some embodiments, adenosine diphosphate (ADP), guanosine diphosphate (GDP), cytidine diphosphate (CDP), and/or uridine diphosphate (UDP) are used.

Modified nucleotides may include modified nucleobases. For example, an RNA may include a modified nucleobase selected from pseudouridine (Y′), 1-methylpseudouridine (m1Y′), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, an RNA transcript (e.g., mRNA transcript) includes a combination of at least two (e.g., 2, 3, 4 or more) of the foregoing modified nucleobases. Other modifications include, but are not limited to, incorporation of fluorescently-labelled nucleobases.

Modified RNA also includes locked RNAs. Locked nucleic acid (LNA) (also known as 2′-0,4′-C-methylene-bridged nucleic acid (2′,4′-BNA)) are artificial nucleic acid derivatives. LNA contains a methylene bridge connecting the 2′-O with the 4′-C position in the furanose ring, which enables it to form a strictly N-type conformation that offers high binding affinity against complementary RNA. Representative U.S. Patents that teach the preparation of locked nucleic acid (LNA) include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety. Additional modified RNA molecules are described in U.S. Pat. No. 10,925,935, which is incorporated by reference herein in its entirety.

Suitable checkpoint inhibitors that may be used in conjunction with cathepsin inhibitors as described herein include, but are not limited to, PD-1 inhibitors (e.g., Nivolumab, Pembrolizumab, Dostarlimab and Cemiplimab), PD-L1 inhibitors (Atezolizumab, Avelumab, Durvalumab), CTLA-4 inhibitors (Ipilimumab, Tremelimumab), and LAG-3 inhibitors (Retalimab). Other checkpoint inhibitors in development, and which may be utilized in the present invention, include, but are not limited to, LAG525 (IMP701), REGN3767 (R3767), BI 754,091, tebotelimab (MGD013), eftilagimod alpha (IMP321), FS118, MBG453, Sym023, TSR-022, MGC018, FPA150, EOS100850, AB928, CPI-006, Monalizumab, COM701, CM24, NE)-201, Defactnib, PF-04136309, MSC-1, Hu5F9-G4 (5F9), ALX148, TTI-662, RRx-001, Lacnotuzumab (MCS110), LY3022855, SNDX-6352, emactuzumab (RG7155), pexidartinib (PLX3397), CAN04, Canakinumab (ACZ885), BMS-986253, Pepinemab (VX15/2503), Trebananib, FP-1305, Enapotamab vedotin (EnaV), and Bavituximab. These inhibitors may be used alone or in combination. In other embodiments, the checkpoint inhibitors may be used in combination with additional targeted therapeutic agents, for example, Axitinib, Cabozantinib, Levantinib, Cobimetinib, Vemurafenib, or Bevacizumab).

### Detection of Mutations

Mutations in Ras proteins or other cancer proteins may be detected by methods which are well established in the art. Suitable assays for detection and identification of tumor mutations include, but are not limited to, Taqman® assays (Applied Biosystems, Inc.), pyrosequencing, fluorescence resonance energy transfer (FRET)-based cleavage assays, fluorescent polarization, denaturing high performance liquid chromatography (DHPLC), mass spectrometry, and polynucleotides having fluorescent or radiological tags used in amplification and sequencing, and NextGen sequencing.

The present invention is not limited to particular methods of detecting the recited mutations. Markers may be detected as DNA (e.g., cDNA), RNA (e.g., mRNA), or protein.

In some embodiments, nucleic acid sequencing methods (sequencing assays) are utilized for detection. In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92:255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are suitable, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55:641-658, 2009; MacLean et al., Nature Rev. Microbiol., 7:287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In some embodiments, hybridization methods (hybridization assays) are utilized. Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with radio-, fluorescent- or antigen-labeled bases is localized and quantitated in the tissue using autoradiography, fluorescence microscopy or immunohistochemistry. ISH can also use two or more probes, labeled with radioactivity or the other non-radioactive labels, to simultaneously detect two or more transcripts.

In some embodiments, markers are detected using fluorescence in situ hybridization (FISH). The preferred FISH assays for methods of embodiments of the present disclosure utilize bacterial artificial chromosomes (BACs). These have been used extensively in the human genome sequencing project (see Nature 409:953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

Different kinds of biological assays are called microarrays including, but not limited to: microarrays (e.g., cDNA microarrays and oligonucleotide microarrays); protein microarrays; tissue microarrays; transfection or cell microarrays; chemical compound microarrays; and antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink-jet printing; or electrochemistry on microelectrode arrays.

Southern and Northern blotting may be used to detect specific DNA or RNA sequences, respectively. In these techniques DNA or RNA is extracted from a sample, fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

In some embodiments, marker sequences are amplified (amplification assays) prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

In some embodiments, quantitative evaluation of the amplification process in real-time is performed. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe: target duplexes in a test sample in the presence of unhybridized molecular torches. Molecular torches and a variety of types of interacting label pairs, including fluorescence resonance energy transfer (FRET) labels, are disclosed in, for example U.S. Pat. Nos. 6,534,274 and 5,776,782, each of which is herein incorporated by reference in its entirety.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed, for example, in U.S. Pat. Nos. 5,925,517 and 6,150,097, herein incorporated by reference in its entirety.

The cancer marker genes described herein may be detected as proteins using a variety of protein techniques known to those of ordinary skill in the art, including but not limited to, protein sequencing and immunoassays.

Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

Illustrative non-limiting examples of immunoassays include, but are not limited to: immunoprecipitation; Western blot; ELISA; immunohistochemistry; immunocytochemistry; flow cytometry; and immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

In some embodiments, a computer-based analysis program is used to translate the raw data generated by the detection assay (e.g., levels of the recited markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication system). Once received by the profiling service, the sample is processed and a profile is produced (i.e., marker levels) specific for the diagnostic or prognostic information desired for the subject.

The profile data is then prepared in a format suitable for interpretation by a treating clinician. For example, rather than providing raw data, the prepared format may represent a diagnosis or risk assessment (e.g., level of markers) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research. For example, the data may be used to further optimize the inclusion or elimination of markers as useful indicators of a particular condition or stage of disease or as a companion diagnostic to determine a treatment course of action.

Accordingly, methods described herein find use in determining a treatment course of action for a subject diagnosed with cancer. For example, in some embodiments, the patients stratified into a group where treatment with a cathepsin inhibitor in indicated.

### EXAMPLES

### Example 1: Cathepsin Profile of Oncogenes

Methods were previously developed to predict the cleavage of any peptide octomer at its central dimer site (42, 43) (U.S. Pat. No. 11,069,427 incorporated herein by reference). Briefly, these algorithms were developed using the following steps. Multiple chemical and physical properties reported amino acids were used to derive sets of principal components to provide proxies for each amino acid that encompass their variables. Drawing on large sets of experimentally determined peptide cleavage events (39, 44) for each cathepsin B, Lor S, sets of octomer peptides which were cleaved and sets which were uncleaved were used to train a classifier and generate an ensemble of predictive equations to predict cleavage or non-cleavage. These were further refined by bagging (bootstrap aggregation) repetition on multiple random subsets of the experimental dataset. To generate a cathepsin cleavage probability profile of a protein of interest, in the present instance a mutated tumor protein, each sequential octomer in the protein of interest is subjected to multiple repetitions of the ensemble of equations which in effect vote on whether the octomer is cleaved at its central dimer or not. The output is characterized as a probability score of 0-100% probability for each possible dimer in the 9mer that constitutes a potential neoepitope bound by a MHC I or the central 9mer of a 15mer bound by a MHC II. The probability of a mutated neoepitope being cleaved may be expressed according to the individual dimer cleavage probability or as the aggregate of cleavage probabilities across the neoepitope. An 80% probability of cleavage is considered a high score, together with any higher predicted probability.

In the context of a peptide that comprises a mutant amino acid we consider each 9mer that comprises that mutant. An MHC class I 9mer is comprised of eight potential scissile bonds. Cleavage of any these bonds will result in a loss of exposure of the TCEM within that 9mer to a T cell. Thus, a quantitative scoring metric for each TCEM pentamer is a summation of the number of scissile bonds in the peptide that are predicted to be cleaved by the enzyme with a probability of cleavage greater than a threshold. In practice a threshold of 0.8 (80%) is used and a maximum score of 8 occurs when all bonds have a high probability of being cleaved.

$$Score = {\sum }_{i = 1}^{8} ( cleavage prob scissle bond ( i ) \geq threshold )$$

By applying these predictive algorithms it is possible to draw a cathepsin profile for any protein showing the probability of cleavage at any amino acid dimer of interest in the protein, by cathepsin L, S or B and to derive a cleavage probability score for each 9mer that may play a role in tumor mutation recognition. When applied to a mutated tumor protein of interest this allows a prediction of whether a peptide comprising a mutant amino acid is likely to be excised as a peptide of suitable size for MHC binding and presentation and exposure of the mutant amino acid to the TCR, while in a further embodiment it predicts whether the peptide comprising the mutant amino acid is retained intact to allow presentation.

### Example 2: Cathepsin Profile of Ras Proteins

Analysis of the probability of cathepsin cleavage was conducted on KRAS, NRAS and HRAS. As the sequences for amino acid position 1-86 are identical and the hotspots for high frequency mutation are at positions G12, G13 and Q61, the figures and other comments herein are for KRAS but are equally applicable to the other two proteins.

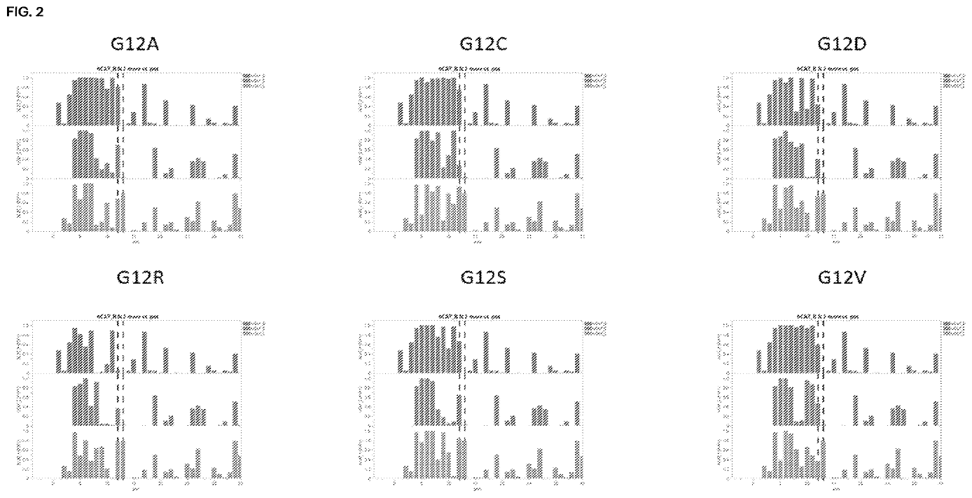

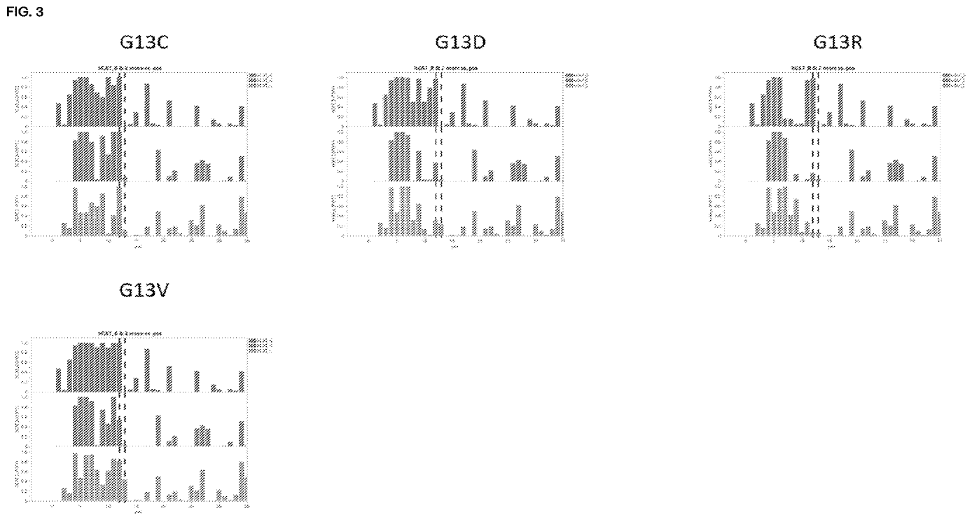

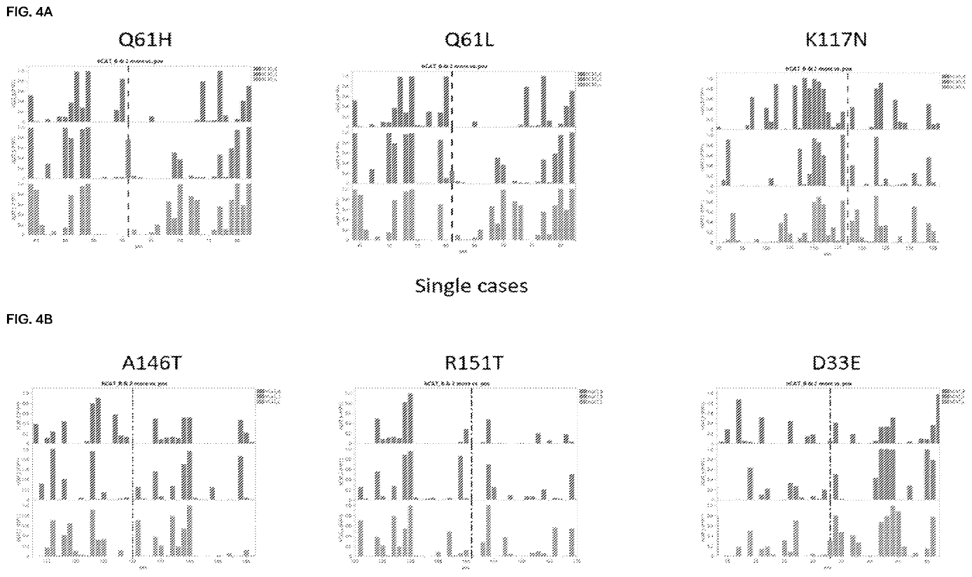

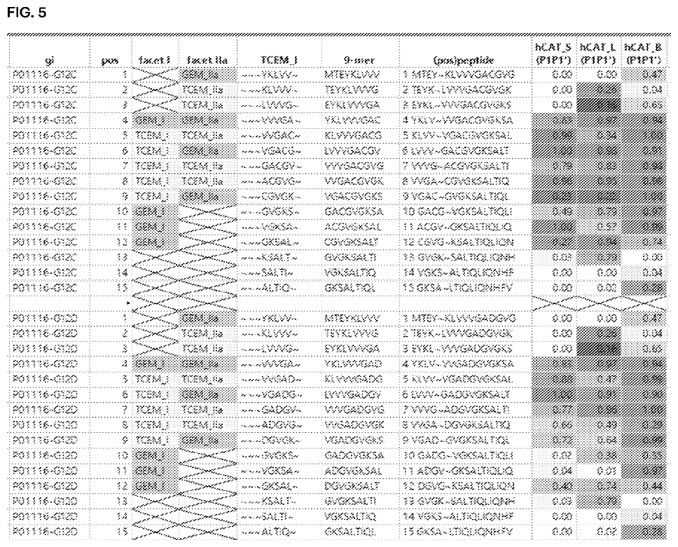

FIG. 1 shows the probability of cathepsin cleavage at each dimer position in the wildtype (unmutated) KRAS protein and the same pattern for the G12D mutant of KRAS. FIGS. 2, 3 and 4A show the cleavage pattern around G12, G13 and Q61 for the most common mutants. It will be noted that the vertical dashed lines indicate the mutant positions and the highest cleavage probability occurs on the N terminal side of the mutant position. FIG. 4B provides examples of less common KRAS mutants which exhibit a low probability pattern of cathepsin cleavage.

Table 3 shows the Cathepsin B cleavage probabilities by dimer position for two of the common KRAS mutations. Cleavage anywhere in the 9mer will impact the binding and TCR engagement of the 9mer. The same data but including all three cathepsins is shown in FIG. 5.

TABLE 3

| | | | SEQ ID | Cleavage | |
|---|---|---|---|---|---|
| gi | facet I | 9-mer | NO.: | position | CAT_B |
| P01116-G12C | GEM_I | YKLVVVGAC | 65 | YKLV~VVGAC | 0.94 |
| P01116-G12C | TCEM_I | KLVVVGACG | 66 | KLVV~VGACG | 1.00 |
| P01116-G12C | TCEM_I | LVVVGACGV | 67 | LVVV~GACGV | 0.91 |
| P01116-G12C | TCEM_I | VVVGACGVG | 68 | VVVG~ACGVG | 0.98 |
| P01116-G12C | TCEM_I | VVGACGVGK | 69 | VVGA~CGVGK | 0.98 |
| P01116-G12C | TCEM_I | VGACGVGKS | 70 | VGAC~GVGKS | 1.00 |
| P01116-G12C | GEM_I | GACGVGKSA | 71 | GACG~VGKSA | 0.97 |
| P01116-G12C | GEM_I | ACGVGKSAL | 72 | ACGV~GKSAL | 0.99 |
| P01116-G12C | GEM_I | CGVGKSALT | 73 | CGVG~KSALT | 0.74 |
| P01116-G12D | GEM_I | YKLVVVGAD | 74 | YKLV~VVGAD | 0.94 |
| P01116-G12D | TCEM_I | KLVVVGADG | 75 | KLVV~VGADG | 0.99 |
| P01116-G12D | TCEM_I | LVVVGADGV | 76 | LVVV~GADGV | 0.90 |
| P01116-G12D | TCEM_I | VVVGADGVG | 77 | VVVG~ADGVG | 1.00 |
| P01116-G12D | TCEM_I | VVGADGVGK | 78 | VVGA~DGVGK | 0.29 |
| P01116-G12D | TCEM_I | VGADGVGKS | 79 | VGAD~GVGKS | 0.99 |
| P01116-G12D | GEM_I | GADGVGKSA | 80 | GADG~VGKSA | 0.35 |
| P01116-G12D | GEM_I | ADGVGKSAL | 81 | ADGV~GKSAL | 0.97 |
| P01116-G12D | GEM_I | DGVGKSALT | 82 | DGVG~KSALT | 0.44 |

Mutant amino acid is boldfaced. CAT_B indicates the probability of cleavage by cathepsin B at the site shown

Table 4 shows the T cell exposed motifs comprising mutant amino acids in the KRAS mutation hotspots which may be affected by cleavage. TCEM I are those which would be exposed by an MHC I to a CD8+ cell. TCEM II are the discontinuous T cell exposed motifs which would be exposed when bound by an MHC II to a CD4+ T cell. Conversely if a cathepsin B inhibitor is active these TCEM motifs are the “rescued” neoantigens which are otherwise not presented to T cells and may now elicit an active cytotoxic response.

The peptides which encompass these T cell motifs in the common mutated KRAS proteins are shown in Table 5 and 6. As previously noted at these positions the sequences of NRAS and HRAS are identical.

TABLE 4
T cell exposed motifs comprising mutant amino acids in KRAS

| | | | SEQ ID | | | SEQ |
|---|---|---|---|---|---|---|
| | | | NO.: | | | ID NO.: |
| gi | Pos I | TCEM_I | TCEM I | Pos II | TCEM_II | TCEM II |
| P01116-G12A | 5 | ~~~VVGAA~ | 1 | 2 | LV~V~AA | 155 |
| P01116-G12A | 6 | ~~~VGAAG~ | 2 | 3 | VV~G~AG | 156 |
| P01116-G12A | 7 | ~~~GAAGV~ | 3 | 5 | VG~A~VG | 157 |
| P01116-G12A | 8 | ~~~AAGVG~ | 4 | 7 | AA~V~KS | 158 |
| P01116-G12A | 9 | ~~~AGVGK~ | 5 | 8 | AG~G~SA | 159 |
| P01116-G12C | 5 | ~~~VVGAC~ | 6 | 2 | LV~V~AC | 160 |
| P01116-G12C | 6 | ~~~VGACG~ | 7 | 3 | VV~G~CG | 161 |
| P01116-G12C | 7 | ~~~GACGV~ | 8 | 5 | VG~C~VG | 162 |
| P01116-G12C | 8 | ~~~ACGVG~ | 9 | 7 | AC~V~KS | 163 |
| P01116-G12C | 9 | ~~~CGVGK~ | 10 | 8 | CG~G~SA | 164 |
| P01116-G12D | 5 | ~~~VVGAD~ | 11 | 2 | LV~V~AD | 165 |
| P01116-G12D | 6 | ~~~VGADG~ | 12 | 3 | VV~G~DG | 166 |
| P01116-G12D | 7 | ~~~GADGV~ | 13 | 5 | VG~D~VG | 167 |
| P01116-G12D | 8 | ~~~ADGVG~ | 14 | 7 | AD~V~KS | 168 |
| P01116-G12D | 9 | ~~~DGVGK~ | 15 | 8 | DG~G~SA | 169 |
| P01116-G12R | 5 | ~~~VVGAR~ | 16 | 2 | LV~V~AR | 170 |
| P01116-G12R | 6 | ~~~VGARG~ | 17 | 3 | VV~G~RG | 171 |
| P01116-G12R | 7 | ~~~GARGV~ | 18 | 5 | VG~R~VG | 172 |
| P01116-G12R | 8 | ~~~ARGVG~ | 19 | 7 | AR~V~KS | 173 |
| P01116-G12R | 9 | ~~~RGVGK~ | 20 | 8 | RG~G~SA | 174 |
| P01116-G12S | 5 | ~~~VVGAS~ | 21 | 2 | LV~V~AS | 175 |
| P01116-G12S | 6 | ~~~VGASG~ | 22 | 3 | VV~G~SG | 176 |
| P01116-G12S | 7 | ~~~GASGV~ | 23 | 5 | VG~S~VG | 177 |
| P01116-G12S | 8 | ~~~ASGVG~ | 24 | 7 | AS~V~KS | 178 |
| P01116-G12S | 9 | ~~~SGVGK~ | 25 | 8 | SG~G~SA | 179 |
| P01116-G12V | 5 | ~~~VVGAV~ | 26 | 2 | LV~V~AV | 180 |
| P01116-G12V | 6 | ~~~VGAVG~ | 27 | 3 | VV~G~VG | 181 |
| P01116-G12V | 7 | ~~~GAVGV~ | 28 | 5 | VG~V~VG | 182 |
| P01116-G12V | 8 | ~~~AVGVG~ | 29 | 7 | AV~V~KS | 183 |
| P01116-G12V | 9 | ~~~VGVGK~ | 30 | 8 | VG~G~SA | 184 |
| P01116-G13C | 6 | ~~~VGAGC~ | 31 | 3 | VV~G~GC | 185 |
| P01116-G13C | 7 | ~~~GAGCV~ | 32 | 4 | VV~A~CV | 186 |
| P01116-G13C | 8 | ~~~AGCVG~ | 33 | 6 | GA~C~GK | 187 |
| P01116-G13C | 9 | ~~~GCVGK~ | 34 | 8 | GC~G~SA | 188 |
| P01116-G13C | 10 | ~~~CVGKS~ | 35 | 9 | CV~K~AL | 189 |
| P01116-G13D | 6 | ~~~VGAGD~ | 36 | 3 | VV~G~GD | 190 |
| P01116-G13D | 7 | ~~~GAGDV~ | 37 | 4 | VV~A~DV | 191 |
| P01116-G13D | 8 | ~~~AGDVG~ | 38 | 6 | GA~D~GK | 192 |
| P01116-G13D | 9 | ~~~GDVGK~ | 39 | 8 | GD~G~SA | 193 |
| P01116-G13D | 10 | ~~~DVGKS~ | 40 | 9 | DV~K~AL | 194 |
| P01116-G13R | 6 | ~~~VGAGR~ | 41 | 3 | VV~G~GR | 195 |
| P01116-G13R | 7 | ~~~GAGRV~ | 42 | 4 | VV~A~RV | 196 |
| P01116-G13R | 8 | ~~~AGRVG~ | 43 | 6 | GA~R~GK | 197 |
| P01116-G13R | 9 | ~~~GRVGK~ | 44 | 8 | GR~G~SA | 198 |
| P01116-G13R | 10 | ~~~RVGKS~ | 45 | 9 | RV~K~AL | 199 |
| P01116-G13V | 6 | ~~~VGAGV~ | 46 | 3 | VV~G~GV | 200 |
| P01116-G13V | 7 | ~~~GAGVV~ | 47 | 4 | VV~A~VV | 201 |
| P01116-G13V | 8 | ~~~AGVVG~ | 48 | 6 | GA~V~GK | 202 |
| P01116-G13V | 9 | ~~~GVVGK~ | 49 | 8 | GV~G~SA | 203 |
| P01116-G13V | 10 | ~~~VVGKS~ | 50 | 9 | VV~K~AL | 204 |
| P01116-Q61H | 54 | ~~~DTAGH~ | 51 | 51 | IL~T~GH | 205 |
| P01116-Q61H | 55 | ~~~TAGHE~ | 52 | 52 | LD~A~HE | 206 |
| P01116-Q61H | 56 | ~~~AGHEE~ | 53 | 54 | TA~H~EY | 207 |
| P01116-Q61H | 57 | ~~~GHEEY~ | 54 | 56 | GH~E~SA | 208 |
| P01116-Q61H | 58 | ~~~HEEYS~ | 55 | 57 | HE~Y~AM | 209 |

TABLE 5
KRAS mutated 9 mer peptides with high probability of cathepsin cleavage.

| gi | pos | facet I | 9-mer | SEQ ID NO.: |
|---|---|---|---|---|
| P01116-G12A | 4 | GEM_I | YKLVVVGAA | 56 |
| P01116-G12A | 5 | TCEM_I | KLVVVGAAG | 57 |
| P01116-G12A | 6 | TCEM_I | LVVVGAAGV | 58 |
| P01116-G12A | 7 | TCEM_I | VVVGAAGVG | 59 |
| P01116-G12A | 8 | TCEM_I | VVGAAGVGK | 60 |
| P01116-G12A | 9 | TCEM_I | VGAAGVGKS | 61 |
| P01116-G12A | 10 | GEM_I | GAAGVGKSA | 62 |
| P01116-G12A | 11 | GEM_I | AAGVGKSAL | 63 |
| P01116-G12A | 12 | GEM_I | AGVGKSALT | 64 |
| P01116-G12C | 4 | GEM_I | YKLVVVGAC | 65 |
| P01116-G12C | 5 | TCEM_I | KLVVVGACG | 66 |
| P01116-G12C | 6 | TCEM_I | LVVVGACGV | 67 |
| P01116-G12C | 7 | TCEM_I | VVVGACGVG | 68 |
| P01116-G12C | 8 | TCEM_I | VVGACGVGK | 69 |
| P01116-G12C | 9 | TCEM_I | VGACGVGKS | 70 |
| P01116-G12C | 10 | GEM_I | GACGVGKSA | 71 |
| P01116-G12C | 11 | GEM_I | ACGVGKSAL | 72 |
| P01116-G12C | 12 | GEM_I | CGVGKSALT | 73 |
| P01116-G12D | 4 | GEM_I | YKLVVVGAD | 74 |
| P01116-G12D | 5 | TCEM_I | KLVVVGADG | 75 |
| P01116-G12D | 6 | TCEM_I | LVVVGADGV | 76 |
| P01116-G12D | 7 | TCEM_I | VVVGADGVG | 77 |
| P01116-G12D | 8 | TCEM_I | VVGADGVGK | 78 |
| P01116-G12D | 9 | TCEM_I | VGADGVGKS | 79 |
| P01116-G12D | 10 | GEM_I | GADGVGKSA | 80 |
| P01116-G12D | 11 | GEM_I | ADGVGKSAL | 81 |
| P01116-G12D | 12 | GEM_I | DGVGKSALT | 82 |
| P01116-G12R | 4 | GEM_I | YKLVVVGAR | 83 |
| P01116-G12R | 5 | TCEM_I | KLVVVGARG | 84 |
| P01116-G12R | 6 | TCEM_I | LVVVGARGV | 85 |
| P01116-G12R | 7 | TCEM_I | VVVGARGVG | 86 |
| P01116-G12R | 8 | TCEM_I | VVGARGVGK | 87 |
| P01116-G12R | 9 | TCEM_I | VGARGVGKS | 88 |
| P01116-G12R | 10 | GEM_I | GARGVGKSA | 89 |
| P01116-G12R | 11 | GEM_I | ARGVGKSAL | 90 |
| P01116-G12R | 12 | GEM_I | RGVGKSALT | 91 |
| P01116-G12S | 4 | GEM_I | YKLVVVGAS | 92 |
| P01116-G12S | 5 | TCEM_I | KLVVVGASG | 93 |
| P01116-G12S | 6 | TCEM_I | LVVVGASGV | 94 |
| P01116-G12S | 7 | TCEM_I | VVVGASGVG | 95 |
| P01116-G12S | 8 | TCEM_I | VVGASGVGK | 96 |
| P01116-G12S | 9 | TCEM_I | VGASGVGKS | 97 |
| P01116-G12S | 10 | GEM_I | GASGVGKSA | 98 |
| P01116-G12S | 11 | GEM_I | ASGVGKSAL | 99 |
| P01116-G12S | 12 | GEM_I | SGVGKSALT | 100 |
| P01116-G12V | 4 | GEM_I | YKLVVVGAV | 101 |
| P01116-G12V | 5 | TCEM_I | KLVVVGAVG | 102 |
| P01116-G12V | 6 | TCEM_I | LVVVGAVGV | 103 |
| P01116-G12V | 7 | TCEM_I | VVVGAVGVG | 104 |
| P01116-G12V | 8 | TCEM_I | VVGAVGVGK | 105 |
| P01116-G12V | 9 | TCEM_I | VGAVGVGKS | 106 |
| P01116-G12V | 10 | GEM_I | GAVGVGKSA | 107 |
| P01116-G12V | 11 | GEM_I | AVGVGKSAL | 108 |
| P01116-G12V | 12 | GEM_I | VGVGKSALT | 109 |
| P01116-G13C | 5 | GEM_I | KLVVVGAGC | 110 |
| P01116-G13C | 6 | TCEM_I | LVVVGAGCV | 111 |
| P01116-G13C | 7 | TCEM_I | VVVGAGCVG | 112 |
| P01116-G13C | 8 | TCEM_I | VVGAGCVGK | 113 |
| P01116-G13C | 9 | TCEM_I | VGAGCVGKS | 114 |
| P01116-G13C | 10 | TCEM_I | GAGCVGKSA | 115 |
| P01116-G13C | 11 | GEM_I | AGCVGKSAL | 116 |
| P01116-G13C | 12 | GEM_I | GCVGKSALT | 117 |
| P01116-G13C | 13 | GEM_I | CVGKSALTI | 118 |
| P01116-G13D | 5 | GEM_I | KLVVVGAGD | 119 |
| P01116-G13D | 6 | TCEM_I | LVVVGAGDV | 120 |
| P01116-G13D | 7 | TCEM_I | VVVGAGDVG | 121 |
| P01116-G13D | 8 | TCEM_I | VVGAGDVGK | 122 |
| P01116-G13D | 9 | TCEM_I | VGAGDVGKS | 123 |
| P01116-G13D | 10 | TCEM_I | GAGDVGKSA | 124 |
| P01116-G13D | 11 | GEM_I | AGDVGKSAL | 125 |
| P01116-G13D | 12 | GEM_I | GDVGKSALT | 126 |
| P01116-G13D | 13 | GEM_I | DVGKSALTI | 127 |
| P01116-G13R | 5 | GEM_I | KLVVVGAGR | 128 |
| P01116-G13R | 6 | TCEM_I | LVVVGAGRV | 129 |
| P01116-G13R | 7 | TCEM_I | VVVGAGRVG | 130 |
| P01116-G13R | 8 | TCEM_I | VVGAGRVGK | 131 |
| P01116-G13R | 9 | TCEM_I | VGAGRVGKS | 132 |
| P01116-G13R | 10 | TCEM_I | GAGRVGKSA | 133 |
| P01116-G13R | 11 | GEM_I | AGRVGKSAL | 134 |
| P01116-G13R | 12 | GEM_I | GRVGKSALT | 135 |
| P01116-G13R | 13 | GEM_I | RVGKSALTI | 136 |
| P01116-G13V | 5 | GEM_I | KLVVVGAGV | 137 |
| P01116-G13V | 6 | TCEM_I | LVVVGAGVV | 138 |
| P01116-G13V | 7 | TCEM_I | VVVGAGVVG | 139 |
| P01116-G13V | 8 | TCEM_I | VVGAGVVGK | 140 |
| P01116-G13V | 9 | TCEM_I | VGAGVVGKS | 141 |
| P01116-G13V | 10 | TCEM_I | GAGVVGKSA | 142 |
| P01116-G13V | 11 | GEM_I | AGVVGKSAL | 143 |
| P01116-G13V | 12 | GEM_I | GVVGKSALT | 144 |
| P01116-G13V | 13 | GEM_I | VVGKSALTI | 145 |
| P01116-Q61H | 53 | GEM_I | LDILDTAGH | 146 |
| P01116-Q61H | 54 | TCEM_I | DILDTAGHE | 147 |
| P01116-Q61H | 55 | TCEM_I | ILDTAGHEE | 148 |
| P01116-Q61H | 56 | TCEM_I | LDTAGHEEY | 149 |
| P01116-Q61H | 57 | TCEM_I | DTAGHEEYS | 150 |
| P01116-Q61H | 58 | TCEM_I | TAGHEEYSA | 151 |
| P01116-Q61H | 59 | GEM_I | AGHEEYSAM | 152 |
| P01116-Q61H | 60 | GEM_I | GHEEYSAMR | 153 |
| P01116-Q61H | 61 | GEM_I | HEEYSAMRD | 154 |

“pos” indicated index amino acid position in protein.

TABLE 6
KRAS mutated 15 mer peptides with high probability of cathepsin cleavage

| gi | pos | facet II | peptide | SEQ ID NO.: |
|---|---|---|---|---|
| P01116-G12A | 1 | GEM_II | MTEYKLVVVGAAGVG | 210 |
| P01116-G12A | 2 | TCEM_II | TEYKLVVVGAAGVGK | 211 |
| P01116-G12A | 3 | TCEM_II | EYKLVVVGAAGVGKS | 212 |
| P01116-G12A | 4 | GEM_II | YKLVVVGAAGVGKSA | 213 |
| P01116-G12A | 5 | TCEM_II | KLVVVGAAGVGKSAL | 214 |
| P01116-G12A | 6 | GEM_II | LVVVGAAGVGKSALT | 215 |
| P01116-G12A | 7 | TCEM_II | VVVGAAGVGKSALTI | 216 |
| P01116-G12A | 8 | TCEM_II | VVGAAGVGKSALTIQ | 217 |
| P01116-G12A | 9 | GEM_II | VGAAGVGKSALTIQL | 218 |
| P01116-G12C | 1 | GEM_II | MTEYKLVVVGACGVG | 219 |
| P01116-G12C | 2 | TCEM_II | TEYKLVVVGACGVGK | 220 |
| P01116-G12C | 3 | TCEM_II | EYKLVVVGACGVGKS | 221 |
| P01116-G12C | 4 | GEM_II | YKLVVVGACGVGKSA | 222 |
| P01116-G12C | 5 | TCEM_II | KLVVVGACGVGKSAL | 223 |
| P01116-G12C | 6 | GEM_II | LVVVGACGVGKSALT | 224 |
| P01116-G12C | 7 | TCEM_II | VVVGACGVGKSALTI | 225 |
| P01116-G12C | 8 | TCEM_II | VVGACGVGKSALTIQ | 226 |
| P01116-G12C | 9 | GEM_II | VGACGVGKSALTIQL | 227 |
| P01116-G12D | 1 | GEM_II | MTEYKLVVVGADGVG | 228 |
| P01116-G12D | 2 | TCEM_II | TEYKLVVVGADGVGK | 229 |
| P01116-G12D | 3 | TCEM_II | EYKLVVVGADGVGKS | 230 |
| P01116-G12D | 4 | GEM_II | YKLVVVGADGVGKSA | 231 |
| P01116-G12D | 5 | TCEM_II | KLVVVGADGVGKSAL | 232 |
| P01116-G12D | 6 | GEM_II | LVVVGADGVGKSALT | 233 |
| P01116-G12D | 7 | TCEM_II | VVVGADGVGKSALTI | 234 |
| P01116-G12D | 8 | TCEM_II | VVGADGVGKSALTIQ | 235 |
| P01116-G12D | 9 | GEM_II | VGADGVGKSALTIQL | 236 |
| P01116-G12R | 1 | GEM_II | MTEYKLVVVGARGVG | 237 |
| P01116-G12R | 2 | TCEM_II | TEYKLVVVGARGVGK | 238 |
| P01116-G12R | 3 | TCEM_II | EYKLVVVGARGVGKS | 239 |
| P01116-G12R | 4 | GEM_II | YKLVVVGARGVGKSA | 240 |
| P01116-G12R | 5 | TCEM_II | KLVVVGARGVGKSAL | 241 |
| P01116-G12R | 6 | GEM_II | LVVVGARGVGKSALT | 242 |
| P01116-G12R | 7 | TCEM_II | VVVGARGVGKSALTI | 243 |
| P01116-G12R | 8 | TCEM_II | VVGARGVGKSALTIQ | 244 |
| P01116-G12R | 9 | GEM_II | VGARGVGKSALTIQL | 245 |
| P01116-G12S | 1 | GEM_II | MTEYKLVVVGASGVG | 246 |
| P01116-G12S | 2 | TCEM_II | TEYKLVVVGASGVGK | 247 |
| P01116-G12S | 3 | TCEM_II | EYKLVVVGASGVGKS | 248 |
| P01116-G12S | 4 | GEM_II | YKLVVVGASGVGKSA | 249 |
| P01116-G12S | 5 | TCEM_II | KLVVVGASGVGKSAL | 250 |
| P01116-G12S | 6 | GEM_II | LVVVGASGVGKSALT | 251 |
| P01116-G12S | 7 | TCEM_II | VVVGASGVGKSALTI | 252 |
| P01116-G12S | 8 | TCEM_II | VVGASGVGKSALTIQ | 253 |
| P01116-G12S | 9 | GEM_II | VGASGVGKSALTIQL | 254 |
| P01116-G12V | 1 | GEM_II | MTEYKLVVVGAVGVG | 255 |
| P01116-G12V | 2 | TCEM_II | TEYKLVVVGAVGVGK | 256 |
| P01116-G12V | 3 | TCEM_II | EYKLVVVGAVGVGKS | 257 |
| P01116-G12V | 4 | GEM_II | YKLVVVGAVGVGKSA | 258 |
| P01116-G12V | 5 | TCEM_II | KLVVVGAVGVGKSAL | 259 |
| P01116-G12V | 6 | GEM_II | LVVVGAVGVGKSALT | 260 |
| P01116-G12V | 7 | TCEM_II | VVVGAVGVGKSALTI | 261 |
| P01116-G12V | 8 | TCEM_II | VVGAVGVGKSALTIQ | 262 |
| P01116-G12V | 9 | GEM_II | VGAVGVGKSALTIQL | 263 |
| P01116-G13C | 2 | GEM_II | TEYKLVVVGAGCVGK | 264 |
| P01116-G13C | 3 | TCEM_II | EYKLVVVGAGCVGKS | 265 |
| P01116-G13C | 4 | TCEM_II | YKLVVVGAGCVGKSA | 266 |
| P01116-G13C | 5 | GEM_II | KLVVVGAGCVGKSAL | 267 |
| P01116-G13C | 6 | TCEM_II | LVVVGAGCVGKSALT | 268 |
| P01116-G13C | 7 | GEM_II | VVVGAGCVGKSALTI | 269 |
| P01116-G13C | 8 | TCEM_II | VVGAGCVGKSALTIQ | 270 |
| P01116-G13C | 9 | TCEM_II | VGAGCVGKSALTIQL | 271 |
| P01116-G13C | 10 | GEM_II | GAGCVGKSALTIQLI | 272 |
| P01116-G13D | 2 | GEM_II | TEYKLVVVGAGDVGK | 273 |
| P01116-G13D | 3 | TCEM_II | EYKLVVVGAGDVGKS | 274 |
| P01116-G13D | 4 | TCEM_II | YKLVVVGAGDVGKSA | 275 |
| P01116-G13D | 5 | GEM_II | KLVVVGAGDVGKSAL | 276 |
| P01116-G13D | 6 | TCEM_II | LVVVGAGDVGKSALT | 277 |
| P01116-G13D | 7 | GEM_II | VVVGAGDVGKSALTI | 278 |
| P01116-G13D | 8 | TCEM_II | VVGAGDVGKSALTIQ | 279 |
| P01116-G13D | 9 | TCEM_II | VGAGDVGKSALTIQL | 280 |
| P01116-G13D | 10 | GEM_II | GAGDVGKSALTIQLI | 281 |
| P01116-G13R | 2 | GEM_II | TEYKLVVVGAGRVGK | 282 |
| P01116-G13R | 3 | TCEM_II | EYKLVVVGAGRVGKS | 283 |
| P01116-G13R | 4 | TCEM_II | YKLVVVGAGRVGKSA | 284 |
| P01116-G13R | 5 | GEM_II | KLVVVGAGRVGKSAL | 285 |
| P01116-G13R | 6 | TCEM_II | LVVVGAGRVGKSALT | 286 |
| P01116-G13R | 7 | GEM_II | VVVGAGRVGKSALTI | 287 |
| P01116-G13R | 8 | TCEM_II | VVGAGRVGKSALTIQ | 288 |
| P01116-G13R | 9 | TCEM_II | VGAGRVGKSALTIQL | 289 |
| P01116-G13R | 10 | GEM_II | GAGRVGKSALTIQLI | 290 |
| P01116-G13V | 2 | GEM_II | TEYKLVVVGAGVVGK | 291 |
| P01116-G13V | 3 | TCEM_II | EYKLVVVGAGVVGKS | 292 |
| P01116-G13V | 4 | TCEM_II | YKLVVVGAGVVGKSA | 293 |
| P01116-G13V | 5 | GEM_II | KLVVVGAGVVGKSAL | 294 |
| P01116-G13V | 6 | TCEM_II | LVVVGAGVVGKSALT | 295 |
| P01116-G13V | 7 | GEM_II | VVVGAGVVGKSALTI | 296 |
| P01116-G13V | 8 | TCEM_II | VVGAGVVGKSALTIQ | 297 |
| P01116-G13V | 9 | TCEM_II | VGAGVVGKSALTIQL | 298 |
| P01116-G13V | 10 | GEM_II | GAGVVGKSALTIQLI | 299 |
| P01116-Q61H | 50 | GEM_II | TCLLDILDTAGHEEY | 300 |
| P01116-Q61H | 51 | TCEM_II | CLLDILDTAGHEEYS | 301 |
| P01116-Q61H | 52 | TCEM_II | LLDILDTAGHEEYSA | 302 |
| P01116-Q61H | 53 | GEM_II | LDILDTAGHEEYSAM | 303 |
| P01116-Q61H | 54 | TCEM_II | DILDTAGHEEYSAMR | 304 |
| P01116-Q61H | 55 | GEM_II | ILDTAGHEEYSAMRD | 305 |
| P01116-Q61H | 56 | TCEM_II | LDTAGHEEYSAMRDQ | 306 |
| P01116-Q61H | 57 | TCEM_II | DTAGHEEYSAMRDQY | 307 |
| P01116-Q61H | 58 | GEM_II | TAGHEEYSAMRDQYM | 308 |

“pos” indicated index amino acid position in protein.

The peptides and the T cell exposed motifs they comprise have the potential to become candidate neoantigens if cleavage is abrogated by a cathepsin inhibitor. Selection of peptide depends on the actual mutation in the individual subject and the subjects HLA and the binding of the peptide to the HLA.

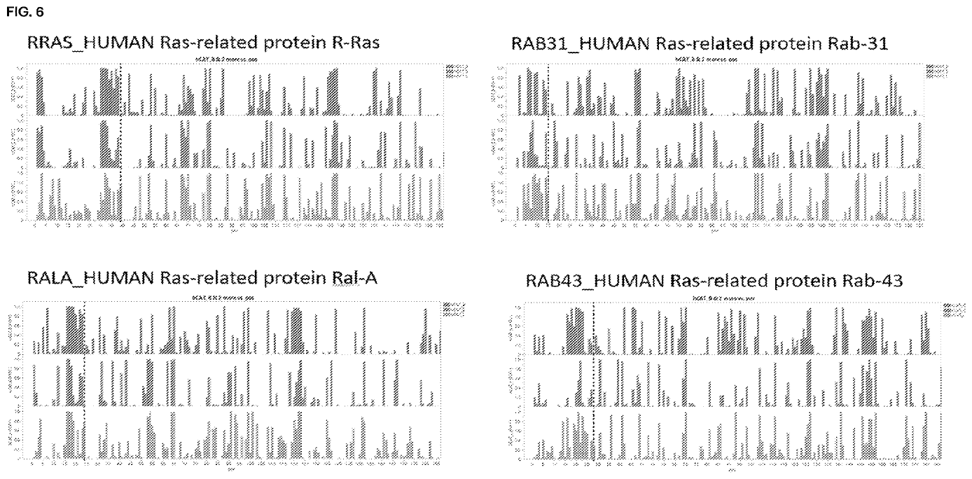

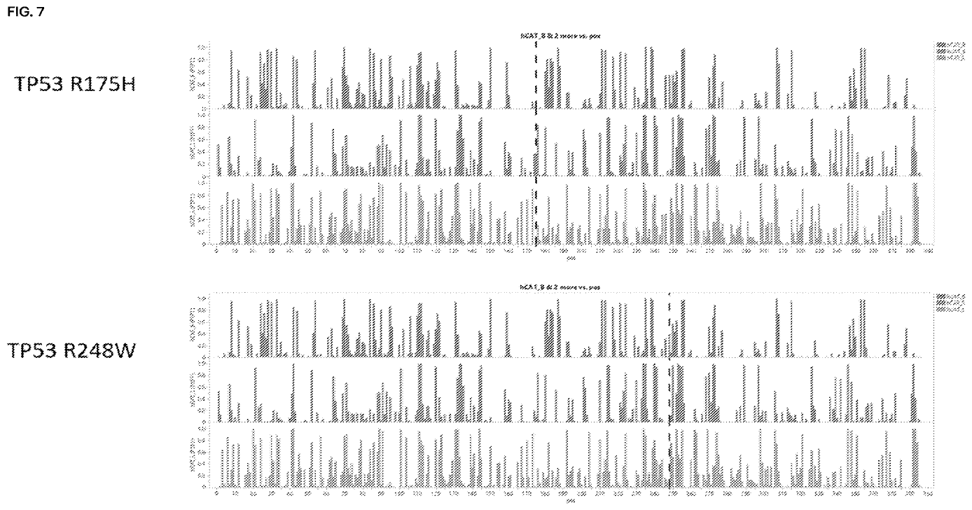

FIG. 6 shows that other GTPases and RAS like proteins have a similar density of high probability cleavage sites at positions that align to the positions 12 and 13 in KRAS.

### Example 3: Precursor Frequency Probability

The presence of clonal populations of T cells reactive to a given mutation is influenced by the number of matching epitopes which may have previously stimulated T cell clones. This includes epitopes in the human proteome and those in the microbiome and exogenous environment. As the motif which engages the TCR is a pentameric motif and a limited number of configurations of a pentamer can exist, the frequency of the pentameric motifs in the human proteome and a representative gastrointestinal microbiome were assessed as an index of motif frequency. Some mutations in tumor drivers escape immune surveillance because they comprise very rare T cell exposed motifs, as is the case for some common TP53 mutations (9). This is quite different from the common mutations of KRAS. As shown in Table 7, the T cell exposed motifs of the common KRAS mutations have high counts in the human proteome and in a large representative gastrointestinal microbiome. Table 7 illustrates the pattern of matching T cell motifs in the whole human proteome and microbiome for the G12 positions. Similarly high numbers of matching TCEM are found for G13 and Q61 mutants.

Amino acid motif frequencies, corresponding to the continuous and discontinuous pentameric configurations of TCEM, were determined in the Hg38 human proteome HUMAN_9606 retrieved from the UniProt repository and excluding immunoglobulins (78). The longest isoform of each protein was selected and used for extraction of the motif frequencies. Each protein in the dataset was broken into successive 15mers with a sliding window displaced by a single amino acid, as previously described (79). This dataset comprised approximately 11.63 million peptides. For the gastrointestinal microbiome reference dataset the same process was carried out for all open reading frames in the genomes of 67 bacterial species in 35 genera assembled from the NIH Human Microbiome Project Reference Genomes database (www.hmpdacc.org/HMRGD) (79, 80). The gastrointestinal microbiome reference dataset is about ten times larger than the human proteome, approximately 109 million peptides.

TABLE 7
KRAS mutant TCEM motifs and the count of corresponding pentamer motifs in human
proteome and gastrointestinal microbiome.

| gi | | | SEQ ID NO.: | Count in | Count in GI |
|---|---|---|---|---|---|
| | pos | | TCEM | proteome | microbiome |
| | | TCEM_I | | | |
| P01116-G12A | 5 | ~~~VVGAA~ | 1 | 22 | 310 |
| P01116-G12A | 6 | ~~~VGAAG~ | 2 | 56 | 285 |
| P01116-G12A | 7 | ~~~GAAGV~ | 3 | 47 | 451 |
| P01116-G12A | 8 | ~~~AAGVG~ | 4 | 43 | 442 |
| P01116-G12A | 9 | ~~~AGVGK~ | 5 | 20 | 402 |
| P01116-G12C | 5 | ~~~VVGAC~ | 6 | 3 | 23 |
| P01116-G12C | 6 | ~~~VGACG~ | 7 | 5 | 70 |
| P01116-G12C | 7 | ~~~GACGV~ | 8 | 3 | 106 |
| P01116-G12C | 8 | ~~~ACGVG~ | 9 | 3 | 103 |
| P01116-G12C | 9 | ~~~CGVGK~ | 10 | 5 | 79 |
| P01116-G12D | 5 | ~~~VVGAD~ | 11 | 6 | 156 |
| P01116-G12D | 6 | ~~~VGADG~ | 12 | 8 | 273 |
| P01116-G12D | 7 | ~~~GADGV~ | 13 | 18 | 425 |
| P01116-G12D | 8 | ~~~ADGVG~ | 14 | 10 | 124 |
| P01116-G12D | 9 | ~~~DGVGK~ | 15 | 0 | 103 |
| P01116-G12R | 5 | ~~~VVGAR~ | 16 | 14 | 102 |
| P01116-G12R | 6 | ~~~VGARG~ | 17 | 19 | 111 |
| P01116-G12R | 7 | ~~~GARGV~ | 18 | 22 | 121 |
| P01116-G12R | 8 | ~~~ARGVG~ | 19 | 14 | 82 |
| P01116-G12R | 9 | ~~~RGVGK~ | 20 | 11 | 124 |
| P01116-G12S | 5 | ~~~VVGAS~ | 21 | 20 | 185 |
| P01116-G12S | 6 | ~~~VGASG~ | 22 | 26 | 402 |
| P01116-G12S | 7 | ~~~GASGV~ | 23 | 23 | 390 |
| P01116-G12S | 8 | ~~~ASGVG~ | 24 | 24 | 336 |
| P01116-G12S | 9 | ~~~SGVGK~ | 25 | 30 | 556 |
| P01116-G12V | 5 | ~~~VVGAV~ | 26 | 29 | 385 |
| P01116-G12V | 6 | ~~~VGAVG~ | 27 | 23 | 223 |
| P01116-G12V | 7 | ~~~GAVGV~ | 28 | 16 | 302 |
| P01116-G12V | 8 | ~~~AVGVG~ | 29 | 26 | 237 |
| P01116-G12V | 9 | ~~~VGVGK~ | 30 | 10 | 129 |
| | | TCEMII | | | |
| P01116-G12A | 2 | LV~V~AA | 155 | 31 | 461 |
| P01116-G12A | 3 | VV~G~AG | 156 | 19 | 414 |
| P01116-G12A | 5 | VG~A~VG | 157 | 26 | 274 |
| P01116-G12A | 7 | AA~V~KS | 158 | 12 | 100 |
| P01116-G12A | 8 | AG~G~SA | 159 | 48 | 595 |
| P01116-G12C | 2 | LV~V~AC | 160 | 3 | 104 |
| P01116-G12C | 3 | VV~G~CG | 161 | 3 | 59 |
| P01116-G12C | 5 | VG~C~VG | 162 | 6 | 40 |
| P01116-G12C | 7 | AC~V~KS | 163 | 3 | 16 |
| P01116-G12C | 8 | CG~G~SA | 164 | 7 | 132 |
| P01116-G12D | 2 | LV~V~AD | 165 | 8 | 165 |
| P01116-G12D | 3 | VV~G~DG | 166 | 27 | 35 |
| P01116-G12D | 5 | VG~D~VG | 167 | 5 | 108 |
| P01116-G12D | 7 | AD~V~KS | 168 | 9 | 131 |
| P01116-G12D | 8 | DG~G~SA | 169 | 14 | 99 |
| P01116-G12R | 2 | LV~V~AR | 170 | 15 | 121 |
| P01116-G12R | 3 | VV~G~RG | 171 | 13 | 109 |
| P01116-G12R | 5 | VG~R~VG | 172 | 10 | 155 |
| P01116-G12R | 7 | AR~V~KS | 173 | 13 | 101 |
| P01116-G12R | 8 | RG~G~SA | 174 | 33 | 141 |
| P01116-G12S | 2 | LV~V~AS | 175 | 20 | 209 |
| P01116-G12S | 3 | VV~G~SG | 176 | 41 | 539 |
| P01116-G12S | 5 | VG~S~VG | 177 | 25 | 192 |
| P01116-G12S | 7 | AS~V~KS | 178 | 12 | 89 |
| P01116-G12S | 8 | SG~G~SA | 179 | 47 | 397 |
| P01116-G12V | 2 | LV~V~AV | 180 | 23 | 352 |
| P01116-G12V | 3 | VV~G~VG | 181 | 23 | 317 |
| P01116-G12V | 5 | VG~V~VG | 182 | 11 | 278 |
| P01116-G12V | 7 | AV~V~KS | 183 | 16 | 118 |
| P01116-G12V | 8 | VG~G~SA | 184 | 19 | 280 |

### Example 4: Cathepsin Cleavage Profiles of Other High Frequency Tumor Driver Mutations and Passenger Mutations

Neoepitope cleavage is only one means of evasion of immune surveillance. Once a tumor mutation is expressed as a protein other modes of evasion may include failure of a mutated peptide to bind to a MHC, binding in a register that conceals the mutant amino acid within a pocket position, absence of a cognate T cell clonal population due to the T cell exposed motif being a rare amino acid motif that lacks a T cell precursor population, or any combination of these.

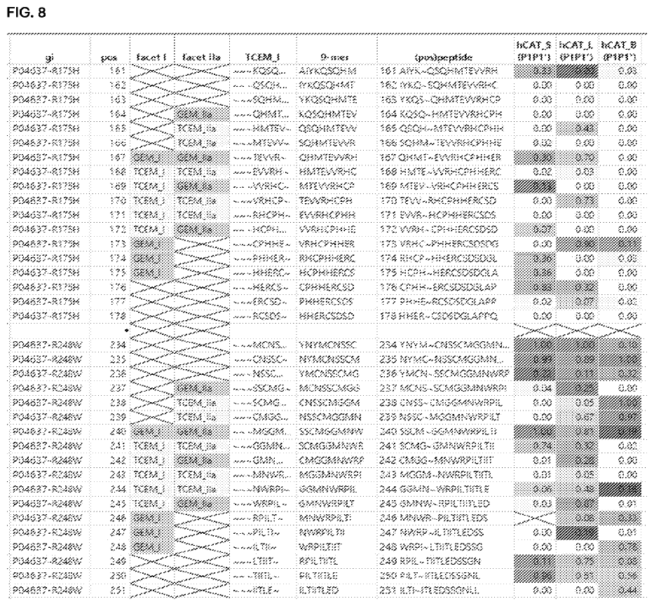

FIG. 7 shows cathepsin cleavage probability profiles for TP53, showing wild type and the most common TP53 mutant R175H. FIG. 8 shows cleavage by each position in more detail for R175H. These are very low compared to KRAS. Clearly cathepsin cleavage does not play a significant role in immune evasion of this tumor suppressor gene product.

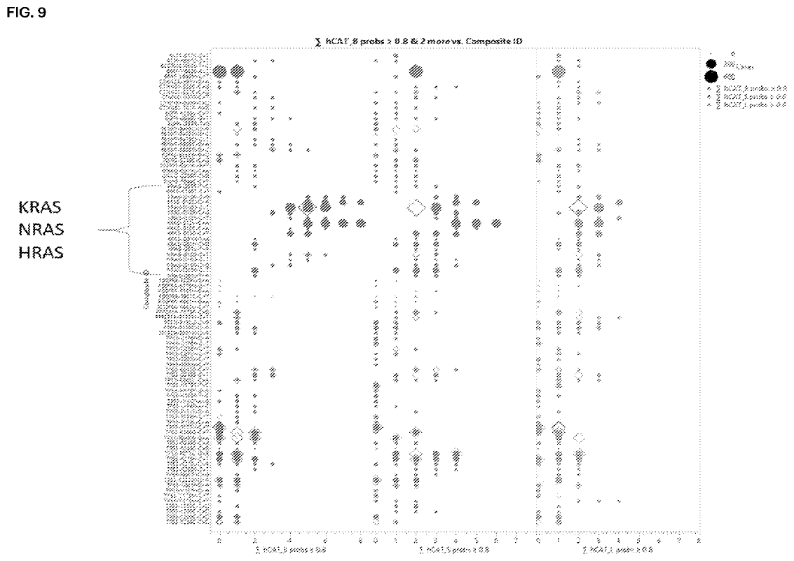

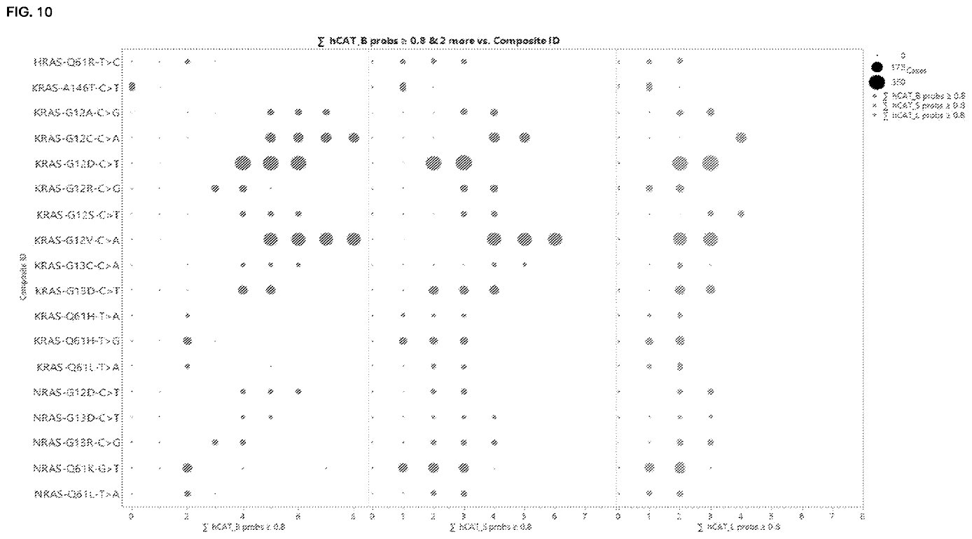

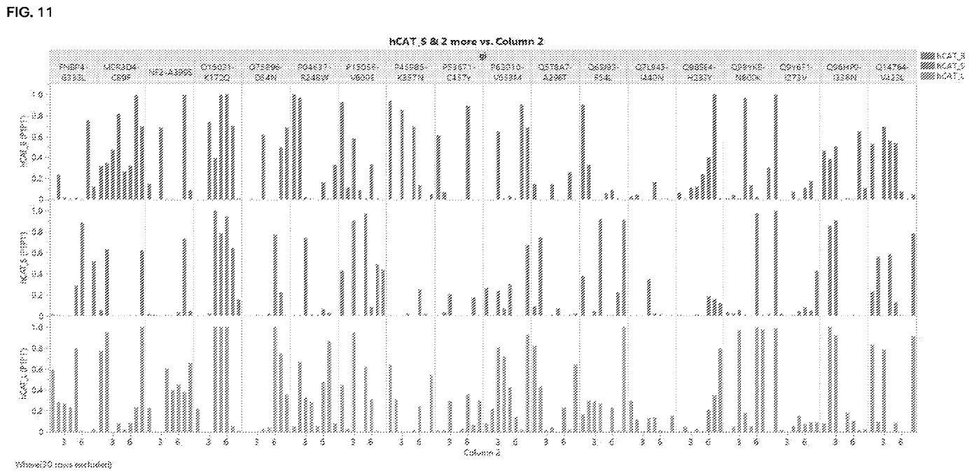

We examined the hundred most frequent tumor mutations recorded in the Genome Data Commons (81) and determined the number of cleavage sites in each 9mer that comprised the mutant amino acid that exceeded a probability of 80% of cleavage by cathepsin B, L or S. FIGS. 9 and 10 show the ranking of these mutations; FIG. 10 expands the RAS group of mutations for closer inspection. Notably the common KRAS, NRAS and HRAS mutations have by far the highest probability of cleavage and differ markedly from the other most common oncogenes and suppressors.

FIG. 11 provides an example of the diversity of cathepsin cleavage profiles found within one tumor biopsy. In this instance the biopsy was from a metastasis of a colorectal cancer and comprised a relatively limited number of expressed mutated proteins. Whole exome sequences of normal and tumor tissue were compared to identify the mutations and only the expressed proteins were analyzed. Although this biopsy example did not comprise KRAS, it does illustrate a diversity of cathepsin probabilities and indicates how individual mutations may comprise a high frequency that may benefit from cathepsin inhibition.

### Example 5: Methods of Detection of KRAS and Related Ras Mutations

In an individual cancer affected subject where tumor biopsy and normal tissue sequencing can be compared, cathepsin profiles may be derived for all expressed mutant sequences, including both passengers and drivers.

Sequences are derived from either whole exome sequencing or whole genome sequences of the tumor biopsy and a sample of normal tissue of the affected subject, or a reference unmutated human proteome. Methods for identification of mutations are well known to the art (82) and furthermore have been described in PCT Appl. US2020/037206 and US2021/062140, each of which is incorporated herein by reference in its entirety.

The process aligns normal (typically PBMCs) and tumor biopsy sequences to the reference genome. There are a number of steps in this process, such as removal of duplicates and appropriate correction of the base quality scores in the sequence before alignment to the reference and sorting by chromosome and chromosome coordinate. As the normal sample may also contain variants in the germline that differ from the reference genome, a statistical analysis contrasting the normal: reference and tumor: reference variants is used to identify the genomic coordinates where the tumor differs from the normal. With this information the translated mutant protein product can be created wherein the mutation may change the amino acid in any protein if it changes the codon at that location, lead to an insertion or deletion of an amino acid, or, if a frame shift occurs, lead to changes in downstream amino acid sequence.

Alignment and comparison of sequences, plus confirmation of expression via RNA sequencing and examination in the Integrated Genome Browser (IGV, Broad Institute) allows confirmation of the mutations. IGV enables visual comparison of the aligned DNA of the exome sequences of the tumor and the normal blood sample with those of the aligned expressed mRNA in the same genomic region.

Testing for KRAS, NRAS and HRAS mutations is almost uniformly included in oncogene panels available from a wide number of clinic and commercial sources. For example, the Memorial Sloane Kettering IMPACT Panel includes detection of the common mutants of these genes through sequencing of a selected 341 cancer genes (76); this panel specifically sequences exons 2 and 3 of KRAS and NRAS exon 3 thereby capturing mutants at the G12 and G13 positions and Q61.

Biomarker testing for KRAS mutations is widely used for colorectal, pancreatic and lung cancer, whether by PCR or hybridization and are incorporated into national and international guidelines and recommendations from the American Society of Clinical Oncology (83). Testing for KRAS mutations is included in routine colorectal cancer screening (Cologuard® Physicians Brochure) using magnetic bead hybridization capture (84). There is increasing interest in the prognostic role of KRAS mutants detected in cell free DNA (85). Given the clear picture of cathepsin degradation as a mechanism for immune evasion of mutated KRAS (and NRAS and HRAS), a positive biomarker test for KRAS is an indicator of the potential beneficial use of cathepsin B inhibitors as an intervention in affected subjects.

### Example 6: Experimental Determination of Cathepsin Cleavage

Testing of the cathepsin cleavage of KRAS neoepitopes must be done in a way that assays neoantigen presentation and differentiates from any more general impact on tumor progression due, for example, to proteolysis of extracellular matrix. We refer here to peptides comprising any of the mutants G12A, G12C, G12D, G12R or G12V as “G12x”; the choice of which of the mutant peptides to work with is determined based on availability of reagents. For in vitro testing an appropriate choice of cells can enable G13X (HCT-116) or Q61H to be tested. Both those peptides presented by MHC I or MHC II would be cleaved; however the key question in practice is expression of MHC on tumor cells which may not carry MHC II, so the primary focus is on destruction of MHC I binding 9mers by cathepsin cleavage and restoration of that by treatment with a cathepsin inhibitor.

In a cell free environment the incubation with cathepsin B, L or S of peptides derived from KRAS G12X or G13X and comprising the region of 1-25 of the protein can determine the occurrence and rate of cleavage and positions thereof. Intact versus fragmented peptide can be detected by mass spectroscopy. Reagents are commercially available and peptides of interest may be synthesized. A further step includes the provision of peptides incubated with and without cathepsin and in the presence or absence of a cathepsin inhibitor to MHC tetramers or dendritic cells, followed by the assay of T cell stimulation responses via flow cytometry or Elispot. These are methods well known to the art. Similarly, using cancer cell lines which carry a KRAS 12X or 13X mutation and treated with a cathepsin inhibitor or a control and then exposed to PBMCs from a donor can differentiate whether the cathepsin inhibition elicits a stronger response. The G12x mutants all generate one or more 9mers that are predicted to bind A*02:01 if that peptide is not cleaved. DRB1*01:01 and DRB1*04:01 also bind one or more G12x mutant-exposing 15mer to varying degrees. Given the frequency of the corresponding TCEM in GI microbiome and likely precursor population, PBMC A*02:01 donor cells are ideally used. A number of appropriate cell lines are available from ATCC as shown in Table 8. As matching of TCEM in KRAS at the mutated site to the same TCEM in the rest of the proteome and in the gastrointestinal microbiome indicates the probability of a large precursor frequency of T cell clones which are capable of response if the neoepitope peptides are intact.

TABLE 8
KRAS mutant cell lines

| Tissue source | Cell line ID at ATCC | Mutation |
|---|---|---|
| Lung | H460 | Q61H |
| | A427 | G12D |
| Colon | HCT116 | G13D |
| | LS180 | G12D |
| Pancreas | PANC-1 | G12D |
| | CFPAC-1 | G12V |
| | MIAPaCa-2 | G12C |

Many mouse models of KRAS mutations exist for pancreatic, colorectal, lung and gastric cancer. These have been recently reviewed and are thus well known to the art (86, 87). Using mice with a KRAS tumor model, treatment with a cathepsin B inhibitor and assay of T cell responses and monitoring tumor progression/regression can be used to evaluate the response to cathepsin B abrogation by a cathepsin inhibitor. One caveat is that for the mouse to respond to a neoantigen that is “rescued” by inhibition of cleavage, the recipient mouse must be immunocompetent. In one particular mouse model, the cell line CT26 derived from a C57/BL6 mouse colorectal carcinoma (88) may be used to monitor the effect of a cathepsin B inhibitor in the immunocompetent homologous C57/BL6 mouse. As the role of the cathepsin inhibitor is to “rescue” a neoantigen and thereby allow immune targeting of the mutant peptide, the use of an immunocompetent model is critical. Having evaluated the response to a cathepsin inhibitor alone, it will be obvious to those skilled in the art that it is then possible to evaluate any incremental benefit from prior neoantigen vaccination and/or co administration of a checkpoint inhibitor or other immunomodulatory therapy.

### Example 7: Protein and Peptide Inhibitors of Cathepsins

Cystatins are natural high-affinity inhibitors of cathepsins. Type I cystatins (also known as stefins) are intracellular proteins of approximately 100 amino acids. Stefin A and B are closely associated with the inactivation of cathepsins B, L and S. In contrast type II cystatins are extracellular and cystatin C is the type I cystatin most active against the cathepsins B, L and S (89). Sequences of these three cystatins are shown in Table 9.

TABLE 9
Sequences of cystatins most active in cathepsin inactivation

| SEQ | P01040\|CYTA_HUMAN Cystatin-A |
|---|---|
| ID | MIPGGLSEAKPATPEIQEIVDKVKPQLEEKTNETYGKLEAVQYKTQVVAGT |
| NO.: | NYYIKVRAGDNKYMHLKVFKSLPGQNEDLVLTGYQVDKNKDDELTGF |
| 309 | |
| SEQ | P04080\|CYTB_HUMAN Cystatin-B |
| ID | MMCGAPSATQPATAETQHIADQVRSQLEEKENKKFPVFKAVSFKSQVVAG |
| NO.: | TNYFIKVHVGDEDFVHLRVFQSLPHENKPLTLSNYQTNKAKHDELTYF |
| 310 | |
| SEQ | P01034\|CYTC_HUMAN Cystatin-C |
| ID | MAGPLRAPLLLLAILAVALAVSPAAGSSPGKPPRLVGGPMDASVEEEGVRR |
| NO.: | ALDFAVGEYNKASNDMYHSRALQVVRARKQIVAGVNYFLDVELGRTTCT |
| 311 | KTQPNLDNCPFHDQPHLKRKAFCSFQIYAVPWQGTMTLSKSTCQDA |

Cystatins play a key role in the maturation of dendritic cells by controlling the expression of cathepsins (17) and thus their administration could have unwanted effects in dendritic cells. However, in tumor cells they may be effective in inhibiting cathepsin B and thus facilitating intact peptide presentation. Indeed cystatins have been shown to have a positive effect in limiting progression, which has been attributed to inhibition of metastasis, and anti-angiogenesis (90). Recombinant cystatin, and in preferred embodiments, cystatin C is thus a potentially a useful adjunct to therapy of tumors bearing KRAS or other highly cathepsin-cleaved mutations. The entire sequence of cystatin C, which comprises only 146 amino acids or the active domains of it in a polypeptide of residues 44-144, or sub-domains thereof may be expressed as a protein or polypeptide and administered to an affected subject, or delivered encoded in a nucleotide vector for local expression. In some preferred embodiments the cystatin may be delivered intratumorally.

Many cathepsin B inhibitors are peptides, including but not limited to epoxysuccinyl peptides, mizaridine, tokaramide, leupeptin and derivatives thereof. Hence these can be delivered in a recombinant form at a tumor site or in conjunction with a neoantigen vaccine. In addition, peptide and protein cathepsin inhibitors may be administered in conjunction with other moieties which may enhance cell uptake, either as fusions, conjugates or linked protein/peptide pairs, or in other configurations of operable association. Suitable moieties for such combination with a cathepsin inhibitor include Fc receptors, immunoglobulins, subcomponents of immunoglobulins, or fatty acid comprising moieties. Furthermore, antibodies targeting proteins upregulated and expressed on the tumor cell surface or the extracellular matrix thereof may be conjugated or fused to a cathepsin inhibitor for targeted delivery to the tumor site. Those skilled in the art will recognize that such administration can be accomplished though delivery of a peptide or polypeptide sequence or by delivery of a nucleic acid sequence that encodes such a peptide or polypeptide sequence.

### Example 8: Combination Interventions

As the role of a cathepsin inhibitor is to prevent cleavage of epitopes and enable them to be presented as intact neoantigens bound in MHC and presented to T cells, the ultimate efficacy of the intervention requires the presence of cognate active T cells. We have noted above the likelihood that exposure to other matching T cell expressed motifs in the human proteome, microbiome and exogenous environment is likely to have established a precursor quorum of T cell clones. Nevertheless, the efficacy of these can be amplified by coadministration of other immunomodulatory agents. Foremost among these are the checkpoint inhibitors, including but not limited to anti CTLA4, anti PD1, anti PDL1 or anti LAG-3, as well as other antibody derivatives acting on checkpoint receptors. Other broad immunostimulants such as IL15 and analogs thereof and other stimulatory cytokines may be administered to amplify the T cell response following abrogation of cathepsin cleavage and presentation of neoantigens.

In some preferred embodiments a neoantigen vaccine may be administered in conjunction with cathepsin inhibitor treatment to ensure tumor specific T cell clones are primed.

### REFERENCES

1. Hobbs G A, Der C J, Rossman K L. RAS isoforms and mutations in cancer at a glance. J Cell Sci. 2016; 129 (7): 1287-92.
2. Bates S E. Adenocarcinoma of the Pancreas: Past, Present, Future. Semin Oncol. 2021; 48 (1): 1.
3. Asimgil H, Ertetik U, Cevik N C, Ekizce M, Dogruoz A, Gokalp M, et al. Targeting the undruggable oncogenic KRAS: the dawn of hope. JCI Insight. 2022; 7 (1).
4. Zheng-Lin B, O'Reilly E M. Pancreatic ductal adenocarcinoma in the era of precision medicine. Semin Oncol. 2021; 48 (1): 19-33.
5. Lefranc M P, Giudicelli V, Ginestoux C, Jabado-Michaloud J, Folch G, Bellahcene F, et al. IMGT, the international ImMunoGeneTics information system. Nucleic acids research. 2009; 37 (Database issue): D1006-12.
6. Vogelstein B, Papadopoulos N, Velculescu V E, Zhou S, Diaz L A, Jr., Kinzler K W. Cancer genome landscapes. Science. 2013; 339 (6127): 1546-58.
7. Hanahan D, Weinberg R A. Hallmarks of cancer: the next generation. Cell. 2011; 144 (5): 646-74.
8. Tran E, Robbins P F, Rosenberg S A. ‘Final common pathway’ of human cancer immunotherapy: targeting random somatic mutations. Nat Immunol. 2017; 18 (3): 255-62.
9. Homan E J, Bremel, R. D. Determinants of tumor immune evasion: the role of T cell exposd motif frequency and mutant amino acid exposure. Frontiers in Immunology 2023; 14.
10. Zavasnik-Bergant T, Turk B. Cysteine cathepsins in the immune response. Tissue Antigens. 2006; 67 (5): 349-55.
11. Turk V, Stoka V, Vasiljeva O, Renko M, Sun T, Turk B, et al. Cysteine cathepsins: from structure, function and regulation to new frontiers. Biochim Biophys Acta. 2012; 1824 (1): 68-88.
12. Honey K, Rudensky A Y. Lysosomal cysteine proteases regulate antigen presentation. Nature reviews Immunology. 2003; 3 (6): 472-82.
13. Honey K, Nakagawa T, Peters C, Rudensky A. Cathepsin L regulates CD4+ T cell selection independently of its effect on invariant chain: a role in the generation of positively selecting peptide ligands. J Exp Med. 2002; 195 (10): 1349-58.
14. Hsieh C S, deRoos P, Honey K, Beers C, Rudensky A Y. A role for cathepsin L and cathepsin S in peptide generation for MHC class I I presentation. J Immunol. 2002; 168 (6): 2618-25.
15. Watts C. The endosome-lysosome pathway and information generation in the immune system. Biochim Biophys Acta. 2012; 1824 (1): 14-21.
16. Moss C X, Tree T I, Watts C. Reconstruction of a pathway of antigen processing and class I I MHC peptide capture. EMBO J. 2007; 26 (8): 2137-47.
17. Unanue E R, Turk V, Neefjes J. Variations in MHC Class I I Antigen Processing and Presentation in Health and Disease. Annu Rev Immunol. 2016; 34:265-97.
18. Chapman H A. Endosomal proteases in antigen presentation. Curr Opin Immunol. 2006; 18 (1): 78-84.
19. Evnouchidou I, van Endert P. Peptide trimming by endoplasmic reticulum aminopeptidases: Role of MHC class I binding and ERAP dimerization. Human immunology. 2019; 80 (5): 290-5.
20. Colbert J D, Cruz F M, Rock K L. Cross-presentation of exogenous antigens on MHC I molecules. Curr Opin Immunol. 2020; 64:1-8.
21. Cruz F M, Chan A, Rock K L. Pathways of MHC I cross-presentation of exogenous antigens. Seminars in immunology. 2023; 66:101729.
22. Hsing L C, Rudensky A Y. The lysosomal cysteine proteases in MHC class I I antigen presentation. Immunol Rev. 2005; 207:229-41.
23. Delamarre L, Pack M, Chang H, Mellman I, Trombetta E S. Differential lysosomal proteolysis in antigen-presenting cells determines antigen fate. Science. 2005; 307 (5715): 1630-4.
24. Cavallo-Medved D, Dosescu J, Linebaugh B E, Sameni M, Rudy D, Sloane B F. Mutant K-ras regulates cathepsin B localization on the surface of human colorectal carcinoma cells. Neoplasia. 2003; 5 (6): 507-19.
25. Poreba M, Groborz K, Vizovisek M, Maruggi M, Turk D, Turk B, et al. Fluorescent probes towards selective cathepsin B detection and visualization in cancer cells and patient samples. Chem Sci. 2019; 10 (36): 8461-77.
26. Cavallo-Medved D, Sloane B F. Cell-surface cathepsin B: understanding its functional significance. Curr Top Dev Biol. 2003; 54:313-41.
27. Gocheva V, Zeng W, Ke D, Klimstra D, Reinheckel T, Peters C, et al. Distinct roles for cysteine cathepsin genes in multistage tumorigenesis. Genes & development. 2006; 20 (5): 543-56.
28. Gopinathan A, Denicola G M, Frese K K, Cook N, Karreth F A, Mayerle J, et al. Cathepsin B promotes the progression of pancreatic ductal adenocarcinoma in mice. Gut. 2012; 61 (6): 877-84.
29 Aggarwal N, Sloane B F. Cathepsin B: multiple roles in cancer. Proteomics Clin Appl. 2014; 8 (5-6): 427-37.
30. Chan A T, Baba Y, Shima K, Nosho K, Chung D C, Hung K E, et al. Cathepsin B expression and survival in colon cancer: implications for molecular detection of neoplasia. Cancer epidemiology, biomarkers & prevention: a publication of the American Association for Cancer Research, cosponsored by the American Society of Preventive Oncology. 2010; 19 (11): 2777-85.
31. Oldak L, Milewska P, Chludzinska-Kasperuk S, Grubczak K, Reszec J, Gorodkiewicz E. Cathepsin B, D and S as Potential Biomarkers of Brain Glioma Malignancy. J Clin Med.

2022; 11 (22).
32. Vasiljeva O, Korovin M, Gajda M, Brodoefel H, Bojic L, Kruger A, et al. Reduced tumour cell proliferation and delayed development of high-grade mammary carcinomas in cathepsin B-deficient mice. Oncogene. 2008; 27 (30): 4191-9.
33. Sevenich L, Pennacchio L A, Peters C, Reinheckel T. Human cathepsin L rescues the neurodegeneration and lethality in cathepsin B/L double-deficient mice. Biological chemistry. 2006; 387 (7): 885-91.
34. Fonovic M, Turk B. Cysteine cathepsins and extracellular matrix degradation. Biochim Biophys Acta. 2014; 1840 (8): 2560-70.
35. Turk V, Stoka V, Vasiljeva O, Renko M, Sun T, Turk B, et al. Cysteine cathepsins: from structure, function and regulation to new frontiers. BiochimBiophysActa. 2012; 1824 (1): 68-88.
36. Victor B C, Anbalagan A, Mohamed M M, Sloane B F, Cavallo-Medved D. Inhibition of cathepsin B activity attenuates extracellular matrix degradation and inflammatory breast cancer invasion. Breast Cancer Res. 2011; 13 (6): R115.
37. Yoon M C, Hook V, O'Donoghue A J. Cathepsin B Dipeptidyl Carboxypeptidase and Endopeptidase Activities Demonstrated across a Broad pH Range. Biochemistry. 2022; 61 (17): 1904-14.
38. Yoon M C, Solania A, Jiang Z, Christy M P, Podvin S, Mosier C, et al. Selective Neutral pH Inhibitor of Cathepsin B Designed Based on Cleavage Preferences at Cytosolic and Lysosomal pH Conditions. ACS Chem Biol. 2021; 16 (9): 1628-43.
39. Biniossek M L, Nagler D K, Becker-Pauly C, Schilling O. Proteomic identification of protease cleavage sites characterizes prime and non-prime specificity of cysteine cathepsins B, L, and S. JProteomeRes. 2011; 10 (12): 5363-73.
40. Banay-Schwartz M, Bracco F, Dahl D, Deguzman T, Turk V, Lajtha A. The pH dependence of breakdown of various purified brain proteins by cathepsin D preparations. Neurochem Int. 1985; 7 (4): 607-14.
41. Turk B, Dolenc I, Lenarcic B, Krizaj I, Turk V, Bieth J G, et al. Acidic pH as a physiological regulator of human cathepsin L activity. Eur J Biochem. 1999; 259 (3): 926-32.
42. Bremel R D, Homan E J. Recognition of higher order patterns in proteins: immunologic kernels. PloS one. 2013; 8 (7): e70115.
43. Hoglund R A, Torsetnes S B, Lossius A, Bogen B, Homan E J, Bremel R, et al. Human

Cysteine Cathepsins Degrade Immunoglobulin G In Vitro in a Predictable Manner. Int J Mol Sci. 2019; 20 (19).
44. Tholen S, Biniossek M L, Gessler A L, Muller S, Weisser J, Kizhakkedathu J N, et al. Contribution of cathepsin L to secretome composition and cleavage pattern of mouse embryonic fibroblasts. BiolChem. 2011; 392 (11): 961-71.
45. Carbone D P, Ciernik I F, Kelley M J, Smith M C, Nadaf S, Kavanaugh D, et al. Immunization with mutant p53- and K-ras-derived peptides in cancer patients: immune response and clinical outcome. Journal of clinical oncology: official journal of the American Society of Clinical Oncology. 2005; 23 (22): 5099-107.
46. Khleif S N, Abrams S I, Hamilton J M, Bergmann-Leitner E, Chen A, Bastian A, et al. A phase I vaccine trial with peptides reflecting ras oncogene mutations of solid tumors. J Immunother. 1999; 22 (2): 155-65.
47. Rahma O E, Hamilton J M, Wojtowicz M, Dakheel O, Bernstein S, Liewehr D J, et al. The immunological and clinical effects of mutated ras peptide vaccine in combination with IL-2, G M-CSF, or both in patients with solid tumors. Journal of translational medicine. 2014; 12:55.
48. Toubaji A, Achtar M, Provenzano M, Herrin V E, Behrens R, Hamilton M, et al. Pilot study of mutant ras peptide-based vaccine as an adjuvant treatment in pancreatic and colorectal cancers. Cancer Immunol Immunother. 2008; 57 (9): 1413-20.
49. Tran E, Robbins P F, Lu Y C, Prickett T D, Gartner J J, Jia L, et al. T-Cell Transfer Therapy Targeting Mutant KRAS in Cancer. The New England journal of medicine. 2016; 375 (23): 2255-62.
50. Li Y Y, Fang J, Ao G Z. Cathepsin B and L inhibitors: a patent review (2010-present). Expert Opin Ther Pat. 2017; 27 (6): 643-56.
51. Kuranaga T, Matsuda K, Sano A, Kobayashi M, Ninomiya A, Takada K, et al. Total Synthesis of the Nonribosomal Peptide Surugamide B and Identification of a New Offloading Cyclase Family. Angewandte Chemie. 2018; 57 (30): 9447-51.
52. Gornowicz A, Szymanowska A, Mojzych M, Czarnomysy R, Bielawski K, Bielawska A. The Anticancer Action of a Novel 1,2,4-Triazine Sulfonamide Derivative in Colon Cancer Cells. Molecules. 2021; 26 (7).
53. Supuran C T, Casini A, Scozzafava A. Protease inhibitors of the sulfonamide type: anticancer, antiinflammatory, and antiviral agents. Med Res Rev. 2003; 23 (5): 535-58.
54. Hook G, Jacobsen J S, Grabstein K, Kindy M, Hook V. Cathepsin B is a New Drug Target for Traumatic Brain Injury Therapeutics: Evidence for E64d as a Promising Lead Drug Candidate. Front Neurol. 2015; 6:178.
55. Fonovic M, Turk B. Cysteine cathepsins and their potential in clinical therapy and biomarker discovery. Proteomics Clin Appl. 2014; 8 (5-6): 416-26.
56. Kramer L, Turk D, Turk B. The Future of Cysteine Cathepsins in Disease Management. Trends Pharmacol Sci. 2017; 38 (10): 873-98.
57. Frlan R, Gobec S. Inhibitors of cathepsin B. Curr Med Chem. 2006; 13 (19): 2309-27.
58. Murata M, Miyashita S, Yokoo C, Tamai M, Hanada K, Hatayama K, et al. Novel epoxysuccinyl peptides. Selective inhibitors of cathepsin B, in vitro. FEBS Lett. 1991; 280 (2): 307-10.
59. Ulcakar L, Novinec M. Inhibition of Human Cathepsins B and L by Caffeic Acid and Its Derivatives. Biomolecules. 2020; 11 (1).
60. Towatari T, Nikawa T, Murata M, Yokoo C, Tamai M, Hanada K, et al. Novel epoxysuccinyl peptides. A selective inhibitor of cathepsin B, in vivo. FEBS Lett. 1991; 280 (2): 311-5.
61. Satoyoshi E. Therapeutic trials on progressive muscular dystrophy. Intern Med. 1992; 31 (7): 841-6.
62. Hook G, Hook V, Kindy M. The cysteine protease inhibitor, E64d, reduces brain amyloid-beta and improves memory deficits in Alzheimer's disease animal models by inhibiting cathepsin B, but not BACE1, beta-secretase activity. J Alzheimers Dis. 2011; 26 (2): 387-408.
63. Hook G, Hook V Y, Kindy M. Cysteine protease inhibitors reduce brain beta-amyloid and beta-secretase activity in vivo and are potential Alzheimer's disease therapeutics. Biological chemistry. 2007; 388 (9): 979-83.
64. Hook G, Reinheckel T, Ni J, Wu Z, Kindy M, Peters C, et al. Cathepsin B Gene Knockout Improves Behavioral Deficits and Reduces Pathology in Models of Neurologic Disorders. Pharmacol Rev. 2022; 74 (3): 600-29.
65. Hook V, Funkelstein L, Wegrzyn J, Bark S, Kindy M, Hook G. Cysteine Cathepsins in the secretory vesicle produce active peptides: Cathepsin L generates peptide neurotransmitters and cathepsin B produces beta-amyloid of Alzheimer's disease. Biochim Biophys Acta. 2012; 1824 (1): 89-104.
66. Hook V, Kindy M, Hook G. Cysteine protease inhibitors effectively reduce in vivo levels of brain beta-amyloid related to Alzheimer's disease. Biological chemistry. 2007; 388 (2): 247-52.
67. Ou X, Liu Y, Lei X, Li P, Mi D, Ren L, et al. Characterization of spike glycoprotein of SARS-COV-2 on virus entry and its immune cross-reactivity with SARS-COV. Nature communications. 2020; 11 (1): 1620.
68. Fusetani N, Fujita M, Nakao Y, Matsunaga S, Van Soest R W. Tokaramide A, a new cathepsin B inhibitor from the marine sponge Theonella aff. mirabilis. Bioorg Med Chem Lett. 1999; 9 (24): 3397-402.
69. Siklos M, BenAissa M, Thatcher G R. Cysteine proteases as therapeutic targets: does selectivity matter? A systematic review of calpain and cathepsin inhibitors. Acta Pharm Sin B. 2015; 5 (6): 506-19.
70. Breznik B, Mitrovic A, T T L, Kos J. Cystatins in cancer progression: More than just cathepsin inhibitors. Biochimie. 2019; 166:233-50.
71. Benjamin D, Sato, T., Cibulskis, K., Getz, G., Stewart, C., Lichtenstein, L. Calling Somatic SNVs and Indels with Mutect2. bioRXiv. 2019.
72. Ionescu A, Bilteanu L, Geicu O I, Iordache F, Stanca L, Pisoschi A M, et al. Multivariate Risk Analysis of RAS, BRAF and EGFR Mutations Allelic Frequency and Coexistence as Colorectal Cancer Predictive Biomarkers. Cancers (Basel). 2022; 14 (11).
73. Pinheiro M, Peixoto A, Rocha P, Veiga I, Pinto C, Santos C, et al. KRAS and NRAS mutational analysis in plasma ctDNA from patients with metastatic colorectal cancer by real-time PCR and digital PCR. Int J Colorectal Dis. 2022; 37 (4): 895-905.
74. van Huijgevoort NCM, Halfwerk HBG, Lekkerkerker S J, Reinten R J, Ramp F, Fockens

P, et al. Detecting KRAS mutations in pancreatic cystic neoplasms: droplet digital PCR versus targeted next-generation sequencing. HPB (Oxford). 2023; 25 (1): 155-9.
75. Buscail L, Bournet B, Cordelier P. Role of oncogenic KRAS in the diagnosis, prognosis and treatment of pancreatic cancer. Nature reviews Gastroenterology & hepatology. 2020; 17 (3): 153-68.
76. Cheng D T, Mitchell T N, Zehir A, Shah R H, Benayed R, Syed A, et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. J Mol Diagn. 2015; 17 (3): 251-64.
77. Yoon M C, Christy M P, Phan V V, Gerwick W H, Hook G, O'Donoghue A J, et al. Molecular Features of CA-074 pH-Dependent Inhibition of Cathepsin B. Biochemistry. 2022; 61 (4): 228-38.
78. UniProt C. UniProt: the universal protein knowledgebase in 2021. Nucleic acids research. 2021; 49 (D1): D480-D9.
79. Bremel R D, Homan J. Extensive T-cell epitope repertoire sharing among human proteome, gastrointestinal microbiome, and pathogenic bacteria: Implications for the definition of self. Frontiers in immunology. 2015; 6.
80. Human Microbiome Project C. A framework for human microbiome research. Nature. 2012; 486 (7402): 215-21.
81. Grossman R L, Heath A P, Ferretti V, Varmus H E, Lowy D R, Kibbe W A, et al. Toward a Shared Vision for Cancer Genomic Data. The New England journal of medicine. 2016; 375 (12): 1109-12.
82. Richters M M, Xia H, Campbell K M, Gillanders W E, Griffith O L, Griffith M. Best practices for bioinformatic characterization of neoantigens for clinical utility. Genome Med. 2019; 11 (1): 56.
83. Kerr K M, Bibeau F, Thunnissen E, Botling J, Ryska A, Wolf J, et al. The evolving landscape of biomarker testing for non-small cell lung cancer in Europe. Lung Cancer. 2021; 154:161-75.
84. Imperiale T F, Ransohoff D F, Itzkowitz S H. Multitarget stool DNA testing for colorectal-cancer screening. The New England journal of medicine. 2014; 371 (2): 187-8.
85. Bunduc S, Gede N, Vancsa S, Lillik V, Kiss S, Dembrovszky F, et al. Prognostic role of cell-free DNA biomarkers in pancreatic adenocarcinoma: A systematic review and meta-analysis. Crit Rev Oncol Hematol. 2022; 169:103548.
86. Won Y, Choi E. Mouse models of Kras activation in gastric cancer. Experimental & molecular medicine. 2022; 54 (11): 1793-8.
87. Sheridan C, Downward J. Overview of KRAS-Driven Genetically Engineered Mouse Models of Non-Small Cell Lung Cancer. Curr Protoc Pharmacol. 2015; 70:14 35 1-14 35 16.
88. Castle J C, Loewer M, Boegel S, de Graaf J, Bender C, Tadmor A D, et al. Immunomic, genomic and transcriptomic characterization of CT26 colorectal carcinoma. BMC genomics. 2014; 15 (1): 190.
89. Turk V, Stoka V, Turk D. Cystatins: biochemical and structural properties, and medical relevance. Frontiers in bioscience: a journal and virtual library. 2008; 13:5406-20.
90. Cox J L. Cystatins and cancer. Front Biosci (Landmark Ed). 2009; 14 (2): 463-74.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A method of treating a subject having a tumor comprising:
Performing or having performed an assay to identify tumor mutations on a nucleic acid sample from the subject to determine if a mutation of a protein encoded by a Ras gene is present; and
If the subject has a mutation of a protein encoded by a Ras gene, administering a cathepsin inhibitor to the subject.

2. The method of claim 1, wherein the step of performing or having performed the assay on a nucleic acid sample from the subject to determine if a mutation of a protein encoded by a Ras gene is present further comprises:
Determining the sequences of genes encoding Ras proteins in the nucleic acid sample;
Identifying amino acid mutations in the Ras proteins as compared to corresponding wild-type sequences of the Ras protein in the subject or a reference human subject; and
Identifying a mutation of a Ras protein.

3. The method of any one of claims 1 to 2, wherein the nucleic acid sample is from a source selected from the group consisting of a tumor biopsy, a tissue, a cell, collection of cells, blood, plasma, serum, organ secretion, seminal fluid, vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, and a cell-free DNA sample.

4. The method of any one of claims 1 to 3, wherein the assay to identify tumor mutations is selected from the group consisting of a hybridization assay, a nucleic acid amplification assay and a sequencing assay.

5. The method of claim 4, wherein the nucleic acid amplification assay comprises sequencing the nucleic acid after amplification.

6. The method of claim 4, wherein the assay to identify tumor mutations utilizes-an oncogene panel.

7. The method of any one of claims 1 to 6, wherein the protein encoded by a Ras gene is selected from the group consisting of KRAS, NRAS, and HRAS.

8. The method of any one of claims 1 to 7, wherein the amino acid mutation occurs at positions G12, G13 or Q61 of the Ras protein.

9. The method of claim 8, wherein the mutations at G12, G13 or Q61 result in a mutated peptide selected from the group consisting of SEQ ID NOs: 56-154.

10. The method of claim 8, wherein the mutations at G12, G13 or Q61 result in a mutated peptide selected from the group consisting of SEQ ID NOs: 210-308.

11. The method of any one of claims 1 to 10, wherein the mutation of a protein encoded by a Ras gene is in proximity to a predicted cathepsin cleavage site.

12. The method of claim 11, wherein the mutation of a protein encoded by a Ras gene is within 9 amino acids of a predicted cathepsin cleavage site with an >80% probability of cleavage.

13. The method of claim 12 wherein the cleavage site is on the N terminal side of the mutant amino acid.

14. A method of treating a subject having cancer comprising:
Performing or having performed an assay to identify tumor mutations in a nucleic acid sample from the subject to identify amino acid mutations in tumor proteins in comparison to corresponding wild-type sequences of the protein in the subject or in a reference human subject;
Identifying 9mer amino acid peptides which comprise the identified amino acid mutations in the tumor proteins;
Determining the probability of cleavage by a cathepsin of each octomer centered on a potential scissile bond within any 9mer peptide that comprises an identified amino acid mutation in the tumor proteins;
Identifying the mutated tumor proteins which have a probability of cathepsin cleavage within such octomers that exceeds a predetermined score; and
If the subject has one or more mutated tumor proteins for which the cathepsin cleavage score for peptides comprising the mutant exceeds the predetermined score, treating the subject with a cathepsin inhibitor.

15. The method of claim 14 wherein the predetermined score is a greater than 80% probability of cleavage by one or more of cathepsin B, L or S at four or more of the 8 possible cleavage sites in a 9mer that comprises a mutant amino acid.

16. The method of claim 14 wherein the predetermined score is a greater than 90% probability of cleavage of one or more of cathepsin B, L or S at four or more of the 8 possible cleavage sites in a 9mer that comprises a mutant amino acid.

17. The method of claim 14 wherein the predetermined score is a greater than 80% probability of cleavage by cathepsin B at four or more of the 8 possible cleavage sites in a 9mer that comprises a mutant amino acid.

18. The method of claim 14 wherein the predetermined score is a greater than 90% probability of cleavage by cathepsin B at four or more of the 8 possible cleavage sites in a 9mer that comprises a mutant amino acid.

19. The method of claim 14 wherein the predetermined score is a greater than 80% probability of cleavage by one or more of cathepsin B, L or S at one or more of the 8 possible cleavage sites in a 9mer that comprises a mutant amino acid.

20. The method of claim 14 wherein the predetermined score is a greater than 90% probability of cleavage of one or more of cathepsin B, L or S at one or more of the 8 possible cleavage sites in a 9mer that comprises a mutant amino acid.

21. The method of claim 14 wherein the predetermined score is a greater than 80% probability of cleavage by cathepsin B at one or more of the 8 possible cleavage sites in a 9mer that comprises a mutant amino acid.

22. The method of claim 14, wherein the predetermined score is a greater than 90% probability of cleavage by cathepsin B at one or more of the 8 possible cleavage sites in a 9mer that comprises a mutant amino acid.

23. The method of any one of claims 14 to 22, wherein the step of performing or having performed the assay to identify tumor mutations on a nucleic acid sample from the subject-comprises:
Determining the sequences of genes encoding tumor proteins in the nucleic acid sample;
Identifying amino acid mutations in the tumor proteins in comparison to corresponding wild-type sequences of the Ras protein in the subject or a reference human subject; and
Identifying a mutation in the tumor protein.

24. The method of any one of claims 14 to 23, wherein the nucleic acid sample is from a source selected from the group consisting of tumor biopsy, a tissue, a cell, collection of cells, blood, plasma, serum, organ secretion, seminal fluid, vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, and cell-free DNA sample.

25. The method of any one of claims 14 to 24, wherein the assay is selected from the group consisting of a hybridization assay, a nucleic acid amplification assay and a sequencing assay.

26. The method of claim 25, wherein the nucleic acid amplification assay comprises sequencing the nucleic acid after amplification.

27. The method of claim 25, wherein the assay to identify tumor mutations utilizes-an oncogene panel.

28. The method of any one of claims 1 to 27, wherein the cathepsin inhibitor inhibits the action of cathepsin L, cathepsin S or cathepsin B.

29. The method of claim 28, wherein the cathepsin inhibitor preferentially inhibits the action of cathepsin B.

30. The method of any one of claims 1 to 29, wherein the cathepsin inhibitor is selected from the group consisting of a nitrile derivative, a ketone derivative, an acryl hydrazine derivative, a vinyl sulfonate derivative, an epoxy succinic acid, surugamide, an aloxistatin derivative, a sulfonamide derivative and betalactam cathepsin cleavage inhibitors.

31. The method of claim 30, wherein the cathepsin inhibitor is selected from the group consisting of compounds 1 to 59 and derivatives and salts thereof.

32. The method of claim 30, wherein the cathepsin inhibitor is selected from the group consisting of compounds 1 to 59.

33. The method of any one of claims 1 to 32, wherein the cathepsin inhibitor is aloxistatin or a derivative thereof.

34. The method of any one of claims 1 to 29, wherein the cathepsin inhibitor is a naturally occurring medicinal product.

35. The method of any one of claims 1 to 34, wherein the cathepsin inhibitor is a peptide.

36. The method of claim 35, wherein the peptide is a recombinant peptide.

37. The method of claim 35, wherein the peptide is encoded in a nucleic acid for administration.

38. The method of any one of claims 1 to 29, wherein the cathepsin inhibitor is a cystatin protein or polypeptide derived therefrom.

39. The method of claim 38, wherein the cystatin protein or polypeptide derived therefrom is encoded in a nucleic acid for administration.

40. The method of any one of claims 38 to 39, wherein the cystatin protein or polypeptide is selected from the group consisting of proteins and polypeptides having SEQ ID NOs: 309-311.

41. The method of any one of claims 1 to 29, wherein the cathepsin inhibitor is an antigen binding molecule, preferably selected from the group consisting of an antibody or fragment thereof and a T cell receptor or fusion thereof.

42. The method of any one of claims 1 to 41, wherein the cathepsin inhibitor agent is operably linked to a second molecule.

43. The method of any one of claims 1 to 42, wherein the cathepsin inhibitor is administered to the subject parenterally.

44. The method of any one of claims 1 to 43, wherein the cathepsin inhibitor is administered to the subject intratumorally, orally, topically or to a mucosal surface.

45. The method of any one of claims 1 to 44, wherein the tumor is a solid tumor.

46. The method of claim 45 wherein the tumor is selected from the group consisting of a pancreatic tumor, a colorectal tumor or a lung tumor.

47. The method of any one of claims 1 to 44, wherein the tumor is a hematologic cancer.

48. The method of claim 47, wherein the hematologic cancer is an acute myeloid leukemia.

49. The method of any one of claims 1 to 48, wherein the treatment further comprises the administration of a neoantigen vaccine to the subject.

50. The method of claim 48, wherein a mutation of KRAS, NRAS or HRAS is detected at position G12, G13 or Q61 and said treatment further comprises the administration of a neoantigen vaccine that comprises any of the pentamer T cell exposed motifs in SEQ ID NOs: 1-55.

51. The method of claim 50, wherein a mutation of KRAS, NRAS or HRAS is detected at position G12, G13 or Q61 and said treatment further comprises the administration of a neoantigen vaccine that comprises any of the pentamer T cell exposed motifs in SEQ ID NOs: 155-209.

52. The method of any one of claims 50 and 51, wherein the neoantigen vaccine is a peptide that comprises the amino acids of one of said T cell exposed motifs of SEQ ID NOs: 1-55 or 155-209 and in which one or more of the amino acids not within the T cell exposed motif are substituted from those present in the tumor to change the predicted MHC binding affinity.

53. The method of claim 52, wherein the neoantigen vaccine comprises proteins and/or peptides.

54. The method of any one of claims 50 to 53, wherein the neoantigen vaccine is a nucleic acid vaccine.

55. The method of claim 54, wherein the neoantigen vaccine is an RNA vaccine.

56. The method of claims 1 to 55, further comprising administering an additional immunomodulatory intervention to the subject.

57. The method of claim 56, wherein the immunomodulatory intervention is selected from the group consisting of a checkpoint inhibitor, a cytokine and an interleukin.

58. The method of claim 57, wherein the checkpoint inhibitor is selected from the group consisting of Nivolumab, Pembrolizumab, Dostarlimab and Cemiplimab, Atezolizumab, Avelumab, Durvalumab, Ipilimumab, Tremelimumab, and Retalimab.

59. The method of claim 58, wherein the immunomodulatory intervention is a protease inhibitor.


SEQUENCE LISTING (345 sequences)

SEQ ID NO: 1 - protein, 5 aa, Synthetic construct
VVGAA

SEQ ID NO: 2 - protein, 5 aa, Synthetic construct
VGAAG

SEQ ID NO: 3 - protein, 5 aa, Synthetic construct
GAAGV

SEQ ID NO: 4 - protein, 5 aa, Synthetic construct
AAGVG

SEQ ID NO: 5 - protein, 5 aa, Synthetic construct
AGVGK

SEQ ID NO: 6 - protein, 5 aa, Synthetic construct
VVGAC

SEQ ID NO: 7 - protein, 5 aa, Synthetic construct
VGACG

SEQ ID NO: 8 - protein, 5 aa, Synthetic construct
GACGV

SEQ ID NO: 9 - protein, 5 aa, Synthetic construct
ACGVG

SEQ ID NO: 10 - protein, 5 aa, Synthetic construct
CGVGK

SEQ ID NO: 11 - protein, 5 aa, Synthetic construct
VVGAD

SEQ ID NO: 12 - protein, 5 aa, Synthetic construct
VGADG

SEQ ID NO: 13 - protein, 5 aa, Synthetic construct
GADGV

SEQ ID NO: 14 - protein, 5 aa, Synthetic construct
ADGVG

SEQ ID NO: 15 - protein, 5 aa, Synthetic construct
DGVGK

SEQ ID NO: 16 - protein, 5 aa, Synthetic construct
VVGAR

SEQ ID NO: 17 - protein, 5 aa, Synthetic construct
VGARG

SEQ ID NO: 18 - protein, 5 aa, Synthetic construct
GARGV

SEQ ID NO: 19 - protein, 5 aa, Synthetic construct
ARGVG

SEQ ID NO: 20 - protein, 5 aa, Synthetic construct
RGVGK

SEQ ID NO: 21 - protein, 5 aa, Synthetic construct
VVGAS

SEQ ID NO: 22 - protein, 5 aa, Synthetic construct
VGASG

SEQ ID NO: 23 - protein, 5 aa, Synthetic construct
GASGV

SEQ ID NO: 24 - protein, 5 aa, Synthetic construct
ASGVG

SEQ ID NO: 25 - protein, 5 aa, Synthetic construct
SGVGK

SEQ ID NO: 26 - protein, 5 aa, Synthetic construct
VVGAV

SEQ ID NO: 27 - protein, 5 aa, Synthetic construct
VGAVG

SEQ ID NO: 28 - protein, 5 aa, Synthetic construct
GAVGV

SEQ ID NO: 29 - protein, 5 aa, Synthetic construct
AVGVG

SEQ ID NO: 30 - protein, 5 aa, Synthetic construct
VGVGK

SEQ ID NO: 31 - protein, 5 aa, Synthetic construct
VGAGC

SEQ ID NO: 32 - protein, 5 aa, Synthetic construct
GAGCV

SEQ ID NO: 33 - protein, 5 aa, Synthetic construct
AGCVG

SEQ ID NO: 34 - protein, 5 aa, Synthetic construct
GCVGK

SEQ ID NO: 35 - protein, 5 aa, Synthetic construct
CVGKS

SEQ ID NO: 36 - protein, 5 aa, Synthetic construct
VGAGD

SEQ ID NO: 37 - protein, 5 aa, Synthetic construct
GAGDV

SEQ ID NO: 38 - protein, 5 aa, Synthetic construct
AGDVG

SEQ ID NO: 39 - protein, 5 aa, Synthetic construct
GDVGK

SEQ ID NO: 40 - protein, 5 aa, Synthetic construct
DVGKS

SEQ ID NO: 41 - protein, 5 aa, Synthetic construct
VGAGR

SEQ ID NO: 42 - protein, 5 aa, Synthetic construct
GAGRV

SEQ ID NO: 43 - protein, 5 aa, Synthetic construct
AGRVG

SEQ ID NO: 44 - protein, 5 aa, Synthetic construct
GRVGK

SEQ ID NO: 45 - protein, 5 aa, Synthetic construct
RVGKS

SEQ ID NO: 46 - protein, 5 aa, Synthetic construct
VGAGV

SEQ ID NO: 47 - protein, 5 aa, Synthetic construct
GAGVV

SEQ ID NO: 48 - protein, 5 aa, Synthetic construct
AGVVG

SEQ ID NO: 49 - protein, 5 aa, Synthetic construct
GVVGK

SEQ ID NO: 50 - protein, 5 aa, Synthetic construct
VVGKS

SEQ ID NO: 51 - protein, 5 aa, Synthetic construct
DTAGH

SEQ ID NO: 52 - protein, 5 aa, Synthetic construct
TAGHE

SEQ ID NO: 53 - protein, 5 aa, Synthetic construct
AGHEE

SEQ ID NO: 54 - protein, 5 aa, Synthetic construct
GHEEY

SEQ ID NO: 55 - protein, 5 aa, Synthetic construct
HEEYS

SEQ ID NO: 56 - protein, 9 aa, Synthetic construct
YKLVVVGAA

SEQ ID NO: 57 - protein, 9 aa, Synthetic construct
KLVVVGAAG

SEQ ID NO: 58 - protein, 9 aa, Synthetic construct
LVVVGAAGV

SEQ ID NO: 59 - protein, 9 aa, Synthetic construct
VVVGAAGVG

SEQ ID NO: 60 - protein, 9 aa, Synthetic construct
VVGAAGVGK

SEQ ID NO: 61 - protein, 9 aa, Synthetic construct
VGAAGVGKS

SEQ ID NO: 62 - protein, 9 aa, Synthetic construct
GAAGVGKSA

SEQ ID NO: 63 - protein, 9 aa, Synthetic construct
AAGVGKSAL

SEQ ID NO: 64 - protein, 9 aa, Synthetic construct
AGVGKSALT

SEQ ID NO: 65 - protein, 9 aa, Synthetic construct
YKLVVVGAC

SEQ ID NO: 66 - protein, 9 aa, Synthetic construct
KLVVVGACG

SEQ ID NO: 67 - protein, 9 aa, Synthetic construct
LVVVGACGV

SEQ ID NO: 68 - protein, 9 aa, Synthetic construct
VVVGACGVG

SEQ ID NO: 69 - protein, 9 aa, Synthetic construct
VVGACGVGK

SEQ ID NO: 70 - protein, 9 aa, Synthetic construct
VGACGVGKS

SEQ ID NO: 71 - protein, 9 aa, Synthetic construct
GACGVGKSA

SEQ ID NO: 72 - protein, 9 aa, Synthetic construct
ACGVGKSAL

SEQ ID NO: 73 - protein, 9 aa, Synthetic construct
CGVGKSALT

SEQ ID NO: 74 - protein, 9 aa, Synthetic construct
YKLVVVGAD

SEQ ID NO: 75 - protein, 9 aa, Synthetic construct
KLVVVGADG

SEQ ID NO: 76 - protein, 9 aa, Synthetic construct
LVVVGADGV

SEQ ID NO: 77 - protein, 9 aa, Synthetic construct
VVVGADGVG

SEQ ID NO: 78 - protein, 9 aa, Synthetic construct
VVGADGVGK

SEQ ID NO: 79 - protein, 9 aa, Synthetic construct
VGADGVGKS

SEQ ID NO: 80 - protein, 9 aa, Synthetic construct
GADGVGKSA

SEQ ID NO: 81 - protein, 9 aa, Synthetic construct
ADGVGKSAL

SEQ ID NO: 82 - protein, 9 aa, Synthetic construct
DGVGKSALT

SEQ ID NO: 83 - protein, 9 aa, Synthetic construct
YKLVVVGAR

SEQ ID NO: 84 - protein, 9 aa, Synthetic construct
KLVVVGARG

SEQ ID NO: 85 - protein, 9 aa, Synthetic construct
LVVVGARGV

SEQ ID NO: 86 - protein, 9 aa, Synthetic construct
VVVGARGVG

SEQ ID NO: 87 - protein, 9 aa, Synthetic construct
VVGARGVGK

SEQ ID NO: 88 - protein, 9 aa, Synthetic construct
VGARGVGKS

SEQ ID NO: 89 - protein, 9 aa, Synthetic construct
GARGVGKSA

SEQ ID NO: 90 - protein, 9 aa, Synthetic construct
ARGVGKSAL

SEQ ID NO: 91 - protein, 9 aa, Synthetic construct
RGVGKSALT

SEQ ID NO: 92 - protein, 9 aa, Synthetic construct
YKLVVVGAS

SEQ ID NO: 93 - protein, 9 aa, Synthetic construct
KLVVVGASG

SEQ ID NO: 94 - protein, 9 aa, Synthetic construct
LVVVGASGV

SEQ ID NO: 95 - protein, 9 aa, Synthetic construct
VVVGASGVG

SEQ ID NO: 96 - protein, 9 aa, Synthetic construct
VVGASGVGK

SEQ ID NO: 97 - protein, 9 aa, Synthetic construct
VGASGVGKS

SEQ ID NO: 98 - protein, 9 aa, Synthetic construct
GASGVGKSA

SEQ ID NO: 99 - protein, 9 aa, Synthetic construct
ASGVGKSAL

SEQ ID NO: 100 - protein, 9 aa, Synthetic construct
SGVGKSALT

SEQ ID NO: 101 - protein, 9 aa, Synthetic construct
YKLVVVGAV

SEQ ID NO: 102 - protein, 9 aa, Synthetic construct
KLVVVGAVG

SEQ ID NO: 103 - protein, 9 aa, Synthetic construct
LVVVGAVGV

SEQ ID NO: 104 - protein, 9 aa, Synthetic construct
VVVGAVGVG

SEQ ID NO: 105 - protein, 9 aa, Synthetic construct
VVGAVGVGK

SEQ ID NO: 106 - protein, 9 aa, Synthetic construct
VGAVGVGKS

SEQ ID NO: 107 - protein, 9 aa, Synthetic construct
GAVGVGKSA

SEQ ID NO: 108 - protein, 9 aa, Synthetic construct
AVGVGKSAL

SEQ ID NO: 109 - protein, 9 aa, Synthetic construct
VGVGKSALT

SEQ ID NO: 110 - protein, 9 aa, Synthetic construct
KLVVVGAGC

SEQ ID NO: 111 - protein, 9 aa, Synthetic construct
LVVVGAGCV

SEQ ID NO: 112 - protein, 9 aa, Synthetic construct
VVVGAGCVG

SEQ ID NO: 113 - protein, 9 aa, Synthetic construct
VVGAGCVGK

SEQ ID NO: 114 - protein, 9 aa, Synthetic construct
VGAGCVGKS

SEQ ID NO: 115 - protein, 9 aa, Synthetic construct
GAGCVGKSA

SEQ ID NO: 116 - protein, 9 aa, Synthetic construct
AGCVGKSAL

SEQ ID NO: 117 - protein, 9 aa, Synthetic construct
GCVGKSALT

SEQ ID NO: 118 - protein, 9 aa, Synthetic construct
CVGKSALTI

SEQ ID NO: 119 - protein, 9 aa, Synthetic construct
KLVVVGAGD

SEQ ID NO: 120 - protein, 9 aa, Synthetic construct
LVVVGAGDV

SEQ ID NO: 121 - protein, 9 aa, Synthetic construct
VVVGAGDVG

SEQ ID NO: 122 - protein, 9 aa, Synthetic construct
VVGAGDVGK

SEQ ID NO: 123 - protein, 9 aa, Synthetic construct
VGAGDVGKS

SEQ ID NO: 124 - protein, 9 aa, Synthetic construct
GAGDVGKSA

SEQ ID NO: 125 - protein, 9 aa, Synthetic construct
AGDVGKSAL

SEQ ID NO: 126 - protein, 9 aa, Synthetic construct
GDVGKSALT

SEQ ID NO: 127 - protein, 9 aa, Synthetic construct
DVGKSALTI

SEQ ID NO: 128 - protein, 9 aa, Synthetic construct
KLVVVGAGR

SEQ ID NO: 129 - protein, 9 aa, Synthetic construct
LVVVGAGRV

SEQ ID NO: 130 - protein, 9 aa, Synthetic construct
VVVGAGRVG

SEQ ID NO: 131 - protein, 9 aa, Synthetic construct
VVGAGRVGK

SEQ ID NO: 132 - protein, 9 aa, Synthetic construct
VGAGRVGKS

SEQ ID NO: 133 - protein, 9 aa, Synthetic construct
GAGRVGKSA

SEQ ID NO: 134 - protein, 9 aa, Synthetic construct
AGRVGKSAL

SEQ ID NO: 135 - protein, 9 aa, Synthetic construct
GRVGKSALT

SEQ ID NO: 136 - protein, 9 aa, Synthetic construct
RVGKSALTI

SEQ ID NO: 137 - protein, 9 aa, Synthetic construct
KLVVVGAGV

SEQ ID NO: 138 - protein, 9 aa, Synthetic construct
LVVVGAGVV

SEQ ID NO: 139 - protein, 9 aa, Synthetic construct
VVVGAGVVG

SEQ ID NO: 140 - protein, 9 aa, Synthetic construct
VVGAGVVGK

SEQ ID NO: 141 - protein, 9 aa, Synthetic construct
VGAGVVGKS

SEQ ID NO: 142 - protein, 9 aa, Synthetic construct
GAGVVGKSA

SEQ ID NO: 143 - protein, 9 aa, Synthetic construct
AGVVGKSAL

SEQ ID NO: 144 - protein, 9 aa, Synthetic construct
GVVGKSALT

SEQ ID NO: 145 - protein, 9 aa, Synthetic construct
VVGKSALTI

SEQ ID NO: 146 - protein, 9 aa, Synthetic construct
LDILDTAGH

SEQ ID NO: 147 - protein, 9 aa, Synthetic construct
DILDTAGHE

SEQ ID NO: 148 - protein, 9 aa, Synthetic construct
ILDTAGHEE

SEQ ID NO: 149 - protein, 9 aa, Synthetic construct
LDTAGHEEY

SEQ ID NO: 150 - protein, 9 aa, Synthetic construct
DTAGHEEYS

SEQ ID NO: 151 - protein, 9 aa, Synthetic construct
TAGHEEYSA

SEQ ID NO: 152 - protein, 9 aa, Synthetic construct
AGHEEYSAM

SEQ ID NO: 153 - protein, 9 aa, Synthetic construct
GHEEYSAMR

SEQ ID NO: 154 - protein, 9 aa, Synthetic construct
HEEYSAMRD

SEQ ID NO: 155 - protein, 5 aa, Synthetic construct
LVVAA

SEQ ID NO: 156 - protein, 5 aa, Synthetic construct
VVGAG

SEQ ID NO: 157 - protein, 5 aa, Synthetic construct
VGAVG

SEQ ID NO: 158 - protein, 5 aa, Synthetic construct
AAVKS

SEQ ID NO: 159 - protein, 5 aa, Synthetic construct
AGGSA

SEQ ID NO: 160 - protein, 5 aa, Synthetic construct
LVVAC

SEQ ID NO: 161 - protein, 5 aa, Synthetic construct
VVGCG

SEQ ID NO: 162 - protein, 5 aa, Synthetic construct
VGCVG

SEQ ID NO: 163 - protein, 5 aa, Synthetic construct
ACVKS

SEQ ID NO: 164 - protein, 5 aa, Synthetic construct
CGGSA

SEQ ID NO: 165 - protein, 5 aa, Synthetic construct
LVVAD

SEQ ID NO: 166 - protein, 5 aa, Synthetic construct
VVGDG

SEQ ID NO: 167 - protein, 5 aa, Synthetic construct
VGDVG

SEQ ID NO: 168 - protein, 5 aa, Synthetic construct
ADVKS

SEQ ID NO: 169 - protein, 5 aa, Synthetic construct
DGGSA

SEQ ID NO: 170 - protein, 5 aa, Synthetic construct
LVVAR

SEQ ID NO: 171 - protein, 5 aa, Synthetic construct
VVGRG

SEQ ID NO: 172 - protein, 5 aa, Synthetic construct
VGRVG

SEQ ID NO: 173 - protein, 5 aa, Synthetic construct
ARVKS

SEQ ID NO: 174 - protein, 5 aa, Synthetic construct
RGGSA

SEQ ID NO: 175 - protein, 5 aa, Synthetic construct
LVVAS

SEQ ID NO: 176 - protein, 5 aa, Synthetic construct
VVGSG

SEQ ID NO: 177 - protein, 5 aa, Synthetic construct
VGSVG

SEQ ID NO: 178 - protein, 5 aa, Synthetic construct
ASVKS

SEQ ID NO: 179 - protein, 5 aa, Synthetic construct
SGGSA

SEQ ID NO: 180 - protein, 5 aa, Synthetic construct
LVVAV

SEQ ID NO: 181 - protein, 5 aa, Synthetic construct
VVGVG

SEQ ID NO: 182 - protein, 5 aa, Synthetic construct
VGVVG

SEQ ID NO: 183 - protein, 5 aa, Synthetic construct
AVVKS

SEQ ID NO: 184 - protein, 5 aa, Synthetic construct
VGGSA

SEQ ID NO: 185 - protein, 5 aa, Synthetic construct
VVGGC

SEQ ID NO: 186 - protein, 5 aa, Synthetic construct
VVACV

SEQ ID NO: 187 - protein, 5 aa, Synthetic construct
GACGK

SEQ ID NO: 188 - protein, 5 aa, Synthetic construct
GCGSA

SEQ ID NO: 189 - protein, 5 aa, Synthetic construct
CVKAL

SEQ ID NO: 190 - protein, 5 aa, Synthetic construct
VVGGD

SEQ ID NO: 191 - protein, 5 aa, Synthetic construct
VVADV

SEQ ID NO: 192 - protein, 5 aa, Synthetic construct
GADGK

SEQ ID NO: 193 - protein, 5 aa, Synthetic construct
GDGSA

SEQ ID NO: 194 - protein, 5 aa, Synthetic construct
DVKAL

SEQ ID NO: 195 - protein, 5 aa, Synthetic construct
VVGGR

SEQ ID NO: 196 - protein, 5 aa, Synthetic construct
VVARV

SEQ ID NO: 197 - protein, 5 aa, Synthetic construct
GARGK

SEQ ID NO: 198 - protein, 5 aa, Synthetic construct
GRGSA

SEQ ID NO: 199 - protein, 5 aa, Synthetic construct
RVKAL

SEQ ID NO: 200 - protein, 5 aa, Synthetic construct
VVGGV

SEQ ID NO: 201 - protein, 5 aa, Synthetic construct
VVAVV

SEQ ID NO: 202 - protein, 5 aa, Synthetic construct
GAVGK

SEQ ID NO: 203 - protein, 5 aa, Synthetic construct
GVGSA

SEQ ID NO: 204 - protein, 5 aa, Synthetic construct
VVKAL

SEQ ID NO: 205 - protein, 5 aa, Synthetic construct
ILTGH

SEQ ID NO: 206 - protein, 5 aa, Synthetic construct
LDAHE

SEQ ID NO: 207 - protein, 5 aa, Synthetic construct
TAHEY

SEQ ID NO: 208 - protein, 5 aa, Synthetic construct
GHESA

SEQ ID NO: 209 - protein, 5 aa, Synthetic construct
HEYAM

SEQ ID NO: 210 - protein, 15 aa, Synthetic construct
MTEYKLVVVGAAGVG

SEQ ID NO: 211 - protein, 15 aa, Synthetic construct
TEYKLVVVGAAGVGK

SEQ ID NO: 212 - protein, 15 aa, Synthetic construct
EYKLVVVGAAGVGKS

SEQ ID NO: 213 - protein, 15 aa, Synthetic construct
YKLVVVGAAGVGKSA

SEQ ID NO: 214 - protein, 15 aa, Synthetic construct
KLVVVGAAGVGKSAL

SEQ ID NO: 215 - protein, 15 aa, Synthetic construct
LVVVGAAGVGKSALT

SEQ ID NO: 216 - protein, 15 aa, Synthetic construct
VVVGAAGVGKSALTI

SEQ ID NO: 217 - protein, 15 aa, Synthetic construct
VVGAAGVGKSALTIQ

SEQ ID NO: 218 - protein, 15 aa, Synthetic construct
VGAAGVGKSALTIQL

SEQ ID NO: 219 - protein, 15 aa, Synthetic construct
MTEYKLVVVGACGVG

SEQ ID NO: 220 - protein, 15 aa, Synthetic construct
TEYKLVVVGACGVGK

SEQ ID NO: 221 - protein, 15 aa, Synthetic construct
EYKLVVVGACGVGKS

SEQ ID NO: 222 - protein, 15 aa, Synthetic construct
YKLVVVGACGVGKSA

SEQ ID NO: 223 - protein, 15 aa, Synthetic construct
KLVVVGACGVGKSAL

SEQ ID NO: 224 - protein, 15 aa, Synthetic construct
LVVVGACGVGKSALT

SEQ ID NO: 225 - protein, 15 aa, Synthetic construct
VVVGACGVGKSALTI

SEQ ID NO: 226 - protein, 15 aa, Synthetic construct
VVGACGVGKSALTIQ

SEQ ID NO: 227 - protein, 15 aa, Synthetic construct
VGACGVGKSALTIQL

SEQ ID NO: 228 - protein, 15 aa, Synthetic construct
MTEYKLVVVGADGVG

SEQ ID NO: 229 - protein, 15 aa, Synthetic construct
TEYKLVVVGADGVGK

SEQ ID NO: 230 - protein, 15 aa, Synthetic construct
EYKLVVVGADGVGKS

SEQ ID NO: 231 - protein, 15 aa, Synthetic construct
YKLVVVGADGVGKSA

SEQ ID NO: 232 - protein, 15 aa, Synthetic construct
KLVVVGADGVGKSAL

SEQ ID NO: 233 - protein, 15 aa, Synthetic construct
LVVVGADGVGKSALT

SEQ ID NO: 234 - protein, 15 aa, Synthetic construct
VVVGADGVGKSALTI

SEQ ID NO: 235 - protein, 15 aa, Synthetic construct
VVGADGVGKSALTIQ

SEQ ID NO: 236 - protein, 15 aa, Synthetic construct
VGADGVGKSALTIQL

SEQ ID NO: 237 - protein, 15 aa, Synthetic construct
MTEYKLVVVGARGVG

SEQ ID NO: 238 - protein, 15 aa, Synthetic construct
TEYKLVVVGARGVGK

SEQ ID NO: 239 - protein, 15 aa, Synthetic construct
EYKLVVVGARGVGKS

SEQ ID NO: 240 - protein, 15 aa, Synthetic construct
YKLVVVGARGVGKSA

SEQ ID NO: 241 - protein, 15 aa, Synthetic construct
KLVVVGARGVGKSAL

SEQ ID NO: 242 - protein, 15 aa, Synthetic construct
LVVVGARGVGKSALT

SEQ ID NO: 243 - protein, 15 aa, Synthetic construct
VVVGARGVGKSALTI

SEQ ID NO: 244 - protein, 15 aa, Synthetic construct
VVGARGVGKSALTIQ

SEQ ID NO: 245 - protein, 15 aa, Synthetic construct
VGARGVGKSALTIQL

SEQ ID NO: 246 - protein, 15 aa, Synthetic construct
MTEYKLVVVGASGVG

SEQ ID NO: 247 - protein, 15 aa, Synthetic construct
TEYKLVVVGASGVGK

SEQ ID NO: 248 - protein, 15 aa, Synthetic construct
EYKLVVVGASGVGKS

SEQ ID NO: 249 - protein, 15 aa, Synthetic construct
YKLVVVGASGVGKSA

SEQ ID NO: 250 - protein, 15 aa, Synthetic construct
KLVVVGASGVGKSAL

SEQ ID NO: 251 - protein, 15 aa, Synthetic construct
LVVVGASGVGKSALT

SEQ ID NO: 252 - protein, 15 aa, Synthetic construct
VVVGASGVGKSALTI

SEQ ID NO: 253 - protein, 15 aa, Synthetic construct
VVGASGVGKSALTIQ

SEQ ID NO: 254 - protein, 15 aa, Synthetic construct
VGASGVGKSALTIQL

SEQ ID NO: 255 - protein, 15 aa, Synthetic construct
MTEYKLVVVGAVGVG

SEQ ID NO: 256 - protein, 15 aa, Synthetic construct
TEYKLVVVGAVGVGK

SEQ ID NO: 257 - protein, 15 aa, Synthetic construct
EYKLVVVGAVGVGKS

SEQ ID NO: 258 - protein, 15 aa, Synthetic construct
YKLVVVGAVGVGKSA

SEQ ID NO: 259 - protein, 15 aa, Synthetic construct
KLVVVGAVGVGKSAL

SEQ ID NO: 260 - protein, 15 aa, Synthetic construct
LVVVGAVGVGKSALT

SEQ ID NO: 261 - protein, 15 aa, Synthetic construct
VVVGAVGVGKSALTI

SEQ ID NO: 262 - protein, 15 aa, Synthetic construct
VVGAVGVGKSALTIQ

SEQ ID NO: 263 - protein, 15 aa, Synthetic construct
VGAVGVGKSALTIQL

SEQ ID NO: 264 - protein, 15 aa, Synthetic construct
TEYKLVVVGAGCVGK

SEQ ID NO: 265 - protein, 15 aa, Synthetic construct
EYKLVVVGAGCVGKS

SEQ ID NO: 266 - protein, 15 aa, Synthetic construct
YKLVVVGAGCVGKSA

SEQ ID NO: 267 - protein, 15 aa, Synthetic construct
KLVVVGAGCVGKSAL

SEQ ID NO: 268 - protein, 15 aa, Synthetic construct
LVVVGAGCVGKSALT

SEQ ID NO: 269 - protein, 15 aa, Synthetic construct
VVVGAGCVGKSALTI

SEQ ID NO: 270 - protein, 15 aa, Synthetic construct
VVGAGCVGKSALTIQ

SEQ ID NO: 271 - protein, 15 aa, Synthetic construct
VGAGCVGKSALTIQL

SEQ ID NO: 272 - protein, 15 aa, Synthetic construct
GAGCVGKSALTIQLI

SEQ ID NO: 273 - protein, 15 aa, Synthetic construct
TEYKLVVVGAGDVGK

SEQ ID NO: 274 - protein, 15 aa, Synthetic construct
EYKLVVVGAGDVGKS

SEQ ID NO: 275 - protein, 15 aa, Synthetic construct
YKLVVVGAGDVGKSA

SEQ ID NO: 276 - protein, 15 aa, Synthetic construct
KLVVVGAGDVGKSAL

SEQ ID NO: 277 - protein, 15 aa, Synthetic construct
LVVVGAGDVGKSALT

SEQ ID NO: 278 - protein, 15 aa, Synthetic construct
VVVGAGDVGKSALTI

SEQ ID NO: 279 - protein, 15 aa, Synthetic construct
VVGAGDVGKSALTIQ

SEQ ID NO: 280 - protein, 15 aa, Synthetic construct
VGAGDVGKSALTIQL

SEQ ID NO: 281 - protein, 15 aa, Synthetic construct
GAGDVGKSALTIQLI

SEQ ID NO: 282 - protein, 15 aa, Synthetic construct
TEYKLVVVGAGRVGK

SEQ ID NO: 283 - protein, 15 aa, Synthetic construct
EYKLVVVGAGRVGKS

SEQ ID NO: 284 - protein, 15 aa, Synthetic construct
YKLVVVGAGRVGKSA

SEQ ID NO: 285 - protein, 15 aa, Synthetic construct
KLVVVGAGRVGKSAL

SEQ ID NO: 286 - protein, 15 aa, Synthetic construct
LVVVGAGRVGKSALT

SEQ ID NO: 287 - protein, 15 aa, Synthetic construct
VVVGAGRVGKSALTI

SEQ ID NO: 288 - protein, 15 aa, Synthetic construct
VVGAGRVGKSALTIQ

SEQ ID NO: 289 - protein, 15 aa, Synthetic construct
VGAGRVGKSALTIQL

SEQ ID NO: 290 - protein, 15 aa, Synthetic construct
GAGRVGKSALTIQLI

SEQ ID NO: 291 - protein, 15 aa, Synthetic construct
TEYKLVVVGAGVVGK

SEQ ID NO: 292 - protein, 15 aa, Synthetic construct
EYKLVVVGAGVVGKS

SEQ ID NO: 293 - protein, 15 aa, Synthetic construct
YKLVVVGAGVVGKSA

SEQ ID NO: 294 - protein, 15 aa, Synthetic construct
KLVVVGAGVVGKSAL

SEQ ID NO: 295 - protein, 15 aa, Synthetic construct
LVVVGAGVVGKSALT

SEQ ID NO: 296 - protein, 15 aa, Synthetic construct
VVVGAGVVGKSALTI

SEQ ID NO: 297 - protein, 15 aa, Synthetic construct
VVGAGVVGKSALTIQ

SEQ ID NO: 298 - protein, 15 aa, Synthetic construct
VGAGVVGKSALTIQL

SEQ ID NO: 299 - protein, 15 aa, Synthetic construct
GAGVVGKSALTIQLI

SEQ ID NO: 300 - protein, 15 aa, Synthetic construct
TCLLDILDTAGHEEY

SEQ ID NO: 301 - protein, 15 aa, Synthetic construct
CLLDILDTAGHEEYS

SEQ ID NO: 302 - protein, 15 aa, Synthetic construct
LLDILDTAGHEEYSA

SEQ ID NO: 303 - protein, 15 aa, Synthetic construct
LDILDTAGHEEYSAM

SEQ ID NO: 304 - protein, 15 aa, Synthetic construct
DILDTAGHEEYSAMR

SEQ ID NO: 305 - protein, 15 aa, Synthetic construct
ILDTAGHEEYSAMRD

SEQ ID NO: 306 - protein, 15 aa, Synthetic construct
LDTAGHEEYSAMRDQ

SEQ ID NO: 307 - protein, 15 aa, Synthetic construct
DTAGHEEYSAMRDQY

SEQ ID NO: 308 - protein, 15 aa, Synthetic construct
TAGHEEYSAMRDQYM

SEQ ID NO: 309 - protein, 98 aa, Homo sapiens
MIPGGLSEAKPATPEIQEIVDKVKPQLEEKTNETYGKLEAVQYKTQVVAGTNYYIKVRAGDNKYMHLKVFKSLPGQNEDLVLTGYQVDKNKDDELTGF

SEQ ID NO: 310 - protein, 98 aa, Homo sapiens
MMCGAPSATQPATAETQHIADQVRSQLEEKENKKFPVFKAVSFKSQVVAGTNYFIKVHVGDEDFVHLRVFQSLPHENKPLTLSNYQTNKAKHDELTYF

SEQ ID NO: 311 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 312-322 - skipped (11 entries)

SEQ ID NO: 323 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 324 - protein, 333 aa, Homo sapiens [sequence omitted]

SEQ ID NO: 325 - protein, 331 aa, Homo sapiens
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 326 - protein, 15 aa, Synthetic construct
NARSRPSFHPLSDEL

SEQ ID NO: 327 - protein, 13 aa, Synthetic construct
KRNTTWQAGHNFY

SEQ ID NO: 328 - protein, 15 aa, Synthetic construct
LGGPKPPQRVMFTED

SEQ ID NO: 329 - protein, 13 aa, Synthetic construct
IRDQGSCGSCWAF

SEQ ID NO: 330 - protein, 15 aa, Synthetic construct
DGCNGGYPAEAWNFW

SEQ ID NO: 331 - protein, 24 aa, Synthetic construct
VNGSRPPCTGEGDTPKCSKICEPG

SEQ ID NO: 332 - protein, 17 aa, Synthetic construct
EPGYSPTYKQDKHYGYN

SEQ ID NO: 333 - protein, 15 aa, Synthetic construct
YSVSNSEKDIMAEIY

SEQ ID NO: 334 - protein, 14 aa, Synthetic construct
NQEYREGKHSFTMA

SEQ ID NO: 335 - protein, 17 aa, Synthetic construct
FQNRKPRKGKVFQEPLF

SEQ ID NO: 336 - protein, 12 aa, Synthetic construct
KNQGQCGSCWAF

SEQ ID NO: 337 - protein, 19 aa, Synthetic construct
DCSGPQGNEGCNGGLMDYA

SEQ ID NO: 338 - protein, 24 aa, Synthetic construct
QDNGGLDSEESYPYEATEESCKYN

SEQ ID NO: 339 - protein, 13 aa, Synthetic construct
CSSEDMDHGVLVV

SEQ ID NO: 340 - protein, 17 aa, Synthetic construct
GFESTESDNNKYWLVKN

SEQ ID NO: 341 - protein, 20 aa, Synthetic construct
TYGKQYKEKNEEAVRRLIWE

SEQ ID NO: 342 - protein, 15 aa, Synthetic construct
YKSNPNRILPDSVDW

SEQ ID NO: 343 - protein, 19 aa, Synthetic construct
CSTEKYGNKGCNGGFMTTA

SEQ ID NO: 344 - protein, 18 aa, Synthetic construct
NKGIDSDASYPYKAMDQK

SEQ ID NO: 345 - protein, 15 aa, Synthetic construct
VANKGPVSVGVDARH